SEQUENCE LISTING (55 sequences)

SEQ ID NO: 1 - DNA, 648 bp, Schizophyllum commune
cgaccgagcgcgcgccacccagcctatcccgcgcgggtcgggacccaaaataagcgggccccgccgcgccccgtcgggcgagcgggtgtatctacgaacggaactgggaggcgactcggaagagtttggttagaaaggggaacaccatcgcggacggcccagtgctctggdcagctgagcgtgcattgtgttcaattctgacctgtggcatgtaaggaacgtgctcgggatcggagggtggcgcgagagcctcttcggtgtgagattagtaactgtactgcgaagccgcggaggggttaggatgagaggtagacagggtcgcagcccaggtgcgagaaggactgcgaaggactgttcttcgaccgcgcacctgcaattgcgcgcatggatagaatagagcgtcgccctcgagggggactcgaccagggctggtggtggcgcccgacgggactggctgggcatttgcagatggcgcgcagtccaggccgccgccgatgtgttcatcccgttttgtcagtatcgatcggatctttcgggcgtgggtataaaagcgcgccgcccgccgtctccctctttctccagcactcccatccagagcacttccctctcccatcgcatcccatcacacaataatgcccatcaccatgg

SEQ ID NO: 2 - DNA, 1033 bp, Schizophyllum commune
agcttctccggccccgaatcgaacggcaggatgtgtgggcgtgtccaatattgccatgaaaatctgtcagaagtgagccctctcgtcaccctgtacagcttcgctgagttgaaaagcagggttcatcttgggctcactgatgcactgagctcgaccggagaactaaatgaccagccggagtgttcactaacttaacgccgggtattcagggcagcttctctatgttgcgcctacgacgtagatcaccgcccatgaacgggggaaacggggaggggtgcgtttggtacgtctttacgtctggctatgttgtattgaccagcgtctgcagaagatgggcacgacgatgcgccgagccggccagtgtcgtcggatgtccactgttgaggccatccttttgctagacagacggaagagctttggaggtgcgattcctctacgaatgggaaggggcttagatggagagtgacacgtctgagctccccaacacgccttcgccgagggtgcgtctccgcggacattcacctcagttcattgttctgacctgcctaattgtatagaccggccaacaaccttgctgacgcccatcataacagtgccctgcacagagccttcccactcagtcggcgcctccctcaatcaatcccactaactcgccggctctgccccttcgccgctcgacacgtcgcttggaagagcccgggcacgggcgtccgctccccccttccctccgcgtcgtcatgcacgcagcgttaatgttgctgcaggcgagccgtaagtatattcaaaggcgtagcgaatgaatagcaggcgcgcggggacctggcacgcgcggcatgaacatgcagacttgggtgacgataacttgaactcagacgcggcgaatgaatatccaaacgcgcgggaagaaaataatttacgggagcctccccaggtataaaagcccctcacccgctcactctttctccagtcgaacaccccagttcaactacccagcccttccttccttcgctatccttcyttacaacctgctcgc

SEQ ID NO: 3 - DNA, 2527 bp, Pycnoporus cinnabarinus
agatctccgaaccagaaatgcgattgcgttcaggcccaattaagaataaagctgcgtcagggcagcgacgtatcttgatccatcattgactcaccggcatcggcgtcaacaccaaagcaagctcgtcccacccataggcgtgcaccggccggcgtgcgccattgaggtacatgagcggggcgaaagtccgccattggtagccctgtcgtggacgcgcggcgatgaaacgtttcccaccattgggaagaaacgtctgcggcccatcatcccttcaccggatgacaaggcggcgtcgcgcctttgccgcagaggccggcgggcgacatgcacagcgaaggtccgttgcggatgggaagcaggcaatcagtgggtgtcctacgccgccacgatggtcggggagcgtaggcgccctcccataaggcggcaagcatcatgatgctctccgattcgggaagcctggtgcgatgctggagagactctctccgagagaccagtgtgcgcaacgttcctggcctggaagactttaaagtgagtgtagaagggcgagcagaggacgatcatcggattgcaggaaccatcggcatcctcagcctgggaaggatggctcttggtagacattcgcggaaggtgtcctagatgtgagcgggcttcttggatgatcatgtcgtaactttttctgacctcgtcggtggtacgcatggcaggattgagcattacggtatgcctcccattcataaacgataaccccttccttcaggttggtcatctccatagagcggcacgctctcaaggcctaggctattcacacctccttcgcaacatccctattcacggtgtctgtaaggaacgacttgtcatgggatcacatgaagtgcagcatactgttcgccggtctcgcagtacagacgctagtacgggaagtcgacatccaagcgttcagtcaccacatggcaaaaaagctgcaccatactctttatggtgagttgttcgtgagtggtatacagtcattcatgagggaatgcccaccggatagggtgtggcggccgcaatattcatcgcctggcaatagtcgatgtgcgtccttgttcaatgaatatcatgggtcacatgtggagacggttaaacagcgttgactgtgaatccctggtgtgtgttgggccgaacaggtacgttgcaggaacaccaatatctcttcggcagcccagttctttgcgagcggcacaggcaggcatcgcgcaacagatcccagccatccggcctctgacattcgggatacctgaagcccttcaggtacggagcgaagaggtgggctctctgcagcgattggcggacggatagctgtatttcctctctcaccattgggaagatgtgaaaggctccatcatatagcggctcaactctacctcgaatgtccaaacacggcgggaatacttatttatgtggacaaggccgagctatgatagcttgctcccgaagttggtaagtcccgcaatctgcggttcaggcaacagtctcggaaaaataagaagaatattgtaggtgcgtgtaggcgtatcgcccaaatgcgcacacacggaggctttaggagatgaagcgcccgtgagcggtaagggagttggttcaccgccgccccgaccgactctctctctttcccagcatcatgtctcggcgcaaactttaccctctattgaccaactccacgagaaagcaggaacagcttccttgtctctcatgacgtccgcaatccagacccttagccggttcgttactcatcgttatccctgccgccatggtagtggagtcagcctggccagtgcgtagtcccgtctctcttgctgcactagagaagccccatgagacagcgttttttgctttatttctgctgtttctatagacaccataggggcaaacgatcctgcacgcccagaggtattgggctcgtcagattcccagtttttctcctcggtctgaatcggctgcacggcagataaatcggccggaaatgctatagcccttcatagcccgctatgagagtcgcaaaaggcttgtcagtcaggtcggtcgagtggctctcacgaagagcgtcaacttcgcgcgacagccgcctttcagggcaagatagatcctcccatcatcccctactgcgctcagcgccggtaccgaacaattgacttaccgacatcctccgggacgcgcaaatgctgttcgacggaacgtaatcctcttcgtcccgcctcttttcgctctcacgcattccgtgtggttcgcgcgacggccgctcatcaggaccagaccagtctcaatgtctggtaccggcacaatggtgacactgcggcaactgagtaggtctggtcactctggtgcaccgtcgcttacgctgaccttcgggatactgtcctgcagacatctggagcgcctgtctttcccctagtataaatgatgtctgtccgcaggtccttgaagaccgctcgagtcccacttgagttttaggtaggacctgtccaccaaacccctctttctgatcatg

SEQ ID NO: 4 - DNA, 2629 bp, Pycnoporus cinnabarinus
aagcgggtctctcccgcctctgttggctctcacgcattccgcatggttcgcgcgacggccgctcctcagggccagaccagtgtcaatgtctggtgccggtgcagtggtgacagtgcgacaactgagtaaggctcgtcggtttcctgccccgccgcttatgctgaccacgggcgtgttgctgtacagacttcaagggcgtctgtacttgcgtagtataaaggatgtcgctcgagagccgttcgaagacaactcgagtcccacttgagtttcgacgaggacatccctttcagcgacccttctttcagtcatgtccagattccaatctctcctctcctttgtcctcgtctctcttgccgctgtggccaacgctgccatcggtcctgtggcagacttgaccctcaccaacgccgcggtcagccccgatggcttcagccgcgaggctgttgtggtcaatgggatcacccccgctcctctcattgctggacaaaaagtgagtacatattcctagcttacgaggggtattcacgtctgaccgaactggctagggcgaccgtttccagctgaatgtcatcgacaatctgacgaaccataccatgctgaagactactagtatcgtaagatatatacactctctgttgacgccctctcgctcatacgcgctcgctagcactggcatggcttcttccagcacggtacgaattgggccgacggtgtgtcgttcgtcaaccagtgccccatcgcttcgggccattcgttcttgtacgacttccaggttcccgaccaagcaggtaatgagatttgacccgttctttcattcggcgggcctgagctcctctctctttgctagggacgttctggtatcacagccatctttccacccagtactgtgatggtttgcgggggcccttcgtcgtctacgaccccaatgatcctcaggctagcctgtatgacattgacaacggtgagcattaaatgtcccgtgaccttccctacgttctcatgcttccaccgctctcagatgacactgtcattacattggccgactggtaccacgtcgccgctaagcttggcccacgcttcccgtacgctttcttttgggcatctcggcgtttcctgtgggctcattctcggggatcataggcttggcgcggatgcaactctcatcaacgggctgggtcgaagccccggtactaccactgctgatctggcagttatcaaggtcacgcagggcaagcggttcgtatccacaatcgccctccagtcgctggctctgacacttgttcttcccgcaggtaccgattccgtttggtgtccctttcttgcgacccgaaccacacctttagcatcgatggccacactatgactgtgatcgaggcggactcagtgaatactcagcccctcgaagttgactcgattcagatcttcgccgcccagcggtactccttcgtggtaagtggaagctcgctgacatcatgccggtgatagatttctcacacattgtcatacagctggatgctagccagcccgtcgacaactactggatccgtgctaaccctgctttcggaaacgtcggattcgccggtggaatcaactctgctattctgcggtatgacggcgcacccgaggtggagcctaccacgacccaaaccacttcgacgaagcctctcaacgaggctgacttgcatcccctcacccctatgcctgtggtgcgtgtctcctaaggcccatctcactgattggatgcaaactgatggggtgcatatcagcctggtcgtcccgaggccggtggtgttgacaagcctctgaacatggtcttcaacttcgtgagcattgccttcgcgccattgtgggacaatatctgatgtccttgcagaacggcaccaacttcttcatcaacaatcactcctttgtcccaccctccgtcccggttctgctccagattctcagcggtgctcaggccgcgcaggacctcgttccggacggcagcgtctacgttctcccgagcaactcgtctattgagatctccttccccgcaactgccaatgctcctggcacccctcaccccttccacctgcacggtgtacgtccggtcctcgcgcatctgatgatgagttgatagctaacaatcacaccagcacaccttcgctgtcgtccgaagcgccggaagcagcgagtacaactacgataacccgatcttccgcgacgtcgtgagcaccggccagcccggagacaacgtcaccatccggttccagaccaacaaccccggcccctggttcctccactgccacatcgacttccacttggaagccggttttgctgtcgtcctggccgaggacactccagacacggcggcggtcaaccctgtcccccagtcgtggtcggatctgtgccccatctacgacgcacttgaccccagcgatctctgagcggtatcggtggcattatttggaaaacgttgaatacgctggacttccatcggttacggatttacggattcgggttaacattatgtacctcatacggcttggatgaaatggacggtccgtccgagcttcattgtgcttcatgttaacaaaagggttgtatgtagaataatttccagggcaatcaactcgacgctatcgatcac

SEQ ID NO: 5 - DNA, 25 bp, Artificial Sequence
  synthetic primer
ttctgaccatggcgaggttccagtc

SEQ ID NO: 6 - DNA, 30 bp, Artificial Sequence
  synthetic primer
acagtaactgattcagctcagaggtcgctg

SEQ ID NO: 7 - DNA, 34 bp, Artificial Sequence
  synthetic primer
accccctctttctgaccatggcgaggttccagtc

SEQ ID NO: 8 - DNA, 36 bp, Artificial Sequence
  synthetic primer
taacccgggcgctcagaggtcgctggggtcaagtgc

SEQ ID NO: 9 - DNA, 25 bp, Artificial Sequence
  synthetic primer
tctgatcatgtcgaggttccagtcc

SEQ ID NO: 10 - DNA, 27 bp, Artificial Sequence
  synthetic primer
gtcttcaaggacctgcggacagacatc

SEQ ID NO: 11 - DNA, 30 bp, Artificial Sequence
  synthetic primer
accaagcttagatctccgaaccagaaatgc

SEQ ID NO: 12 - DNA, 33 bp, Artificial Sequence
  synthetic primer
gactggaacctcgccatggtcagaaagaggggt

SEQ ID NO: 13 - DNA, 2000 bp, Aspergillus niger
cctcaagctgtggcccagccttcctcacccagccattttcgagctggttggactccccaagtactacgacagtttgatcgaagaggccaaccgtcggcggtgccccaagtcgaagaaggagcttagcgatcccagcatctgtctcttctgtggagatattttctgctcgcaggcagtatgctgcatggagaacaagctgggcggatgcaaccagcacgttcaaaagtaggtccattcccacctctttggggagtatctggagaactcttctaacaacgaatagatgtggcaagaatatcggtttgttcatcaacatccgcaagtgcactgtgctctacttgcacaaccacaatggatcatggcactacgcaccgtaccttgaccgacatggcgaggtcgacccgggtctccggcgcaaccgccagctgatcctcaaccagaagcgctacgaccgacttcttcgggatgtgtggttgtcgcacagcatcccggccaccatcagtcggaagctggaggcggacatcaacaacggcgggtgggagacgatctagacattggtccctgggagttattgtgttccgtgtttcagcattgttgtgtatcctgatttggcttgatgacccgtgccaaaagctcatgatgactttgcatgcattttatcgaactgtttgttcttgaaaataatttacatatatgtatctacgatgcagagggcccttgttcttgttgatggcggttgatactatatagtactctatgcgttgtgcttggcgagattcactgatgctgtcggtaatggttgtttgacagtgtgaatcttgatctgaatcgtgatttatcctatcctgcacactgaactaggtattgatctacatgtagacctcgattcgaaaccagatcagccgtgtgactcacatggagcgggtgtctcgcaaggataactttagccctggtactagcccccatacgcactgtcattagtgaggtgctgagacaatttaagctttcgcttcagccatagcttataaacagtaagattatgagagggggaaaagcttcaaccaagactattttcacccgtctttaggttgttcataatgggctacggcttcatggctctcatgcctccatcgtataatcgtcccttgtagacagtagccagctacttactagttagcacagtggacacaagccagcgaatcggggtgaagccaattagcattggatatccacgaagtatgatcgtcaccctccatgacttcacacggttgctgcatcggttcaccgggttgaacctagccgggcaggatcggagcaagtcgctggatccctcacggatcacagagctccagctccacgaagaaaagcaggatgcgatccccgcacatcggagaattgcataccgttgaagcaatccaagcaattggcaggtgtcatctccattacgtaccgtgaccttcataccctccgtatttatacctgcccctccctcccccgcttctcctgctttcttcctctctctttctctcttccatcaactcaacttccatctacttcactacttcctgtcaagtcttccatcgactttcatcacaagtcatcctctcccatgtttgtattccctgtatagaccgtactgacctcttccctagaaaccctcgtgcaatgtaattccacctgcgtcggcccctccccggggtctcgtctcaatacttcattacacacgatggaagtcatgcaatgcctttggctggactctctcaatgatcaggtatctcaggtaaatcttgggcgtggaccggtgttcgttcctatccgttggttgtgcagcattgctagcattgctgcctgccatcggctctgggttcgttctgagattatacggttaaacttgatctggataataccagcgaaaggatcatgccttccctcgtttccccccacttgatggaatggctaacaattcccagtcccacctaccacacccttaaagcaacatg

SEQ ID NO: 14 - DNA, 2000 bp, Aspergillus niger
ctcagtcaatcaactgacggaattcatgttcaacttcacccagaagagtcggcgccagcggatcaaccagcggaaccggacagagcgtctgagtgacctcctggactggaaacgcatgggccttgagtatgtcaaggctcggcagttggcactccgtcgaggtatgtgtatttccaagccactattgattagattgctcacacgttcatgcagcctatccttcgtctttcggcacgcctgatgatgtttacgacgtaatcggtggcacggagcagaagatatcgcgaccattatccgtacccggatccccgcgagatcgttctggtatgatgactcccggggattttgcctctttgcaggaggtgaaagaaggcctgagtacggaggactatgtcgcatggcgactgccgtaagtccaccaccccacccccttgggtgaatgaatgacacaaggagcgctgatgctgataaactggctgtctagaactagcgaggaagaggagccagatgaccagtatttcccgctcaccctgcgcacgaagaagaataccgaccgtccggcgtcgccgctcgatcagctgtcggtcaatggtgacagttgatcgactggtgagatagcgcggtgcgacagtctggaattctgaatagcctgcatattcgaggtaccgttcttggtggaatgatcgtgttcgcgtttacttgctctcactaatcgcccgcgtgtgaaatactgcttttggtcacccacctgctattgttatccaaagcaagcttcattcagaaaagatggtagctgatttagcccatcgagctctgatcagcatggcatttcactttgttgttccatttttttcatcttgcattgctcgtttcctccattcttgcattgttttatcccttctttgcatatgtcatcactgttgtagccagcgaattgtatcttctgattggattctaccgcagtaggtccaggttgagatagtcgaatgcacgatgaatattgtatccctacagtaacataccatcacgagctccgtgctcccgtccatcgtatccagcccgccgacgcggtaaccgataattcgtgctgttgagagcgacgatgcacaagcagctaggctgatcatccgatccgaagcagagccaagcagatccttcctcaagagtgtctagacccgccagccaggaccgaagacatccggttgttgagactagcctggccaaccatatagagttgagtcataataaccttgtccgttgtgcttccgagcgggtctcgaagcgggggagaggatgagacaaggttcatgatgaggtggttactgctggagaaccggaaaagaacgccaggagcacaccactccggcgacaggatctccaatgaggcatctgcttcgttttcgtgggggtcctggatgtgtttctcggtgagggaagacgacaatcgcggatcctaacttagtagtgggggtttagtccagggtctcgcttgaccccgctgcttcccatttatgccacgtcttctccctcctctctcgcttgctttcctttcccttcatcttcccatcttctcactatctatcttcagttatattgtttccgaataacttacttcttcttccccaacaacttcctcgttaaccgtccggtacgactcacaatgaggccgcgcacgcaggatcagactccgggtcttttcgctatgggttcagatgggtcaaggtatcgtacaatagtataacagtcactgctcgcgcatgacaggtgttcggctcgtgcttctgttcctttccttctggtttggacaggagcgcggtcgttctgagattatactgtcaaaacttgatctagataatactagcgaaaggacatgcgtggcactgattgtcccctactatttgacctacagaagacgagagggatctcgcatccctctctgttgctgacagtttccagacctttgcaattaccctcgacctgagtgtatttgtgccaaaatg

SEQ ID NO: 15 - DNA, 2000 bp, Aspergillus niger
gaccgacctcggaaatggataggttaggaaaattgggggggaagggccacacaaaaaattagcaagaaattaggagaagagtgaaagaattttgccccggaatcctctccctgatccaggtataagtagcagacggatgtcttcttgggaagatgggaagaaaaacgggctggcaggtgagcaagaggataaaaattacacaatgggttcccgaagggattctcttggtggaaaggggtggtggccacgataacaagcaacaacactgcagggaaaaaccgcgagcaagccagccactagcggctgactgtgtgggatatgctttcgtccggttttccagaggccacgggatgatgtcgatgggagggattgggttggcttgcgccgcgccgcccacttttctcccctatccagcttgcgccgcgccgacccccccctgagatttgctccttattcccccgagagggatcaatttctctgatgtatccaaaaagctatataacccgtgcaccacccacggagacaagcgcgctgaggcattgggggaagtcgttttttttttttctaccatagcgccagaggtgtgtggactaacatttctgtgctatttgcgagctagctcagggagctacggatcgtcgcgcgtttccccggcgaaaggcgctccgtacggagccgggttgtcccaaaacggcagatacccaatacgatgagtcggtgaggcatttcccgagggagcagaccaggagcacagcaacagaacacaattggggaagcttccctccttccagccgacgcggcgcaaacctgcttgcgtctcagaagtgggaattttcttgctcagaaactccggtgctggtggagccgcatgcccgattcggcccggtttgattgcgaccgagaagctggccatcatattagctgggccttggctgcttcccattggcttccgggacaccgccaagtgggggtgtaccactctatcggacccatttggatgcagggacccacccgcacgtctggttttttcccttctttgcggtctcccgttttgctgtgaaaattttgcataccgtagctggtctctattgcttgtctcttgtcttgtcctgtcttttccggtctgttgctgggttaggtggcccggtgtcagaagcggaagggagaaggggacaatttatatgtaaggaaagaatgaagggagggacccgtagagacaagacaagaatgtttttttctctcctttttgtgacgacacgagggaaaaaaggaattgaacggaagggatcggttcatacaagtgtaaaatacacacacgactacggaataatcccatcagatgcagcaatgggttatctgaaggggaaggagatgtgtgagtgaatgagagagtaagccaatgctccatcgcggaccagcacggtcaggtgaagaccctgaaaccattggctgtaccagtagtaactcccctggttacccccatcccgagtgatcccgaagggtgtgtatgtgtgtatgtgtacacagtatgtgtaaggaagtgtggtaagtgtgtatgtgcggtggaatgcccactgctttcccgggggaaggaaaaaggatgatgagccaaaaacgaggcgccaaacacggtgtaagggaaaaagaagggaaaggataaactagggataacggatgataccaaagacagacacaaacaggaaaaacaggaacaatacaatacaaacaaacggtgccaaaacaccaaacaaaaaagtaggtagggcttttttttctggtcccaacaaagcgcactaacacccgacgggggggctgggtgggaaaagggcaaaaaaccgcgaaaatttagcgggagagtatttatgtcccggggggccttctgttgtcacttttcctccagctttttcctccagaaaagttctccttccttctttcccttcccaatcccatcattttctagagaaactcctctctcagaaccaccacaaaccatcacaatg

SEQ ID NO: 16 - DNA, 1992 bp, Aspergillus niger
ctcgaggattgcctgaacattgacattcggcgtccggccgggaccaccgcggactcgaagctgcctgtgctggtctggatctttggcggaggctttgaacttggttcaaaggcgatgtatgatggtacaacgatggtatcatcgtcgatagacaagaacatgcctatcgtgtttgtagcaatgaattatcgcgtgggaggtttcgggttcttgcccggaaaggagatcctggaggacgggtccgcgaacctagggctcctggaccaacgccttgccctgcagtgggttgccgacaacatcgaggcctttggtggagacccggacaaggtgacgatttggggagaatcagcaggagccatttccgtttttgatcagatgatcttgtacgacggaaacatcacttacaaggataagcccttgttccggggggccatcatggactccggtagtgttgttcccgcagaccccgtcgatggggtcaagggacagcaagtatatgatgcggtagtggaatctgcaggctgttcctcttctaacgacaccctagcttgtctgcgtgaactagactacaccgacttcctcaatgcggcaaactccgtgccaggcattttaagctaccattctgtggcgttatcatatgtgcctcgaccggacgggacggcgttgtcggcatcaccggacgttttgggcaaagcagggaaatatgctcgggtcccgttcatcgtgggcgaccaagaggatgaggggaccttattcgccttgtttcagtccaacattacgacgatcgacgaggtggtcgactacctggcctcatacttcttctatgacgctagccgagagcagcttgaagaactagtggccctgtacccagacaccaccacgtacgggtctccgttcaggacaggcgcggccaacaactggtatccgcaatttaagcgattggccgccattctcggcgacttggtcttcaccattacccggcgggcattcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatg

SEQ ID NO: 17 - DNA, 1990 bp, Aspergillus niger
ctcgaggacaacgcatcgtttgatacacttcccgccaatatggacgttgtccagaagcctgttcagcatcgatctgggcgtctcgttctgtaagcattctcctagttactgatgactttcctctcttatctgtattccgtgaaagaggagggccactgtcctctatatagtttatggatataaaaagtttgagcttcttgccaatatgaaacagatttccccacattaagagctgtttctctataggtttccaatcaatattagtgccgtcaaaacgtttgttcagatcagattgtccacgttcgtttacagatactctgactgtagtatcatctgatctcacacgttggttgtgacgtatttttcgacgcataacattttcagcatcctgtgttatcttcgcccagtgtgaactgggtgctacagccaagtcctgttcagtgtcctttgacacagttcggttgttcagagttaccttccactcaatagtataatgaatacaaggctttcctctatgttgcctcgtagtcctttcttcgggctcctggaagaaacccagatgattgggctgggattgatgcaagggagtataaggttcatcaagtacatgttcaggtgatgggcaaaatacggatggcgtacgatctctaccgaagtcaccaggggtgggggcatacgatggagtttgtatccacggatcaggtggctgaagctgagaggcatcgtcatcgtagtaaggactaaacgtcatcccctcaaggcagtagatgccactgagaagcctagtgttgggatcatcatatgttagcctacaccatatgggtgtcccagcaagagtgtccgtgagggaagaggtgcagctaacaaaaccagtaaaatgatcaggttcatggacaatgaactaagacaggtacagtattgtagccctacccgtcttggttaacctggtaaggtcaaaaaggatcgaaccgtggctcagtacaaacaaaaggaatgttaacagtttgcgggagatgcaaggcacatgctttgtcatgtttgacgcgtttgcagtgtagaagcttccagctaccgtagattactgatacaaactcaatacactatttctataaccttactgttcaatacagtacgatcaaaatttccggaatattaatgttacggttaccttccatatgtagactagcgcacttggcattagggttcgaaatacgatcaaagagtattggggggggtgacagcagtaatgactccaactgtaaatcggcttctaggcgcgctccatctaaatgttctggctgtggtgtacaggggcataaaattacgcactacccgaatcgatagaactactcatttttatatagaagtcagaattcatggtgttttgatcattttaaatttttatatggcgggtggtgggcaactcgcttgcgcgggcaactcgcttaccgattacgttagggctgatatttacgtaaaaatcgtcaagggatgcaagaccaaagtactaaaaccccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtattgtcctgcagaatgcaatttaaactcttctgcgaatcgcttggattccccgcccctggccgtagagcttaaagtatgtcccttgtcgatgcgatgtatcacaacatataaatactagcaagggatgccatgcttggaggatagcaaccgacaacatcacatcaagctctcccttctctgaacaataaaccccacacaccgtcaaaatg

SEQ ID NO: 18 - DNA, 1500 bp, Aspergillus niger
gtcacgttgtccataattgaataaatatgagcagtcctttgtggcgtggaaacatacttagcatgtagagacaaaccttggtgcgcggcttcaggcgggcatagttagtatgctacggtaccaccgatcttattgtaccagaaaaagtcccagccagtccaatccccattctaagccacatgcatccgttcgcatgcatctgacatatcagattcgtccatctggtgcagtatctaacagaggccagagcatcaccaacatgggtaccctcagcaataatatgcatgcattgtgccccccctatggagccgtagctttcaagcaattagacacgcgcccggccgaatgagatgaaccgttggagccatcatcccactcatcccgctccagaaaggagagaaagaaaaaaaaaaaatatgaccgagcgcgtgatgaccggtgaggactccggtgaattgatttgggtgacgggagagacccaagaggggccagaataataagaatggggaaggcgaaggtaccgcctttggggtccagccacgcgactccaacatggaggggcactggactaacattattccagcaccgggatcacgggccgaaagcggcaaggccgcgcactgcccctctttttgggtgaaagagctggcagtaacttaactgtactttctggagtgaataatactactactatgaaagaccgcgatgggccgatagtagtagttacttccattacatcatctcatccgcccggttcctcgcctccgcggcagtctacgggtaggatcgtagcaaaaacccgggggatagacccgtcgtcccgagctggagttccgtataacctaggtagaaggtatcaattgaacccgaacaactggcaaaacattctcgagatcgtaggagtgagtacccggcgtgatggagggggagcacgctcattggtccgtacggcagctgccgagggggagcaggagatccaaatatcgtgagtctcctgctttgcccggtgtatgaaaccggaaaggactgctggggaactggggagcggcgcaagccgggaatcccagctgacaattgacccatcctcatgccgtggcagagcttgaggtagcttttgccccgtctgtctccccggtgtgcgcattcgactgggcgcggcatctgtgcctcctccaggagcggaggacccagtagtaagtaggcctgacctggtcgttgcgtcagtccagaggttccctcccctaccctttttctacttcccctcccccgccgctcaacttttctttcccttttactttctctctctcttcctcttcatccatcctctcttcatcacttccctcttcccttcatccaattcatcttccaagtgagtcttcctccccatctgtccctccatctttcccatcatcatctcccctcccagctcctcccctcctctcgtctcctcacgaagcttgactaaccattaccccgccacatagacacatctaaacaatg

SEQ ID NO: 19 - DNA, 2000 bp, Aspergillus niger
ttccaacaactttaacccaatcaacatgctacaaatcccaattttgaccacacccaagacacctaaaccacacccgaacctaccgttgctggcacaacaggcgtagatgaaaggtggtatccccatgttttttagcgggtgggtcccggacgaaaaggaaagagtttgtcaggtttgcccgccccgtcgaacaaacaaaaagatcaagaattaaaaataaaaattgaaaaaaaaggaggaaaggaagaagaaaatcgaaagacagaaagaggaggaccaaggtggaaaatgaaactagtcacattcgagcagaaacagtgggtcccggctggaacaattcctgtttgggtaatgtaggtctcgtgtcttctttggctgcatacgaaaatatgatgccggcacctgaaaagtgtggaagaatatctggatcggacagatacactggtcgcgtgtatatctcttgtatctactgatatagcgtggctattcagaacatgaagagtggtgataattgaatcaattaccccttagcacgcacataagactactacctgcgatcttcggacactaggatgagggataaaaaggagtacggtctgaggtatataacctctaggaacaagcatatgctactgctgtgcccttgcctacgaaatgcagcatagccatgtagtgtactccgtagaaaaactaagcttggacaatagcgactgatatttaaccggagagagtatgatagagaccaacctatgggatacttcatattaccgacgttgcccgtgtttgcgatcgctcccttggccgcccccttctgtctctctctctcgctctctctccgtacctgagaacttccgcccggagcaagaaaaacggggaaatcgcggcatgtcagccgaaagacgccgcacgcacctgatcagtcccggtggcgcgtatgaaggcccagaaaccgtggactccgtttggatgctgcagatagataagtagtagcgtagatatttaagtaactgagttttgtgagttgtcttcaaacaatttctacacaggaggccgatgcaaatttacaatttcggtcttgcaattgtcgttgtgcacgagtaagtcaagtacacagtaggaggctgagaggcaagcgttttgccatcggatatccgggaccagatggaaagaaggggcggttccccatggatggagcgcgaaggtgtggactccaagaaaacgctttggttcggtcaagacggggaaatgggagaggaatggatatcttacgaagccgaattacggagaaagggcccaaacatgtaaattatggcctgtaggtcaatcagcaaagggccgagcggtgattgcgagtacaccgatcggtagactacggaagggatgaaaaagacaggaaaatagcgagagctacatgccgtttcagaggctatcgaaatgatattccaaagtatcaccagtagccataaccactataataaaggacgaagatgagagtgccttcgttctctttgaccagaaattcactcatagtatcaaagggtatttcccaataatgtcagcggtcggagttggttactggcgcgatcgggagatatcggctcttcgttgcctgggccagcattagcgccgggtccaggttttttttttgcaaatttttttttcttttcctggctatgttttttttcgttccccctaacaatgggaaggacctccctactccgtaccgggccaaccaatccggccaatggaaactgggccgggacgcccatcgccgccgctgccactgcaaattcaggccagcgaaaaacccaagagcgtcctagcgtctccgctcgcttcttcccgcttacaagagcccttcgctcgctattttttcttccctcccttcccttctctcttcttttctttccatcccctttgaagtgtcctgtttgactggcactatcatccatctcctctctttctttccttagttttcgttcatcacagccgtcaaaatg

SEQ ID NO: 20 - DNA, 2000 bp, Aspergillus niger
ttttccagctgcagattcggagtgcagacgagccgatgaccacattcctaaaggtgggtgcgcaccagcggcttatgcggttctcatgcatctggcctgacatgctttgctgattgcggttgacagtgtaccacgtgtggtgcccgatggagagagaactgaagctactccattattagagttttggaaatccctggagaacaatgtgtgttgttcaaataatagaggggcgggggataacgtagaggatgaacgcgaagggtagaaggggacacgaaaaaggaaggctggtgctacggtgtatgattaggacgatgatctgggccccctgcgattgtgattgttcccatttgcgatgatttgcatctaaattactagagatacccctcaatggatgatgacaccaatccatagcactactatacaagagtggttaaactacacgcctactccctattgagggagctaagaaaagtatgtcagattaagcgcaaaagaagaaattttcttcccgcctcgttgatccaatttatgatttttttttttttttttttttagtacttaattattttttgattaatttttttcggttcctatcccccccctcctacaaaatttgccgccgcgaacatactctacgtactaactattcatggtaacctggcaacggcaaataatggcccgattgggattggccagatccaggtaagctaccaagttagtaggagtatagtactttgtaggaaggatcctttgtgtgtgttgtttctggtacggtaccaaatcttgagtcatttttttttctcctctcttctcttatcctctaataaccactgtcggggtcccaaagaggaaggggttggtcaagggtgggaaaagaaaaaagacaaaaatgagagaaaagagaaaggctagatacctaaccgtactgtcaggtcaagacactgagtgaggggcagtgttgatggcaaaaaaagacgtgggcaagaaaaaaagattttccctcacatgttttgccgcaccagccatcccactatcaaaaagcgatgatgtttgagattgtcgggtgtccacatcttttagtgtgaatcgctagtagaatttgggatattattgagcatcatcccatgatagcgagtacaagccccgagtaaataccaacattgctatgctgctgtgctgctatctagtttgctacgttggtcgttgacctcacagggatttccaccaaaaagtggaccgggcgggcgccactcggccgtgccacagcagcctgagagcggacaaataacaacagccgcctgccgcggggttcggttgcaaacatgaccaacaggccaggccatcatcaacccaccgctgcgttgatgcccaggatttcagtccaataatccacaatttaccaacggatagagctaggtgaattagatagacaggagggccagagggaggggaccgagatgaaaaattttcgatgaaagagtggtcaaggtggggtcgtagttcggcgctccgagggcgaggaaccaaggaaaggcgaggaaaggacaggctgatcgcgctgcgttgctgggctgcaagcgtgtccagttgagtctggaaaaggctccgccgtgaagattctgcgttggtcccgcacctgcgcggtgggggcattacccctccatgtccaatgatttcaagtcaaagccaagggttgaagcccgcccgcttagtcgccttctcgcttgacccctccatataagtatttcccctcctccccctcccacaaatttttcctttccctttcctccctcgtccgcttcagtacgtatatcttccccccctctctcttccttctcactcttctctccttctttcttgattcatcctctctctaactgacttctttgctcagcacctctacgcgttctggccgtagtatctgagcaatttttctacagactttttctatctaattccaaaaaagaacttcgagttcattcacaaccgtcatcatg

SEQ ID NO: 21 - DNA, 1500 bp, Aspergillus niger
accgcgaatacaagccggacgtgcagctcaagtacgtggatgagcatggtcgcgcgatgaaccagaaggaagcgttcaagcatctcagtcatcaattccacggcaagggcagtggcaagatgaagacggagaagcggttgaagaagatcgaagaggagaagaagcgcgaggcgatgagtgcactggacagcagtcaacataccggtatgaacaacgccatgggagcgacagcgcgcaagaaccgccaggccggtgtgcggctgggctgagtgatgttgcttctctcggactgttgtagtagatgatgaccttcaccgaagacctccatttttggaggcacaataatgatcctcagcggttactaggctatgcgatgtttgtacagattataccaatagaacgtcacatattattctatctatgtccaatagatcgatccgttttcggtggtcgataaagcaagcaagcagcacgcggttttgtccgatgttggatgggtagagaggtaagatggactcggtgctccgaagatactgcaggatgtcctcaatactccgtcgatggtgtagcagagctattgaatcgggataggcttggcgtgtgggggaatttaaccgaggccgagtgatattgtccgaggcggtggagtgcaaccacggttctgtgcatagctagctattcaaggattgttgtaaagctaccgaggatatcaagaacgtagttagcagaaggggaatggagtcaatgctgcagatttatatacaagtagtagttgatggtgagatgatgggaagtggtgactagcaagtggtaggggggtgtagattaattaccccaactcctcgtgaggggaaggccaacctcagcccatccatggattttccctcgatactaaaagagttcaccggggagagcgggacgggctcatcatttgtggtgcgatctgtcaatgagggaatccacgctccgtgatgatgacatttgacatctcatgttaatcagatagtagtcaatcagttaggacttagtagagatatatacaattctattcaagatgccattgaataaataatatactacgatggagttgcatccaagggataatatgtgcagcctgcttcttgcttcctgcttcctgcttcctgcagctgccagccatgccatgcaaaccagccaaacaagcaaaaagtcacctgctggcaatgcggagggcgtggccaatccgatgcctcccgcgtttctccccggaaactccctacaggactaactcgactagtccaaaggcagttccagtgactcaaaaaataaaataactatcgccgacctcgtctctcccgctcgtctcctcccccgattccagccttcattcaagtacttcttgccagctcccttggccccggccttttcttctgatcatctcctccctggtctcttggagtgcttgctcatttcctcctcttctttcttctcttccctgttttccatcctccattcaccatg

SEQ ID NO: 22 - DNA, 1500 bp, Aspergillus niger
tatctgcaagtaatacgaatgcatgatgatgatgtctatgtccatgggtgatagacatgcactgaaccttccatcaacatcgacggtcgatcccttccctgccgggccaggtgaccaactctaaccagaatagactttgggaccagaccatcagcgtcgcaacctcccagcaatcaaaccatttctcgcttgcaattgctactcgactaggagcgggcatatctcggggattcatccctacagggccaatcacacgagcattaaaacactaaattaagcatcctggtggcgttcagctcaaacgaattcgctctcacaacccatgtaattgggatgaaaaagaacgcccagactctcttttgttgactgcccaagcctaaagcggctccggtgagaaaaaaggagtgaccctgaattcaattgttgactggggaggatggcaatccatggttgccacccacgagcaaagcgtggctgagcccgctgaatggccggagctttgctggaattttcggttcgggtacccagcaaccccggactcacaggaccaacacactgacaccagggacggaatcacctcagggtctgctcaatcggtgtgtcaaggaaccgtgtatatttcctattgtgagctaggagatttagatccccagccggaaatagagacgggcaagacaagtgggagatctggcccgccggccacgcgagtctggctggccgttcccggcctcttttcccactcttctgtctgcaccagcccgcgtctggcctcatctccgcgcctcttctctctagatctctctggatctctctggatccaaaacagcccgcttccaaatcactggccgcgatagctcgcgtgtgttatcccgattctcatctgaccgaccatgatcgtggctgagaccggtctggggacccgtggatgatcgggatgatctcggccaaatgcctcaatttgacttttgtccgcgccgtcatagtcaatcctcggcacccccatgctctctctctctcagccagtctagtcgtctacagcttcatgttggcttggcttgtctcgaactcgggcactgagtggttccttcgtgtttatcccccactcatggcgccctcggcccaacgttcttctccagactcggtacgtacagtcgcagtgagactgcggagtctggggccccgttctcccacctcgcccgattccgtgtccagtggcgtgcagagaatgactgattccggccggtctggttgtcggtctgccgggtctggggacgaatctacctgctcgctgccatccccgcgtcccgctccaaatactacataaaggcctccatgtcccgggtctggggtagaatggatctctcatcatcttaccttctccggctccattgactctcacgcactgctacattgctcacctaatctacctaatctttgcctgactccacatcacctacctttagtcgattgctgtgcctctggtatcccaatcataccatg

SEQ ID NO: 23 - DNA, 1500 bp, Aspergillus niger
atattcatctgcgggtgaggagtcaatgagatatattgggagaattgaactatttgccggacttgcgcgatatgttgactgtcccagagagattgtaagcagattggtccctggagaggagagtatggccattttgattgggagagcaaaactgataaaattgtcggtggaaagcgccatattcaatgatcaagtgatctttcagccgagcagagttcgccatgtacgtttccgaacgggtaagctaatgtacccagtgagcatggcattccggcaatgactgtacggcggcatccaaaacacggtaagtctttctcttgttgcaacagaaattcatgaattcagacaacatccatcccccatttttataacccgtgatgccgttatacgatcaagtcaagacaaagttcccccttctaaatccaaggtcacgcacaagttgggcacctacgtttctcctcgtcaagatctcacacaggggtgagtggaccttaagtcgttcttggtaaatacccctaggagctctttctgggagacttgagtgattatgtatgcgacgttatggcaacccccttttctgtagatgcttcttgcgatggccctctagctcggacatcacttactgtaatcgtgcgcaaacagtagtagatcaagtagtcaccgggaagatgggtaagatggcacgcacaattgcgccatcattcaacatcaccattggccccaacagaacaagattgtcggccgagccattttcttgcaaggtagtcggccgacagctctcgggagggaagagagtcgaaacgctagcaagcggcagatacgaacgaacagagttgggcactcagcagtaattatgtggtagtaggcaccacattgatttggatgatgatatagatagtaatcacaataactatctacagaggaagagtagaagcggaggatgaacaagaggcctggcctcgcacggttcatctcctcctatagtttaggaacagtgccggaactagaggacggtacatgagttaaggtcagtggttcgtgccttggggaatcccaatctcgccttggaattcaggttccttctctccaaaaagatcccggagggggaggggcatttcccaatgcggccggcgctaaacatcccacctggaatgagctcatgcgggtccaatcagggccgttctgccccgtgggcggtggcagattgcgtgatcgggatcgcctgcccagcggctgtcattcgttggaggggcatccaagctgtggttcgggttcaatgtggatgttgtccccctctgcctcgtagctccatccccgttccattcacgggctcctcgtctcttggttccccgacctcgggcctcatcggggtatatttagtacttccagcttcctccgttgcattcatcttcttttgcttcctactcatccttctcttctaccattctaattaaaaaagtttatacccacccctctacaactcccttcactttcacaatg

SEQ ID NO: 24 - DNA, 1500 bp, Aspergillus niger
cataattaaataaagaatagatttcaattcaagtctatttattattttagtaattcccatcttgattgattctcccatcagcttatttaatttgactacaagttatcacatgcgactggggtaattattcccaattccaagagatccaagagaagtccccgacaattactacagacgactccaagcattcatcagcagaaggattaaatctaatgggacagtgatgtgacaccgtgactggttggcaaccaagtgtatcctgtattcccccctctatgagaaacctacttagactcaaccaggctcaccggtgaactccccatggtggtcgcatgatcagaccgcccagtgaaccgtgtccgaccgggacaatcctctaaaagtatgggaatcaccgtgattgccgcccattcccccacgtcggggctcaccggggaacatgggggaaggagtgaagagaggcaacgtgtctgtgtaagcatggatatgtcctaatttcggagtaaggatctccggggaggggaaagagagatagacatttgggaaggggaaatagggtgaagatgaggttgtattggctgagtgcagaggatttgacgatacgagtaggtaaaggaaatccggacatctgataagcacctcccgtgcagcggggggagccaggccaatgggaagggagatgtcaggtgatccgccagtctgcaggatctcttggcgtgcagggcaaatgcgagtttctctccagatttcagcaggttctagattgcgacggcgtattgcttatccttagtaggactccctaatggattccgagcaagaaaagactgtttggcgtgtaccaatggctcatagtaccagcaagagaagaattttctctctcgcttcgagaaagcaatcaaaaaaaaatcctatcctaccctaccctaccctaatacttccattgccacccgattcctcccgatagtagagcgggcgactgccattggcgggcgggccagcggattcccgccgatagataacgggcagattctgtgacctcaaactatcgactaacagcccgaacttcggcgccaccgccaaacccgccccggaagccggcctcatttgccgtttggggcgtgccaggaaatgcccgcctgcagcggagactccctagtgtggtctgtgttgcctgtgtcgtctgtgtagtatactagttactagtctactactgtacagtggatggcctgagggggggactttatgtccgactccggctgttctcctccctctatccactctaccctcttccctctcttctgtctttctccccgctctcgcccctcccctcctcgaaaacataaatcggcctttccccctcgccatcttcttcttcttctccctctcctttctctttcttcttcagactacttctctttctttcatcttttctctatattcctgttttcctagataccccagttaaaaaagttctctcaatcaatcctccccttcagaatg

SEQ ID NO: 25 - DNA, 1500 bp, Aspergillus niger
aaagtcggcacctccaggctacccggaccccgcggtatgagccacctccattaacctgctacgactggcctgttcatttcggcccgttgctcgagccaaagggggtatcacctcgactggagctactagtcttttagtcccaggatggcttcctggtttagcaggtggaccctccgcccgagccatgacgcgggtcaggtatactccagacaggaggccgaagccatgtccttcaagtgcgagagaagcgtaggctgaaccatgatatgtcacggagccatcatcacagatattgcctcggtatatccggtagacgactcgaatcatttagagactctttgcgtgtacgtggtgtgggcatgccatgagttgttgggccggcctgaaggatccatcattgggaccaagggcatcatccatgcgctacggagtactttcggagaatcagcacccctgcacaaagcattgtcaatgtgttttcttatgtcaaaagctgacagagtctgaggctcgctgacgatgggattcatgctaatgacggtccgaaagagctttcacgtaacactggtgaacatcccactcgggaagccgagacttgtgacctacttagtcaaatgagatgattatcaaagccattaaatgcctcgctgtcaggggccctggtaagtgtcttcattaatcgaaacccatcttcattcgtccccgccttcagtgctcatcattttaggtttagaagcaagattgagtgccacctgctttacaaaccagcatgggtagtctgctgttgaaattcttcaccgggagcattctggggaaggtgcaaaaggcggcgcgaagtggtcgggtcgcgattgtagtctggattggagcacaagaatcgtcagagccgaagcccgaactgagggggtctcggtcatttatcgggatgagagccaatcagcgtgcgctcatcatctgatcgtctggctgccaggcccctcaggcatcaatacggtactcggcagtatccactcccgtttctccggtgcaacaaatcatcgttggagaatccccagctcccccgccaactggggtcgatgcttctccagttgtcctggtttctcccatgaactcgcttacgataagctgctgtaccagcccaccagcacaacaatatcttcaatcaggtaggtgcttgttcgttacctgccccatcctctcctcttcttcggtcattatgaactcaattcggtcgctagctttgccgattctccgcagtccataaaaatatatctgcatttgccccttacacgtcgggaattcaccggcgcaatgagccttcgggtatggtcgcacagcgtcatgtcaataggaggctgctcctagtggtgatctactagtcgcctcaacacagcaatatataaataacaagagcattccttgagcacatctgggtaatagctgttccattctcatcaaggattacgcgaccgtgcctcgagcctccttaagcgagccatg

SEQ ID NO: 26 - DNA, 1500 bp, Aspergillus niger
ataactaatggtttgaattgcttactcagtttctttctctcggacgagcagcgaaaaagaacaggcaaggcaggacaaacgacaagcgaagtattccgatccgtggatgacaagcaccaagacaggacccatgcagtgcgttcttttttttcccctctgaaggatttgcccccattgccattgccatggggggaagttctttgatccacaccagtcaaatcagtcagtgaggatggatcgaaaagactggttccgatctcaagtctggaacggtgcagggcttgtgtgcacacacggcttattactaagggcagttattaccacgcgtcgggacagggggagatgtatttccacggcctccgaaggttggtacacaactttgactttttcgggcccagagaagggcgaagttggggttttgtggagtgcagtcaatgaatgaatggatggatgggtaaaagaaaggaacggaattgggtcgctgccatgctgggtgtgctgtggttaggggggggaaagggaggattattgagtgcctatttttgggatcaatatgactgactatggtcatggctaggactatagatttggtggtgtaagatttcattgttcatattggatgggattggcatggaatgaaagaactgaaatgaggggagagagattctagacaagtatctattatgtatctagtggatggatcgaggtctagacgtagtgagtatactaagtggactagggacaaggtaactatgtagtaggtagtgatgagtgtattccagaagtactgactgcaggcgctgtcagcacccacacgcacacgcacacacactaaatgcaccacttccacaataccctaatggagtgactacgatggatggcagatgatggcagatggcagtggattccgtctcgtggcgtctccaggaacttcttctccacctgcgcccaacttccagcacaacttggaaaaggcgaaaaagaaaagaaaaagaaaaaaagaaaaatgcaattcaatggctcgccagggacactccaatccacgtcagtggccatgtggaccccgaagccaaacaaactttttacccctgcgcctctggcggcgattcgttgaaacggccaggaacagcaggaacagcagtatgagcactagccgatctcttcgccgacggttggatgccttgccgccgcaaaaagctatataaggatgcatctcgctgcaccttttgcttcctcttcaatccatcagcaaatccactgacactctcttgatcagctttgtctgacctgtcattctctctctttttttgatacatctgattttagtctgtcgatcagactagtctgccttccttttagtcctctctcttttggactttactactacacatacaacaaaccactttctcttttttcagaggaaccaacaacagtctttcttgacaacccaaaatctcatcttttttgagtcaatctcttaaaacaaatctcatcaaaatg

SEQ ID NO: 27 - DNA, 1500 bp, Aspergillus niger
cagcgcatgtctgcagcgtgcgggcaagtccgcgagcgtctctggggtcgcttgcgcccgcgcgccttcacactgcacgatgcgtatgacgaatacaggggacctcctctctacataaacggtacatagtgtctctacggttgcacgaatgggaatttctgcttgttggaaaaaatgaatcctcctggtggatggcgaggggatgcgatcggtctggtggactggcgcgcgcctcgtgaccggcagggggaaattgtctggcgtggggtcgggggattgtgattctgggacggggtcatcttgggatgttttcttttcttttcttttcttttcttttttttattcctcctattatttgctgcgatttcaaattcttcttcactgtgtatgtgtaactaggactatgggagttgttgatcaaccgatattgaatgtttcgatttggattgaagtggagtctagatcaattggtttggttcagtatttagttagtaatggacgggtgctcgctggaagcagaagaggagctcagcccagtccccttcagtccacttcagtccacttccagatccgttcagcttagcttccgtgtggccgaacagcagataaccgatacgggccgagactagtccgtcggcgcattcctggacgaagctcggtgttgcctgcttcttctcaattgaagtgcatttatcgcaatgatatagggggggaagggtatataattgatgtcaattcaatcgtccctgtcagtagagtcatctttctccagggagaacaagaaacggttgagttcaaagtgacgtgggagaggcaacgctggacgaagcagaactatgtttggccgagtgtttgccggcgtgacgtcagcatgaatgaaaagggaatcccttgaagatgcagacaccagatgagagaaaaaaatccagatgaaacccaccagcaactgagacaggacactgtcagacccattcatcgtactgtagaagagtttgcgactcagttccagcaccattgaccctcccttccccagagggaagtggtccagcagctggcaagaccttaaatacacccagctggatgcaggctgtctagactgctccgacctcagcatcggagtaatatacaccctcctgcatgcccttcctgcagcgctggtcgatcgacagccactcagcatccggctatccccagcgatcccccatagtccatgcgggatccatattgcagtcgctccttatccccgaagacgcgctgctgctcgcctcgtctggaccggggtaagtggaacgtggcgggggttccgtaccgtatttctacctcacgacgaccctcgtctgctttcttcccccagttctttttttctccctcttaatcttcctcttcctcttcttctactcttcattcttctcttcttttcccctctccactcttcttcctccctctcctgatcagcctgtccttccttcctcttctcatccctctacctccctcacaatg

SEQ ID NO: 28 - DNA, 1500 bp, Aspergillus niger
ggcctttaggatggatcgcggccgtcaagcatcaacaccactagctgactagtgatgcttcacaccttgaaccaatagtgtatcgactctatccctcaaaatcggctaatcattcgattctttgcttattccgtgcctgacgccattgctctgctgaaacaaacacaaatcagcgaataaaggcccggctctcgtcggtggtcgctgcctacagccctttctccgcccgacagtgataatggtggctttgacgccacgcaactcccaaacgtcagacggcacaaaaatatcccacccccgggaaagggctgggtagaaggaaagggaatcccttcgaaagggggatatgccaatttgccaagcagtagaaacctcccttctatgtagctcgtctacagattctgcatctttgctgcagaacggtacacttgacgagaggtcaactatggttgcggtgtggttcttagggcacagaaaatatcagcattacatgaggggaacaagtgcatggcgcccagacgaggggaatgccttcgagtctggactgcagtggggggcgcgctgtgggcgttcatcccggccagatggtgaactaacagccactaaactagtgctgccctaaaggcgcggtctggccgctgccagacggttggccgacatggcccatcacagaaccggattgtcgagggcgattctgtcgagagaaaagactgcacaaaagtacgagataaaaatgatgttgttgggtagacatacacctccattaaagtagggggaataagtcggatacatccactggaccgatcaactgcaggtatccgcaccgctgcaggaacaaccaccgcaaggttacccccggacgcttgctgtccagtcactgccaaccgccaggcacacgggctgaataatgggcgtcaatatttctctgtcccactgtccctgagcgacacacggtacccgcccgatgacgttccatgggtcggccgcggtgaggatgcaggggggtcaggaacgctccgacgcaggcaatcagagggggtcgtcgaacaatggaaaaagcaacgattagtgactagttcgactttactcatgcaagagcaaataagaaccttcctccttgtggagacctgattggtcggaaccaaattggcgcctagaaaaagcacccagccctaacttggttctgcaactgccactccccgttgttgggcgtctatataaccgccctctttcccctccctgtctcctcttcgaaactcttcttcctcacctagatcttcctctctcacctccccagcctttccttctttgcacctgtgccgtgcacggtcgagccattccttcattctttgaacatattgcctgactccgagtagtctagcatccactccttgcaagagcactttgagagaaccggtcttctcatactcaaaagttatacatacacaacacttctctccgaacaaaaccgaacaaaattcgcagaacacatacacaatg

SEQ ID NO: 29 - DNA, 1500 bp, Aspergillus niger
gtggacttcttccggtcggaaggcttggactcggcgctggttttctcgtttcgtgtatagatggtttcgggcactgaagggtgagagaaaaaaagcaattagtaaagctagaccccaaagtgccacagataaacagagttaaacttaccaaagaagatctgggcaacgaattgtgtggcgtacaggatggctgcgatgtacatggaccaccgccatccgaggctcttattgtatgcaatcgagccaccagcgacaccgcccacatacggactaataacgagcaaaatgctatttacacccatccgcgaccccttctcatgcaagaaaaacatatcagaaatagcagcgggtccgatggacaacccaacgctgccacctacattcatgagcatgcgacaagctaaacactgcgcatatgtctgggatcgagccaccccgatcgcgctgaccaaggtcatcagattgccaatgagaagtactggccgtcgacctattcggtggctcatcgggatccagaagaaggaggttattgcgtttaagatggaaggtacagatccgatgtacgtcgcggttgtctcggggacattgaattcttcagccagctccaatgcatcaggagcctaagggatctattaggagaggagataaaaagagtaggaaagttcagtagacctacaatcaacgtggccgagaatttgaccaggaatgattcgaacgcgaggaccataagagctgcaattttggtgaattggctccaattctataggaaatccctctactgtcaggacatcattgatcagtagtttctgtgaggacagcgaacgaaccaagggatcctctggatcatcactgggctgggggaccagagggatgccttgtgcggtggtcttgattctagcattggcctccgggtcccccgatgacatggacttttcttggagaacttccacagattgaagctcctttacgtccgccattttgacaccaagcaagcgctcttgctggataattggggaagaagaagacaggggggcattcttcagaaatttatcgtcacggcaagcgccgagacgctatcgcgggggagaatattgacgcggagctccagaggcaaaatccggagtcaagtcgcgagtagaagtagtagtagtaatagataagagatcttcggaagctgtcagaactgaaaagcgaacgtgcagactggacggcatgcatgcttgccgccagttttgtgcgtgggcttaatctcgagcccgcgctatctggaccggtcgttcacgctaaggccgtggctgcatatcgccagcaatttgtagctcacgccgtccgttatagtctgttatacccctgtcaagctcgaaaatagtcacccacctccgagaatacataatcaatccctccggaacacaccggctacatgcagaccgcaactcctccttctcctccattcagccctcccggaggatctcccggttgatcaatccgcaaccaaagatg

SEQ ID NO: 30 - DNA, 1500 bp, Aspergillus niger
gagctcctcgaatccggcaacgtcggcaacaaggtcagcgtacaggtcgtccgagttcagggtggaccagaactgggtgtactgggcagactcgaggagggtggagaggcgggcgagcttctggatcgactcgacgaagtcggaggtctgggcggcggcctgggcctcagcgtcggaggcctggaagggctgagtgtgggcggggagaagagcaaggcagagggagaaagcgggagaggggaagacagtcagggccttgacgaggacgttggtgacggtctccgcctggagaaggtgggggttgaactggtacctgtcagagggcgtgaaggggttagcatcgctattcaggtatgatgtacatgcgatagaataaaagttgagatcatggttttgtttcaggagaattggagtctcgacatcccccaaagtagggtgtcaggtaataggaaacgtgataatgatataagattcttcatcacataaaaccaaaatggttccccgcgtcggagctctttatgtttcggctgaaagtgttttgaactggatagaaaggcgcatgaactcacaacttcagcaaggccaggttggcgtagcagtcgaaggtgcggtcctcgcactgctgggcgacatagtcctggaagacagtggtggtctcggggttgtagcggtcgaggccattgaggatggcgtcaatgttggccgggcgggtctcgcacttgtcgaaggtgacacccatcttgacggagatacggagaatggacgagtcggggggaagaagaggggaaagcgtgggtggaggaggaggagagggagggaagcagaacgaggggataggaagagagcagcggaatggatggactcgaggtttggttggccggacttgcttttggtgggatttttcgattgtctcgctgtgcggctgcccggccgtcggagccaaccccaactcggttaatttgtccgatgcatctgcattggcattatttttattttattcctttctattctagtcagtcagtcagtcagtcatcactctttactaaacttcactcaatactactctaaattaggcactagtggctgatctggtaaatcgtatgatatactttgtaactttgagatgaaccaaattccacgtgcccccggaatcggtctcgccgggggcaaaggatctccctgtcccgtggtgggatcatgtcggtgctggtagggatagcccgtgtcgccagtgtatttggttagtagggactcgcccccgtgtaggtttaccccgtggtaggtatatcccgcggtagggattcgccgcatatataagatgacggggataacaactccaacacagcagtagcttctgcgcctgacttctatacattcaataaattgaccgcgtatttcccttagcctttgtccgaagcacgcagttctgatcgcaaccgggatcccctcaacaaccctaacgatatccccgggctatactatacaccttaaatcgcgatg

SEQ ID NO: 31 - DNA, 1500 bp, Aspergillus niger
tgatcgcgacaatgcattggccacgcttgaatttgagggtggtaagatggctgctttgtactgcacgcgcatgatggcggcgggacaggaggatattactgaacttatctgtgaaaggggttcaattcaggtgaattttcagccgcgaaagaatcatgttgagattcatgacgctgcaggcgctagacggctgcttccacagcactattatgagcggtttagggaggctttcgttactgaggctgctgagtttactgagtgttgtttggagaataaggctccaccgatcagccttcggagctcggttagggcggtcgctgttgggcaggctttgcagcgcagtttgattagtggagagaagatatttttcgagggtgagaccttaaagaactagatagaatgaaagtgacactcacgattcaagttgttttgttgaggtgtactgagccgacctgattatggataatgatgatcgaatatgacaaataggtactgagagatagttacgaggtccgtagagtcaaacgaagcagtatggctcattgaacaactagaaacaagacttatattaatacttgataatagtagaaaacaggaaccagggatatactctagagattacagaaataatcaagctataaacagaagaaggtatcctgcctttttactatattataagattatttccagaaagaggattaggacttattctattatataatttatcttgccgtgaataatcttgaaaatatattagataaaatcagaaataaactgagcatgcctatattttataataaactcagttaggatagagcaacatactccgtatgcctctataatattcagtgcatgattaaataatttatagaagtaactgttgcatgtgcaagaccacgctgactaagtggattagtggattataacgtaacccacggttaaacggcccacacggttaggcggcccagctggtaatactatacaaaagaccctttttgaacacaagatatctcaaccattataagactagaagactcaatattgtataaatagattataaattatataaataactatccaatacatctcaaaaatctatataaatatgaatagtatcttatcatattacaaaatctgtaatttatctgtaattttggatatattgatatatttttcataaaagaaattcttaaaaatctagaaaaattatatattatttattctactgtatattatctatatattaagttagaactgattatattcagtattggttgagatatgaagatttaaaaaaacatcttttatatgggataaccagatgggccgcctaaccgtgtgggccgcttaaccgtgggttacgttatgataatctctcatggaagttcctggaggtaaagaatgttttgtttccttgtggcctatctttaagatattttagtcaagtgtagcacagttctttgtattcttgactgttgtcgagcgcagtgctttcacatg

SEQ ID NO: 32 - DNA, 1500 bp, Aspergillus niger
aatctacctgacaaatatgtttaaatccttgctaaagcagaacatgtgagattttcaaattatttattatggaagatttcaaggcccaatttgacaggttttatgtactgataaacaatcacagcttgaattgacactgaactccaataaatgtgacacatagtctgaacagcagatgctggcagaaaaattatacaatgtacatatgcaatgcaggattgaatcgctttaaccaatgaacaatgcatttatgacaaatcaacgccaagataccctcgctccagtacgtaatgcaggtattaacacatccttctccccaaacccttaggaggtgaaccacatgaatctgggataagcagaattagcggaaatcgtaaagctcaagtcaagcagatacctacatctcataggccatggagggaatatctttgcaaagtgagacaccagatcgaataagggatatggttgcacccgtcagcacgaggatggccaatggacgaaacgccagaaaccaggcatatctccacacgacccctatggcggatctgtttaaaactggtcagcacgctagttgaatttagttgaccattgtgctgcagcttacatgagagtggaggtggaagacatctcgtttagtgatgggcgtttttttctctcaacgaacaaagagtccttgtggtactattgaggaaaggttttgaaacttcaaggatgagcaaatcgattcagacatgatccttgaaagtgggaaacaagccaaggctcttatgtcaaggttatatcccatttgcttgcgccattgccaaggtggcagcctgctatcgtcattcccaacgggtctcgtccgagccacagcagcgggggtaaacgttgcaccgcgccaaagacattggatctttagatagaatcctctaatcccattcaggaaagcctgtggtaaaaatgctgcctgaacccgataatcctaccaaatcctgccttccgactttccgaaagttgtccacggggtcctcgggagctggccaaccagaagagctggctgggtcaattcgcgccagatgcgaagagtgactgggcagggcagccggaagactttgctgggggtctccactattggctcctcgggaggggaaaggggcccgctccctcagccctacaaggctgccgctgcacttttgtgaaatatcagttgacccctggccttgcgcaacgtggatgtcctttttcttactgctttctctttaaatccaaagtcactcgctctctgtctaccttggcccagccgtttacggatgaaaaccgtgtcgacgatctatgacgttttgcttgttcgatcagtcaacaagcgtttatatcatagaatgggctgagtgtacgcctttcctgcccttctctggctttgtgcgttttcactgtggttagatacgcttgtgtgtgaataatttttactgtcgctctttcgctccctgaggtggctgctcagtctggattcacaagatg

SEQ ID NO: 33 - DNA, 1500 bp, Aspergillus niger
cacggctaaggctggtgtcgtcagcccggaagaggagcaactgcgcttctgggtccggggagagaagggcagcttcaagaaggtgagacaacggcctttcaccctaggatgtcggttagtaacaaaatactgatgcgtacacgctcgatagttccatctcgacgtgcaggaagagcagctcaaggctggtgtgaagccaggtgacagcggatatggccgagagccttctgagagatacggtaagtcaaggattgccgccgttggactttcgcaacaggctaacagtctctgttaggtactctcacgactattcaggacggcaagcccgtcaaggaagtcatgcccacagtagagcccccgacctggacggaatactaccgcaagctggcgcgggctcttggtggagagggcgacctccctgccagcggcgccgaggctcgtgaagtgatccggctgatcgagttggctcaggagagctcgaaacagggaaagaccctggatgtataagtctcgtgtaaagagtgtgcctatcatgggtcatgatttaatgatggatagaattgttttcgcattgtacgagtggtagtgtgaagtttttagattgtttctctttgaaggaatagatacagcagacctgctcaatgccactaacatattagtgtgctccacctgctgcaggcgagtgttcgactgttgtacgtaggctctagttactccaaggacagacatccctatacagtctggtcccaacagcgtccccaaatcccccctcatttcccctcacgacgggctttacgagtgagaggcgcgagggttcggggggcgccatgtttttggggaaccccagattgatcgacgattgctcaagtctgggcgggaatgaggttagttacgcggtctgaaatcccgataccagcaggagcccatgaaacaggggtatttggggggaaaagacagccgggttcgggtaacagtcggcgtaatattacaggccagtgacctcatcgtcagctgccccgacgctaaaccggaagcagagtttccattacccaacgcgaatatcatgagtggcagaaagatgtcttggaccagccagcaaaagggcgtgtttcccttctttccagggcatcaatggccatctccaactcagcgctgcattcctgccctctgcccgctctcttccgcggagatcctcgtggttacaatgtatgagtcctcgttttgcgtgccgacttttccaccgtcttctcagtgctggccggatggccccattctagtattttaatctcgaggaaccccataatggtcctgtccactcgctcttcgtctgtttcagacctccaatgggattctttggtttatatttagcttctctctcccctcctgtcaatccacgtagttcaaggtaggttctgtcctcgcgggcccccctgatatggacccgtcggggctccatttaatcaaactgatgcctggtcgcttgatcggtgtcaatgcatg

SEQ ID NO: 34 - DNA, 1500 bp, Aspergillus niger
ttttccaattttagcccttgaccaccaatcacgttggtttcatgcggtatttttcacacttgcctcattcgcactaagatggtccgcgatgccgaaaaaggaaactgagacccactcaacttggcatcgtgaatcgagtcatccgccgtggagccgttgcacaggcgggccagtatcaatgagggaaagggaaagggaaaaagagggaaaagaagggaacatacatgggaataaggcaaggagaaaatagatttttcatggatcaaaggtagcactttatatacacagtttgattacccctcgtacaagccgcatcgaagctgtactacgtagtatctagccaccgtggtatcttgtgtaggaacaaaacatcaacttgacagttggagacaacaacggcaacgtggagacccatgcacaagggcgggccatccatgatcctcaggcagaaacagccaggccgtccaacgacttgccccggaccgggccccgttcctggccgatcgcatagcaccggctttctgggtttggctggctggccgatagtatttggcttcccaaagaagtagtattgcaggtatgacttaataatggctgtgccattcggattaatctcattttgggaaatttcgatttttatttttcatttttttttttttccccttcgatgtgataataagaaatgtttattatttaattattattatttgttttacctttggggaaatacgattcctcgtctggcttcagctccagtttgtataaagagccagcatctcccgggtggatttgcacctgattccactgctttgttcgtgtccatcacattgaatatcctccctgtgatttaatttgacctgatctgatttgaattcgcatccttctttccttcttggataaattgggattctctccgctatttttccctcttctttcccaccttcccctcttcatctgtgtttcttccatctttgaatcccaaactctcccaaactagcctctttgtgcattccattggtgacgtgtcggttgatggctggtaccgacaagtgaaccccgtttcagtctgtttggctcgtttctcttgctctccccgataagcttcgagctttcgtcattgccctggcccgtagcaacgccagatcaccaattttcctttgcatcaatcgacgcttccattgaggtaggtttgtcgctagtggtctagaattcccatactttcttttctggcttccttctatatctccctgcgccggcgccatctcctgcacatcttcatgcagctgtgtggctgccttatctttcccagcttcagcttcccatcctcttcccccctccattctcgcgcacgaaccagtttcttttattgctagcagcttggctcctgccttgttactcatgcatggctcaccaagtaacagatataaacccactagtgccgcctcttttctgagttcaattgagcctctcaactcctcttgccactccactcccatcatg

SEQ ID NO: 35 - DNA, 1500 bp, Aspergillus niger
cttggcctcgcgcgatccgcttggtgaggaacggtgttattgacttgaagaagcttgtgacacaccggttcctcttggaggatgcgatcaaggccttcgaaacggctgccaaccccaagacgggagccatcaaggttcaaatcatgagctccgaagacgatgtgaaggctgcttccgctggtcagaagatttaaacagtcgtacattcgtgagcacatgcctccactgctttatattgggtgactggtcaccttggatacctttcgcattcatgacacatatttttcatgacattttgatggatggtttatcgtgtaacgttccccttttttatgacgttgttagagatccctgctggttggaggcatagagatgcacgtagaattctattccttcatttttgactctcacaacatctgcatccgacatatcgcaggtagagaagctgtctctcatcgcattgacaagcctttgcatagaatgaagcggcagagtaacccagagctgcagcttgactgaacactttgtcttaacgcatctgcacacttcccccaggtccccaagcttctgccagggtacccggcctgctggaggagtgaatatgcacacctcgagtgccacattaattagtacgatgtggtcaataccttatgccacctcattgcttaatgcttgcagcactggcagtaagcgaccgcctcttgacaggtcagagccacctatcagctacaactacgtatcagcagaccggatccgttgcatccgacgctttgtctgactcttgtgcggtttttgagtgaccagaatatttactttggtccatgttctttgagtgagatctgatcctgatcctttgaatgtctcagtttgtttgtttgcggggcgtcaggcggggcacgtggtggggagagtgaggagagccaccatcactgtcacttcctcgattccatctccatactactattcgctaccaaaagcttacttgcataaaatttggagccaatcatccagggcattaccgagagatataccgattagacgtgcccagagtctaggctgctgcacgagattcaatgaacgagaatcggtgtaagtgacctgatgttatttggtccctgcatcagccccaagccgatagcggcgaagatccccgataattgaccgagatgggacgaccttagactcacattgtcttctttaggcaatcgtctccacgtttctcggcttttctatcctataaatattgctttttgtttttcctcatagaactgctcggctcatcccgcctctttgtcagatacattccttggcttcgctgattgaatctgcggggtccggtcataccgcgcaacgccacattatgcacttcggccaacgcgccatgcattcaatgtcatcagtccgtgcccaaacacatataagccgctgggaccacccagctgggatatgaagtcacggcttgctgtaatccggggtgatcccagagccaacatcataatg

SEQ ID NO: 36 - DNA, 1500 bp, Aspergillus niger
tcaggtcttcctgaagtttatctcttctttcgcggctactgacgtcagtctccttgcgaagcgactcgagctcaatttggatggtggtaatatcgcggtcgagctctctgatcacatcgggcttcgattcttgctgtagtcgcaaggcggacgccgcctcgtctaccagatcaatcgccttatcgggaaggaaccgatccgtgatgtaccggttgctgtaagttgccgccgccaccaaagcaccatccgtgattcggacaccatggtgcacttcatacttgttcttgataccacgcaagatggatatagtagcagcaaccgagggctccccgacttggataggctggaagcgtctagccaaagccacgtccttctcaatcaacctgtactcgttcaaagtcgtggcaccgcaacattgcagctcaccgcgagacaaagccggtttcagaaggttgctggcgtcgatactgccttccgcctttcccaagccaagtaaggtatgaagctcatcaatgaagaggattacacccccttgcgcatcctcgacctccttaagaacagatttcaaccgctcttcaaaatcacctctgaacttagcaccagcaatgagagaacccagatccagcgcaataacacgtttgtccttgatgctctctggaacgtctccctgaacaatcctctgtgccaaaccctccaagaccgcagtttttccggtacccgcagctccaatcagaacggggttgtttttcgttcgtctggagagaacctgaatggtgcgatggatttcggcatcacgtccaatgaccgggtccagcttgcctgccttagccctggctgtaaggtcgacaccgaactgttccaaggctgacttttctggttcaccaccaaggttcatgcgatgtgtgcctccgggaggatgagggcggccattggcataagtgcgtataggcaagacggacgggtgccgggcgagattcgcgatccgggtgcgcgcaagtagacgcaatgcttgagaaggttgaagaacgcggttcgatgacggtagggaggatggccggagaagccgaccccgagcgagttgtaaccttgacgacatggtcaaaacaccgaccgtatgcacagttgggtgtgaaaacaaatggcacaacactgcagatcttcagtcgagctcccgatatagctgggggaaacctatatacctcgccgggacaggcggccagccgattgcctctcaggcgtcgcgaggcttctcgaaggctccaccgcacggcggacggtgagtggcggaagctcccccaagagggccagctagctccgtcaccgtggggtcacatgaccggccggaatgaggggctcacttggtgccgccgaaaaatttcactactcggaccctccgctgaaaaaatacttcagaacacctttcccctcctatttattcctccttttcttcttcttttcttctactcaccacacacatcccccaaacacacattcacaatg

SEQ ID NO: 37 - DNA, 1500 bp, Aspergillus niger
cagccttgtgggtccggcatcacaggtccattgttgagtcacgaagcctactctgtacaagccatgccatctgaaagaaaagacagagagggagggatggataaccacggaaagggaggagcaggtgactgctgaaatccgaggaggggagggatgatctcatcctgccgaccgttccgggatgcgaggatctgcagagttgcaaagcacggcggaagatttagtctcactgtagctcgaagcttctcgagagagggagggagtgcattgactgattattgatcccgttggaacccgccgggttgcacgatgggacgtggcgtggtcgccattggtctgtgccgaagctggtggaagctgcttgatgaccagcgctgagtacggtgctgagtcacggtgaaatcatagttagttaacattcctggagataattattcagtatgagtgaaaagactagatgcttctgatgatggaatagatgatagagatgggatggacagtgagtagtgatgatggaaaattgtagaaaggtagccagaattgaagaatgaagataagaccgaaggaaatccgacgagatccagaccgggcgatcccccgtgacgccaagccatgacgatggaacttgagtggcttcacggcttcaattctggcttcttccaccctccaatccttcaatcaccgtccagtcacgcaacctggcaatcatgagttgaccaacaacaccctctctcccttgcctggtccgttcccgactctgtccatctggctccatccatcagtccatggtccacgctgatcctcacactagccatgccggacctgatccccaccccaactgtgatgcagacccagctgcgttgttgcggttgttgatgttgatgctgctgtgatggccattgccttccacctcggttgggcacccctcccatcctaggaacagtatgatgctgtcccctaccttagtacccctctgttgatgtggtcttcctgcacaactacggcaatctggtcagtgcaatcacacaaccgacacaacaactggtccgcctcggaactgacttacttcatgcagaggaaattatgacgcatctggggaaaagcgccaaaaaacccgcctaagctggccctccttccgttggcgtccctcagttttccctggacaggccactactactgagggccagaaaaactttcggctcaagcttaagggggaaaaaaaaaaaatacaacaacctctgacgtaaaagatacgccccgttaattcggtcggccatgcgcccttccgtcctatccattcaagccgaccaggcgacatataaagaaccttgtcctggcattccttcccctcctcatcatccattcatcagagtccgacgaaccagtctgtcagtggaactgagcttctctttgcgcgtcgcccaataaactactagctgccctactatctcactttcttcccatctctatacactttcagtccagtcaatcgcagccatg

SEQ ID NO: 38 - DNA, 1500 bp, Aspergillus niger
ttggatggaggctgaccatgctgcagagatggatgaacttttaccgatttaatgacggtgtttaattggtcacattggacaattgcaatgattgatttaatctaaacataattcttatgacagacatgacatgagccttccatctgcttactcccatccatcactctgctttctgctctcatctactccgtagtagaaatatcaaacgtacaatgacagatcctaaacccttggtcagctaccacacttccagtgcagaacgatacctactgctgatcatcacagaatcctcctgtccacccaccaccctgttcaatgcactaacccttgaattagtctctctccatgacactggacaaaagcaacaagcagcctaccgtattccgacaggcaagattggcaatgctatccccgccacggatctggcacgaggtggaattgctcagcatgatttccatgagggcagccagggcgggcagacccaagccgaggcaggagtagagggtgactagggcggttttcatcgtggtgtagatagcttctagttggtggctaagaactagtggatgttggcggtggtggatgtctaccagagagtgagagccccttgctacgataggcctactaggtaagttctgggagggaccttggttgactggctagcaggatggctgcattctgtgagggatctgcagaggggattacttggattgaagctcgtgcatggctgcggatggtccggtgctacacagcacgatctgctatcgatatggcgagggatactcgggatttgacttgttgatctttcaatctttggatgccttaattgctcgtgcattatcttcatgcccaaaagactctgttcctgtttattcgatcaaagtcactagagttatggctgcttcgacgtggcatcccttgctgacggggccttaggtaagtaaagaggggagggggccagatgcaccttgaggaatgggtgggaggaatctcattctccgaaatccgatggtcggcaaacccccaagattggggcagagtgggatccgccggtcgcgatggataccggaaaacaatagtcatcctccagactgcggttagtgaatagttgactatggaggagatggttagattagttagttatttacatgggttaagttccgagaccgagggggatggaccaacagcagcaatcaggagaggcaccgtcagcacgccgcccggaagcacagaaagtccgaccgcatcagccccgccggccgagctttgctccgtttgaatccttcactctcttcccctctcattctctctccgcccttttatttctcttcttccccctctacttttattcccttccactctccatctttcctcacttatcctcctccccccttttctctgtaagccatctctctctctctatttttctcactcttctcactgtcagtttggctgtgtctaaccattgcatctctagtatcttcaccacacacaaccatg

SEQ ID NO: 39 - DNA, 1500 bp, Aspergillus niger
ttttcggtagaatccacttcgtcttcaggaagcacttcgaggaatcttcgtgtcagcgtctgtcataggttctcattggaagagacatgacttgattgtatggcacattctcgcccgctattttgaaggatgggtagcccttgcatgatgctgatggttgcacatgataagcatgtgtctatggttacatctgtctgttgcctcttaaagaggggttgaaggtcaataaggaacagacaaccgacgccactggattggggaagtcatacgacgctaccacctgtgacacgcggctccaagacgtagaagtcaaggttcgatatgtgaggggctattagtgattcttcactaacttcataggtgacacttcaatgtccaatcatggcaaccacgggatttcgacagtcataataccaaacggttggtggaaggacatgctggacagagtaaccgtaaaggagtcttaactgggctgcgtgatgtgaaccgagtcaagtctttcattgaaccaagatgccatacgtggaggtcacccgccaacagattcttcgaccaggctagcattgtacgtctcaggataatgcagtcaggatcctcgcgtaagcgggcgtatccttatgctcgtgataaccttctacgaccatctgcaaggggtaggacgcaccccgctcttcgccagcggggttggaatgcatcaaaggttgtttctgttgccctcgcatagtgcttttgtccgaaatatttcggtatgactcacatgaagagtatggtgaggctgtgtatttggcgagatgaaagtactgactagtgaacgggtagtgaaaatgtaaaggactgcaaatggatgtcaaatacttgccatagatgtgttaagatcttgcttggaatgtccataccaccttatacatattcatttgcaagttgctatatcatgctaaacagcctgattcacgcttgccatgggtgtatgtctgtccttcagactcttccaaaacatctaccgtgataatagactagtctatattcgttctgcaagagatcaagattttgaaggtagcacaaggtagggtttgcaaatagtgtctgtaggccatcctgcatgaagagcgcttcagagcaatctagaccttcttcgtagtcttaaaaacctccgagcatagtcgaagttgagtcagttgacaaaatacatggcaatagccaacgcaagtataggctgcaccaagccaatgttttgcaggttatcctccacatcccgcctgtaatgagaggctagatggtctatcggattactggatcaacattacgtctcaagtatgcaaatagggcgtatatcccaattagcaagtcgaaagaattatctaaattgggaccgaactcaagggtgaaagggatatataaaccatcgaaagtcccattcttcctcctgaagttttctatcatcccaacacactgcacattatcacatctcaagaaaaaaaaaaattcagacccgtcacaatg

SEQ ID NO: 40 - DNA, 1500 bp, Aspergillus niger
aaaccgttgtcgcacaaattccatccatccatgatgagagctaatcatagcgattaagttcggacgtgcatttctggaaacatggtggaatcggtccgtgggtggtaaacgatgcgcatatcttggggcatgagtctgcaggcctcgtcgtcaaggtccacccttctgtcacgacactcgcagtgggagatcgcgtcgcgatcgaacctcacattgtatgcaaggcatgcgaaccgtgtcttaccggacgctacaatgggtgcaagaatctgcaattccgctcaagtccaccatcgcatggtctcctgcggacctacgtgaaccatccagccatctggtgccacacaatcggcgatctatcatatcagaaaggcgccttgttggagccactaagtgtcgcactcaccgcagttactcgatctggggtcagaattggagatccggttttgatttgtggcgcgggaccgattgggttggtggtgctgcaatgctgccgcgctgctggggcgtatcccactgtgatcagcgatgttaacccggcgcggttgcaatttgcgcgacagttcgtgcctgctgctcgaacactgcaggttcggccggaggagaccgacaaggaatttgcagctcgggtggtgcagttgatgggtggggaggattgcgagcccgtggttgccctggaatgcacgggagtcgaaagttcgatcgccggtgctattcagagtgtcaagtttgggggcaaagtgtttgtggttggcgtgggcaaggataagcttcagtttccattcatgcgactgagcgagcgcgagatcgatctgcagttccagcagcgctatgtcaacatgtggccccgcgccatccgggtgatgtccagcggagccatcgatctagaccagatgatcactcatgtctatgcctttgaagacgctctcaaggcgtttcagacggcgtcagatccgagtagcggctcaattaaggtgctgattgaggggccacaaggatgagagatggcggatattggacagtgaagccatgactgttataaggtatagcagaacagaacatgtatcttgtcaacgaattgattgatttcagcacggaaatgttcaaggcatggaacaattgcttctgacccggaaccgcggattgcaaggatggatatgtggatcagatggtgtgaagaagatctgtctgttagcatacttcataagttccaaggaggagtaaatgtggagtcataagagtaatatcgaatatcatagaatggggttgtcttgctaacggtgaatcggactccgtcggctctgccagcgatcggcaggtcacgcgtctgcgtaacttggtacttatcttcccagtcacctaagcactgttactccactcttctctctttgacgataagaagatagcagagactcttgataaaaagggttgggagcatgctttgaatttcttcactccgagagctttctgtccccatctggtctcttgttgtccccgatg

SEQ ID NO: 41 - DNA, 1500 bp, Aspergillus niger
tttgccctaactattctctatatggagtgcgccctccaccgattatcatataaataatgaaagtgaataaatgaaagtgatcaattgatttataatgggtccttccattttatctcgtagcggcaccactaaggatataggcaacctcatcgctcttttcggccggcccgaactcctcagctgggtgacggtcagtactcgtacaccaataaattcaatcctgataaaatcacatagaagggaaactatcttcaatcgaaccagaacgattgttagccccgtcttaccagctagatcacaaactaccatgcaagctctgtcaggagtaaatatgcatgacccatggatctagtctgcagcaaatcaaggacaatcaggagtcaaaaattctccagctcaaacagttgcgcttctaactctacgaagtgaattcaattgagaccggtttaatcctcggcgacggcgtggaaaagtccaattagcttgcaaggatgaagatggcatatgcatacgctgagatactaacagaaagaattggcagtaatgatcacaaataacattaggaagcgaggtctaaaacagactatctggcactgagggaatggctccagaaagctgaaactgggttgggtagctgttaatacatgcagatgggatatttacactctgctcgacgatggcgcgccatgtgttcttcccctatctcagtcccataacaacttccagggcggccgctgaattcaataaagccgctaattgctcagaaatggagaaggtgctcgggatggataagttggctctaatgaatgatgagtttgaatggcaaatgatcgataaagaggactctttgctatcagactagctggcacggaatagatactcgtctccactagggttgggaacagtgagcgaaatgtgggccgctaatgtgccacggcgtggataaaccaatcgtcctacatccccaatggaagccaacaccaaaaccggctaaaccctacaataccatactgctggaaatcaaactataattcacacgggtaacccagcaagtttaaacaaggatgataccccgcagacgacttctcccaggctatcggaagtgggatagtgtgcgttcgccccaagtggaacggaagccgacatggacttttcggatttgcattccggatcggcaatccaagaagggcaaccgaaaccccatcgaaacctggcagcgaaaccatgacaccttcgaaggcatgggcttccgttctctggcggatctggcggctaatcggttgacctgtgtacatggaccacatcggtagtaccgacctgcacaggtgaccgactgatgcgtctggctctaacgtaccgtccgtgccacatatcatatatcgccattatacctcatgaccatgacaaaatatcttgttgcgctgctcattgtcgtcggcacttttcacatattaatcaattatcgcgttacacgtctctatcgctcgcctgctcaccatg

SEQ ID NO: 42 - DNA, 1500 bp, Aspergillus niger
tccatgctatatcctacactgcgccacgtggtagcggcctaaggtcgctgcgaacgcgactcagacaactatatgaactatcgtctgtattactacgtctgtcgtttcggttatatgtgctcatggatccctggcgggttggtaggtacccgtacggcatccgaaaaagcaaaatgaggaagtctaggcaccaacaacttccggagaaatatcggtgctttacccagcttcctcacgcggaggaagcccgaagaagggcccaaacaaagcgggattgaacacggttagccgagaactcaactgactttacagaagcctagcaagtggggccccgaagtaggtacgagagagattacgacgcaagttagacccttcgccacctctccccccggggaagtaccgtcgctgcgagattaatcgcgcatagggtgctgagacaccccgtggagaagggttgtctggtccggggcaatgggctgctggcgatacgggccgaattacgtattattgcctcgcggacatattgacgctctccgcaagaatgaaattgagctgctgtggaatgaactcacgcgggattctttgcaacaacgtcatactcgagatcccttttgctaatctaaatcttcgcttttctctaggcctagggatcttcgagaaacttgaatacgaccgagaaacgcccagtcgttttgaccccatgggaggcacacctaatgtacgaaggtatcgcctagtatttctcaaatgcttcgagtagtctaaccattacatattgtggcagagtacggagtacttagtgtgctaccagttccgaccgggcagtctccggagaaaattactcgggtcccagtcaaccccggtcctgctgagccgtccgacttggcaattgggtgtgagcccgtaaaactttgcccgtgcacgttccttccgagcggggaagggcatggaagtttctttgctaaacggactcagagtattgggggcgtcgctcacttgctactactcaatcaagttaggcatgtttggccttgagggtttccgggggtcctgtatttctaacgacccgacttctgttagctgttcaagaacccttgacgtcgagcctttcacgctcgctgcgattaaaagaaccttttctccatggcgtgggttggatttgatagtcgttaggcgcgttgcttttcttattcacatcaccccataataatacaaacctgatacaccttgatgcaaccgcgtttgggtccagaagagaaagaaaagacttgattgtcggatactctggggacttgtggggtcgaggaagtggggtctgggacatggaggccggggattggagagagggacaataatggtacggagtacaataatccgcagacaaccttcccggctaaacaggcattattatatagagggcattggggtcgtctgactatttttcttccaccctgaccacacataatccttttatcatattgattcgacaaactcgaaatg

SEQ ID NO: 43 - DNA, 1500 bp, Aspergillus niger
gacacaagccgaggatgaccacatcatagtaatctacctaagtggcaaggcgggatatggtattcctatccacggtagcaatgtacgaaaatgtttctctctggctgaaccaaacccatttcctgaaaagagagtttgggtaggaaaatcttcggggacatgtagactatagtggcggtcaccacgcacaccaaggtccctgaggatatttgcaggtggatgggttacgtattggcttacttggcagaggaagagcagggcggtgatgtgtggagaccagtgattggcttgatattggcctagatggggctgttttctcgtgattcgggattttagaccaataggaggcgagtcttggtgctgggatggaggggcactgcatgtatgcctgaggtgtcgaggtgggatggtttcctcagtatcatctttaaaaggcagagataagcttgttggcggtgatataacggagggaaggggaactgggctgttgatacagctacacattaaggattcggagaattgagtggcaaaccccgttattcggagaaagaaaagcagttctgtgtccatttcggatttacggctgacctctcctttggttctgcgaataggcttgatatgcagtattgattgattcttagttgccaatggctatcccctaagcatattgatagaaaatccatctgaattgggaaccaggttggaggtggagtggcgtcaggatcaggggttgtacgagaagtaaccaagctttggttgtaccgatgcagactcggacgaagcgccggcttggttggattgactgtctctgataactagatctgccagttgataccacgccgcaagaggataatgagcctcgggaattgatactctactgatagcagtgctacttggctgcagaattgatgcccagaagtcatgagcgtgatttgtgatggcccagagatgcaagccagatagacccatgattgaaccctgcagagagagccgacctagtacgggatatctgatccggaaacccattccaggaagaagcagcaagacatgaacatcgcctcgagggccgatttgtcaacggggacggtctagatatggtggccagcggacgggaatggcgcaggcccgaaaaaggaatgcgtggtggggtcagtcagtctcgggcggtcttcttgataatacagttccccctccaagccctaccaatcgaagagtttcggtatccttctgcttcggatcacctcatggcgccgtgggcgggccgtcgccgcatcattcgttgcccttgacacaggaaccattcgctgaactgaagctaaggtcgaattacccgccacaggctcgttgccccgttccgtctccccttcttccctatataaccaaccttccattcccttcccaaacttcttcactctcacaactcattactactactactactactcaatcactcttaacctttctagtacacgttactaccaactaatctcattcacaatg

SEQ ID NO: 44 - DNA, 1500 bp, Aspergillus niger
gctgctcgaccggaggctctgtgacataagtcgtgtcttaatcacccaaattggattcgtcgcaagggtcgagcaagcgcccgccgttatggaggcgtaacctcgtgagagccaccaactatctgttccactgtcagcctcaccatcacccattctccgtagcatcactcctcacctgttgtttcgtagaaatactcccgagaccgatcgtagaccgccaaatagacggcccacgtcggcaggtatcccaggagcatgggacccaatccttgataaaggcctcgaataccatcctcccgccatattactcgtccagttcctagcatgccccggtacagtgttttggcctcgaccgccttgccacgtcgtcgcgcgaacccaccctgagcctgcagcttggtcttgatcacatcgagagggcaggtgacaattcccgaggctacgcccgcactggccccgcagaacggcgtgatgtaataatccggcacacgtgtagcgagcatttcgaagcgatcgagaagagtgggctgggatggggactcggccgatacatgaagcgtcgacgaaggcgaactgtccgaagttgtaccctgctgctcttgagagccgctggcagcccccgaagggccatggccttcagagaacatctttccagccagcactcaaagcagtatgcaatccgtctactatgtcaccgagggttcccggatccgagcatatcggtgctcgctccggaacccaagaataacccgtgtgtcaaatcgtggtgagaggggaggaaagcgtgacgtcgttcggggcgctggtagaacaggaaaaactaccccgagacaatgaattgaagggttttttctttcgcagagaggagccgatgctgccgggaggagtgggcgtaataaggttggcctatctgaagcacagacggggcggggtttgtaggagcaacgcagctcccctatcggagtgaaagggaatcgaactaagaaaaatgttcaacggatagataataagcggcatgcccagtgagtacaaaccaaagtatcagaaacaaagaccgtcggggtgaagtccgaacagatgggagcgccggtgttaagaggaggcagacggaacaggacggagcaatgaggacgggatgaattgagctggagggattgtcttagtcgtattccagtcagtcaggtggagtggagtcgggaatgagtgagtgaatcttgtccgcctccaagcgcgccgctcttctggcctgaggcaaccatggcgcaagctgagccggtcacgtggaaaccagtcttacaaagtaacttctcggaatgattcctcctgcctcaggcaccaacatcggatgtttttcggaaagtcacccgccgaacctccgttttgcacaatctccaacgtcgggccatctcctgctcagcttgactgacatttcgcgatcctccccctccacaccaccccgtcaattggacccgaacaaccccgctgggatcaatcaccatg

SEQ ID NO: 45 - DNA, 1500 bp, Aspergillus niger
ggactgatggctccagtataagaagctccgccctatgcacctattggtgcctgaggtctcacatacttttaagcgaactgaacaaactaggattccgtatgaatataagaaatagctggaaggctatctcatcttgttcaaaggcacgttcgattagcacgatctatccgtcctagatatggcagggcttgctcgaaaagctcgggatcgcccggctcggcttgtgccaagatgacccgagcctgcttatttaaggcttgcgatttgatactattggtgctcaagagtatgttgatactcaaaagtttggctcaaatataaaagaatcattaaaataaggctcaatattttataaggaccaggataatgatggacaacccggctggacagaagtacaatacttgatattgttagtgcacagtttatttagatcacatagctagattatgtaagcttagacaagcgctcagaataacagtcactttctctaatatattctcctggggcaatccaagatcattcagggattgctcaaaacatatacttttgtacttctcttatactccaggccactcagtgaacaagaagcagcgccacgttcaacctaaagtggaaaggggaagtcactcaattttcgcagaaatccaattcagtagcagtacggctagttgcaatatatataccaagcagcacaaccattgagaacaagttcttactactgaagctatcggacaatccagcagtatttccagctatagtgacggcatcaaaccatttacttattctatacttcatctaagcaattgttcttattagcaattggctatctttggaacggcggtgatggcatcggagttcgcagcgagagatgggctgcttatcgcccacccctgccactgataagcgatgcagatctgatatgttctcaacacattgaccagtcgcatcttatggctattgccactccaccgtcatcggcagccggcggaagaggcaagcagattgcatcaacagagaacatcttcatctgaatgattggattatggaattcctattatcattgggaattatcatgctgaacggcgactctccggatgggttggcaagtcgccgagcacggggccaacacgatcgctgattggtgcaaaacgccgaagcagctgtacggcgcggggcagcttccaatccatcgcgagctctcctttaacagctgccccctcgcttccatttaaaagcccttcgcgcggccatttttctcgtgctttatcttctctccccatagtcatctcaattgcaccagttgaagctataatcggtgctggtttgctgcttgtctggttgagcatcgatcgaggagctccttatcaatcatcattgcgttatcgtcatcgtatccttccccacatccctgatttccagctgggtatttctgtgcccctggccaccccaactcattcctttttctcagacccatctcctctccattaattcatcacaatg

SEQ ID NO: 46 - DNA, 1500 bp, Aspergillus niger
ccaggagggttctgactaagttgttgcatggctgcgggaggcagggcgatagtaatattagactgcgttaatattatgcaggcgtggctttttactgcacccggccatcagtgaatcaagggagggtggatcagccaatgtcgcccccctccttccaaacaaacaaatcattcacgttgtcaagatgcttcccttcctttctgtgacacggaatggccggagacagcgatgttttagccactgggattgcagtatgtggtggttccgggtaacatcgacatgtcagtgaatcaatcaatgatgcaaaccgttttggtcgtacgtaaattgtttgtctggtgacgggaatcggtctgccaacccgacgacagccgcatcatagttataattattttagattatgattcggttgaaggaggactcggtaccattcatcatcgtcatcatcagcaatcatcatcaaagatcaatgctctccttcgggatcagtgtcacggtcacactgtggcaaaacattctgtgtagccgatctggagattcctactctatacaaggatagatgaaatcgatggacaataacaacaacaacaacatcatcatcatcatcatcatcatcatcacaaaggagctgtatacaaagagtatagtatgcatgtatgcatgtatgtatgtatttacgtgggatacagaatgtgtagagaatcagttaggattcggaaagtcacgactcaagaatcctccgaaagaggcaagaatgttggtaacaaaataacagcaaaaaaagaaagaaggcaaaaagagcaggtgatgccctcgtgacctgcccgcatccatcatcccaaatcctcccgtcaggaactccagcttcatccactccattctttcttagcgctactccatccaatctccagtctctctcctccttccctccatctcggttcgtctccatccgctttttctccgccggcgaaaacggattccccccgattcaggtactcaccgtccacatctccccgtttgtcgtagcctgctccggctctctccagacagtctctccactctctccccactccaagacacgctctcgaagcatccaatgaactctccgccccctagcttccgaatctcccacctcgttcgcctgtcttcgggcccttgagatctgggcccccgttttttcttgccatggtcctaccccgaggatggggtcctccaatccctgggatggaggcgaatcgccgacggatggagcgtcaaattctttcacttccattttagtggcattcactcgttgacaccgtcatacttaccgagttcatctttgtgtgcggcccccctccccccccctctccccctctgcccggaccccatatatatccctccatctcccccgggtctgtcagccctttggatgtctttctctcccatcaatcttctctttttcccatcttcccatccctggcagaggattccgcgttgtatgaatccaccggcaacatg

SEQ ID NO: 47 - DNA, 1500 bp, Aspergillus niger
gaatatacctcaaaccagaggtgccatatggtccgggcgagacctgtcccaaccagatgaaagtggcacgacgtgttcaacggagttgtcatggccccggtccataatttagattcaatgctactggggccatctgataacgtgggggtccagtgagattgatcccttgtctggggaatgatttcaagacttgcggtgtgtgggacgcccttgcttggatgggttggcatgtcccaaccaagcttggatcatgtggttgcctgaggcgataagagtcaatgggaggaatgtaacatgggtcacgtggtgcaccgttgtggaaagtgttgggggtgaatgcatgcctgaatcatgtggcgacgatcatcaggttttcattctccggactattcaaaggagcatcaacggagcacccggaaacaatttgtgccgaggagcttgcggctcgcagtcatgccaaaacggaatttgatgtttcaaactcggccggtggcaataatgtccactcatagggctgccctgtgcggaaaagtctgatctgagtggaccaatgactgatccaaggcatgctgattatagcactagaatgcgttagtgtggccacagcataagcaatgactccaatgtttcaattctccagaattaggatcaactctggaagaaatagggactattgctcgagtcgcaagctagatggagtggtgctactccgagcaggcaatactttgatgcggaaaaggaaaccgtcccgcaatcccaatcgggatggatagccacagtcaagccacccgagcaatgacaccagccacacagagcgatcaaggggcaaaaaacgttggggattcaacgaatggttgaactgttctgattggtggtccgctcccgaccttacccaaagcggcagcttctggccgagcagcgccatcgaatcagagggagcccaacaagcttagttggaggaacaggcggcgctgtatggttggagatactccggccatttgccatcgcggatacactctgccatccggacaccttccagacgtgcctggataatactgtggtagtagtccccttcctcacgctccctttcttgtgtctgttgaaccgtcggccactttgggacctcgggatctcatgattacttcactgatctaccagtgaacttgccgtcaggccacccttcttaacttattccatgcgggtgtcctcatagtcgcatcattatcattgattgtccgccttgcttttccccaaccatcatccgccggtggaccctggtagagttcaactgcctccgaattttccctccttcactggctcagatctgccgcttacttcttcggcctttcgattgcattccccaccctttttctccgtctcttttttatcgctcgcctccggctcttctctttaaacccaccctctcccgcacgcatccttctcctcctcatccgaaccatactgcaggggtcgacaatacacacgaaggacatcaccatg

SEQ ID NO: 48 - DNA, 1500 bp, Aspergillus niger
ggaggggctcctcctcgcgatctgtccaagctcggcatggtcgatctcatgcaagaaaaggaacgcatcgaagaagaactctcggctctgagtagcttccttggctcggtgagttgaactgatattggataagccttgccactctgtgagatgtcgactgacgatgctttctgtctagcatggcgttaatatgaacacatctctcactactttcgatggcttcccacgagacgatatcgatgttgcccaaagtgagtcactactttttgcctccgcagtgcgtctgctaaacagtagttagtacgcaccactcgagcccggattattcgactgcggaatgaccataaagacgtgatgtcacatctggagaaaggaattcacaaccactttgccaatctgcagcgggcgcaaactgccgcacagtcgggtggcttggggtcgcaacctagtgtgactgggaataatacctccggcactggggcatctgggttacccttcgcaaaggtcaatagtgtggtgcctggaagtcccgcagaccaggcaggtctaagggttggagataccgttcgggagttcggaagtgccaactggctcaaccatgaacgtctctccagggtggcggagatagtgcagcagagtgaaggggtaagtaggcgatgtgcttccaacctcttagtcgtctagtccagggacatgcactcgggagaacgctctaggctaacgtcttctagcgcactgtggcggtcaaagtcgtgaggaaagatccgagttcttccagcagcatcgacttgtccctgcagcttgtccctcgccgtgattggggcggtcggggcctgttgggctgtcatctcgtgccgctgtagtggactggcatcgggttaggcacgaggatgacggccgtttactgtactctgggatagtccctagaatactaggacttagccgggtacgatctactttcgtttatgatttctcccttttgcttgctattggactttatgaggaatgtgacatgtattcatgatcgaacgcgacgtgtttattcttgcagggaaatacctagggaagcattcagggtggcgtttcagtttaggttcaagagatgaaattgcacctgtttatatggacattgtctatcagttccgtacagcgtagagcgttcactcgcgaagaaccgcgtggatcgacagcgaggcatgcaataaatgcagtttgcgcgatcctgcggggcacgtggctgcctgaggcccaaaaagatcacgtggccgtcaggcttgggcgaccgactcacggcaaaaattccatcaccggtagaaataaccttcccactctaactcctccctgtccaaccccctcgttctatcgaactcccctcggtcgcttcgtccatcgagtgtcgcatctcactgtcgacttttaatttccgtcgacttctatcgcagactctgtatcgcttctcttcatctccaacccctcacatcacatccaaccttcatcatg

SEQ ID NO: 49 - DNA, 1500 bp, Aspergillus niger
ctatagagccgctcatcctgctggcacgaagctttcgggaaggcatcattcttttgtctgccgatcactcctggatctgactcaggtttttgtctcccagcaaacccattgcaagttatgcgacacggagttcattccatcatgcagcggacggtgtctctgaaaagaagccctaaatgagcacgtcatggctcctcatgactgggatatggtaccagataaatttgtgctggggtctgacgttggagcgaaggcatcgtccgttgagccgtacatgacattctatgactggttgccatagttgttagtccaggtatttgatcatacatggaaactcggcaaacacaagatagaaactggcaacttgtatgataagatgcgtttccccagcgggcatccatggcaggtttagaatgaggcgtatatctacggtccctccataaagtgcggataaacaggcctgtcacagagctcctcttccacgaatggcctattagatccagcaaaatgagccaagaatagtagtgtcctcctccaactctcttagattgggattatcaacattgggcatactggagtcgctgtcggccgtgatttgggcctggcgggtaatgcgacatgttagattggcagcgatgcaggtggagcaggaagagacacggttgcatttagtctacgagagtgaaattcggttagaatgcaaatgcacctaaggttcatggggttaagacgagtgtctcaccttgcgactccggcactggtcacacagaacaccatcatcaacaaatcgatcaaatgaatcggcaatggtcaatgaggtaaggcatgctaagggatgagacgatgagagtccaactcacagccggtcttttgctgtttccacgggtgccgataccgacctccatagcgcgggacattggctggtctacacagtacgctctgtggttgcatgtgggggagggacaatcggtgcaactcgcggcggaaccggagctgccaggcagttattccagccccatccagtagatagtagtagttggtagtcagatccatgcagaccaaccgagtcaacgctagcagtgtgagagactgcctgaggcaatcattctcaatatgctcttccctctttgggagggaatgtaggagttgcctcgccatcgggatgccaactagaaaggccgagattgaggttagcgcgggacccgcgctaacccgaatctcggtcggcacatttccccatcgggcagtcctcggattcggagagggtaacacacaaagtactatgcccaacatgcttgccgaccagaaacaccacggtccgtgaccggggactagggagagacccatccaagatgatgccgaacactcgcaattccattcgcataaggtggtagagaggtataaagtcaccgggagaagtccatggtctcatagtcaaagcattccaaacccttccgaccaagtcccttgaacttcctccccattccccacatcacaatg

SEQ ID NO: 50 - DNA, 1500 bp, Aspergillus niger
cgcccttatctcatggaatttcctttttgtgggacccttgcacctgtcacccaaacattgaacaatagcaataacaacaaacgagcccatcgaacccatgagcactatccgaagcgaccctactaatgaaacctccagataaaaatggtacgtatcgagggcaagattagttccaggtgggaaaaaagagagacagggggcacaagtgggtccagcagccaaccagccattgggtcaaaagcggggaagagagagccacagcgggatagtggccagagcgatgagtggccaacggcatcaggaacagcctgcaggccccactgaccccaacactcattggacagcagggagaaacatgaaacaaagtctaagttggcattcaacgtcctgtggctcagtaaagcgaggtttctgccccctgaacttatttccttttgattatcgggggaggtggatgatgatgacatcctaatttttggacgggttacaagtagtcagtttagttgatcccgcttttaccgttgggaccgagtggtgaatgttactgcctggtagactaaagtatgtactagtaagtaaagtaccagtagtagtagtggtgatgtactacgctgtagtgacgggacgatgtgcctgactgatgcaggtacggaatcccgcgcggttcatcaaagtaaataattgctatctactattgcttgaaatccaaaaaagttcgaacctaaaaatccagtaggggaaccatcgatgacccaactaagtatctaccgaccccattaactcccccctccccccctgagccgaagagttagtcgactgttcttccggtcgaaaagagcaatggagcgaccgatcaacagcacttgtctcgcaacgcatggacagcttggacagcatggacagtaattcagggccagggacgaaagtcacttggcggcactagttggtctagtcaaactggcgaaggggctggttggaccaaccaagagctgtgggtgagacgggcgcagaacccacgaggcgcgaaaacgaaaaaaaacgagcgaaagaagtgagaagtgagaggggccatcgcgggttgccccaaacttgcccatgaaggtgctctcacaccgcatcggcgctctgattgattcgggggccatcaactccacccgcgtgcagttgcgctctccccttttcgtccctgagacgatctccaggttaatctcccggctcatacaaagggcccccggttcccccctctgcctcccagctcgttcctggttctctctgttctttctctttcaagaccagacttctttgactgcttgaagcatcccattgacttcggtcagccactcttttaaccaaaccccccaagcaaaaccccaactagccacactctcttttatctaaacgcagctccctgtgttttacatacaaaacaacccacacataaaaatcaaattatcatcgaccttgacggttttattctttcccaacacaacattcaagatg

SEQ ID NO: 51 - DNA, 1500 bp, Aspergillus niger
gaagaagcgaccggggaggacagaacccgaatgcgattggggagtctctggtctgaaaacaaaagccgcacagaagaggacacatcgacaacggccattattcatctctcactcggtccagatgcgttagctcgcagtcagatatgccgctgagctataagctagttaagctgccagcgggacaaatgtaagccaacagacaccgaaacctctggttttggcctggtcgcttgcgagattcatggcctatgatacagatcaagatcgaatccagtgctaccttgcgcaaagcaaagtgggcattccgatgcaagggaaggccaagcaaacaataaggccacgcaagaggggtaatgtgttcttccgccggggctacggtgaaagacatttcgccccataaacaccgcgccaagaggattcgccaggtcaaacagtctccatagcggggttttcctgggataagccacaccacaggtttcgatctgctgatggtagagcgttgccagccaacggtctcccgtacactcttttcccggaattattctgcctaaggcactcagccctgtcaggggcatttctaacttcttggccagcaagacaagagattcgaaacacagccactccgcccggaatcgctcagcctagaaaacgggaatgttgtcaatcagccttatgtcattatctcggctctgtctcactcgccaagggattatcccgtggggcgtcgttgcgcccagtgactgtcagcccaacagaagcccatggatgcatctgcgaccagccggatccaacagcagccgcaaatgctcatcgatgacaagcatgtcgccattccagattcaacgcacagcgggccatgacatgcaccaagcgccaataccaagactgctgcctaggacgatagggaaacggacgactgaccatggagatggagccctgcctgtggctgagatgaagggggagggaccctttcagggtccaccgctgacctgtctgccaagaagcctcattgagcacgcggagtggggggatcactggccccagagaccaccatgaaacaataattgtcaattcctatggagcaatcagccacaatagtgggaattcaggcgcctgtctggggctagaaacggtgaaccgtcaaatcaccgttcatggtgcttcactccataccggggttgttgcattgcattcccctatcccaagaagcccccacgaaaatagcctacctactcttaccaggttgtcgacaatcaatcaatgaatgctcaagccactggcaaatgacctgaggccaagtgtatgatatccatgctcaggatctcttggtgactgcgaagagccgactccactgaggcataacgaggtatataaacccccccttcatcccctcttgccctccatcaacaaacccaaccacaaccacaaagcaatcaatctcattatacaaccacttttcctccattccctcataacctctttcacaactccatcacaatg

SEQ ID NO: 52 - DNA, 1500 bp, Aspergillus niger
aagcgggcagctgagtcagccaccgcggaacggaggaattgccggccgacattcgtcaaacccacaaaaaaagagtgggtgataggttttcttttaccatcccaatctcaaaagacgaggccggagcgcacttctctcaacaaccacattaaacctttaggtatgtggtattctcccgcaagccacggctgattgcccgttattttgatcatgttcaaaaaacattttttgtcatggccaaaaaagcggtagcattcagtagtgggctctgaaatagcggagtgaacaaatgctgattaaaattgatcatgcagaagtttagtcttcagcgtaacgatggcccccaagagcaagaagaccggtgacaccatcaactcgcgcctggcgcttgtgatgaagtccggaaagggtatgttataccagaatttgccagttcagacattttagacagtctctaatcgagcccatctagtcaccctcggctacaagtccaccatcaagaccctccgcgtgagtataccggataattcaggcaatagccgtgcagaacatccgtgaatggtcgactaataacgtcaacagtccggcaaggccaagctggtcatcatcgccgccaacactcctcctcttcgcaagagtgagcttgagtactacgccatgctcgccaaggcccccgtccaccacttctccggcaacaacgtgagtgactgcgatcagatctttggcatctctatggagtggggtccggacctagttctggatagactacttccgtagtgatcgcaacgtaaaactggagtaaacgctaacattatggtgaacagatcgagctcggtaccgcctgcggtaagctcttccgcacctccaccatggctatcctcgatgctggtgactccgacatcctgtccagccagtaggtgtaaagcctagtcaatggattcatgttatgtctgaacggcggtatgccatgatgcacaacgtcacggcacgatttagataatgacaatgaactttacatctatattgggtttcgtttctaaacgctgtaggataagactgtaggagtgatctggaagatagcacggtcgcatcactaaagaaccggagggaggagtggcagctcaactccactttatgagtcacgtgattaaaagtcaggcatttagacaattataaagcagcgaaaagtcatcacattccattccgcagtaactaaaacatgtttcattcacggcttagaaagaactgatatatcagtctgaacaagaatgtcggagttgccgtagtcttacaaatgcgtcatagcggctacagaatgtgattggcggagagggtagggtttagggctgtgccactcggtagcgggcgcttgcttggaaatcaacgtggaaagtgcctgcgcccataatcccccttccctttcgcctcccgacaccacctttttcgacaacctcaccgttcgcaggaagtgcagccgccccatttccagcagtcaagatg

SEQ ID NO: 53 - DNA, 1500 bp, Aspergillus niger
tcgccccccttaggggtgggtaataattacaccattgaccatcttgattgatatcatatattggaaaacggagtgatggcaaaaaaaaaagaaaacaaaaagggtaaggggatgactgaaaacagaggggagatacgtagattttccacgttactaaaaagtaaataacgactccagcttgtgtggcccatcatccatggtatctgaggcacctcgaatccttgacacgacatctggtaggtcagtttggtaggcagcatgagccacctcaggcggcagtaagccaatcaccgaggaccctgccaagtaatccgcaattcattcgaccgacaagtcaaagaatctccaggcccgaattggtgagggctgaggcgtcggggggttgtggttgttgtggacagagttggacggcgaaaagtgggtaacgatacgcgcgttttggtactgcgtgcggtaataccgatggtaacccccgcccggtgcaggtgggatgaatgggggagggtgaagcgggcttgtgataggctgcaggggcgcccatctgcttaccatatttacaccacccgggcctttttaggaataatccagtatcgcatacaggtacaggactgggcgcgcaagtaaattagcagatccagatcccaaaataaccaccagcgccgccccccctctccccttcttcccaggtcctactagggagtagcgccagtcctatcttccctgcttgggtggtcaatcaattcccttttcaccacttaccgtccaactcaagcctcctcctccctccccgtccgtctcttagttccttccttccttcttttcgttcttttgatcttcgacctctttttcctttcggttcctgttcgctctttttttcgtcgacacttcgtttcccccgactcatcttctgcaacactagcgaccttccctccctcccttccttctttcttgctttcgtttgaggaaagaagagggaggcctttcttggctgagacaggttcagccaacggcagtcgggaggcttttgcttttgaaatcccaccccctagtcgtgttttgttctttacttcacttcacccttccattcgtttatcgaacgtccctccagcgacgaccaaccggccccgccctggaagtcgacctacagagcaagagaaaaaaataagcagacgatcgtttcgagttgagattgttgtttagttttacgcccttgaaacacactccggcactggtgccttcagagaaaaagaacgctgcgccagcatcaaaagtcattcttcgacgcgttctgaacatacacatccattgcgccagaagtgctatctgtttacgcttatttcactggaaatcggatcagagtaatttttaatgggccactgttaatgtaagttgcaggatcttgcattcaaaacgccatccgaccccttagtaaggccatattgatactcatggtttagaagaagatattaaacggttcggtgattgtggctattcaacatg

SEQ ID NO: 54 - DNA, 1500 bp, Aspergillus niger
gtaaataaaatacgacaaagcaaacccagacccatgatacttgtaataaaaagatcaagaacaccccaacgtcccagtcataccatgaaccaacccgaaaaagcaacccgtcgcgtcactgctgcggaaggacaactgacgcgccgaccgcgcaggatggctaaaaagaaacaagcaggaaaataaaaaaggcgtttaaccaccgaaaccgtagagggtacctgaggaattccatgttagacgcgttggacacacaaaagccaaaacgtgtggtacttacggccctgtctcttaagagcatagacgacgtcgagggaggtgacggtcttgcgcttggcgtgctcagtgtaggtgacagcgtcacggatgacaccctcaaggaaggtcttgaggacaccacgggtctcctcgtagatcatggcagagatacgcttgacaccaccacgacgagcgagacgacggatagcgggcttagtgataccctggatgttgtcacgcaagatcttgcggtgacgcttggcgccacccttgccgagaccctttccaccctttccacctgttgagacgcgtcaagtcagtcactgttgatcatggagtagacggcagtggacaatctatcgagatgatagatagcccatgatgggaagtgatgaacttacggccagtcattgtgaaggttgtctaaaaagtaaacggaaatagagttgaattaaagttgagggtgaagatggtggagataagttaagcgttttagagtgaggatggatgtaaagggaagagggcagagagcatccatgggacgggagatatttgtatagcagaaggcgcgccgcggggagggcgttggaatcacacgctagagcgcggatcgaccaatcagagcgcaggatcccggccggcggactgatctggcggcaacgtgaccgggcagggatgattccgctgggatgagactgattcccacgggaatgctcccaaccacccagatcagcaatcagagggtgtcagggacattcctgtcgatggccatgagcatctgcaacaggttgataggatcacattccgggcctgaggttgggatcaatgtgaggcaggggcagcttctagatccgtgatcctggcttcctattggctggcgggcattagggcgctcgcttagcgctccgagcaggcagaaaggggactgaccgcctttgggcacttttgattttcccagccaaaagtgagccccgaaatcacggcttatggtgtgctgctttccgaacactttttcgatcaaaggaacggcacgctaaggccccctccctttgctggcaaacccagatcaccacctaccctttcgcttactacaaatacctctccccacccgcctcctccatccttcttccatctcctcatcccgtctacccttatctatcaacctccatcaatctttcatcctcaacgtcatctttccatctctaaaacaactaaatccatcaaacttaatccactaagatg

SEQ ID NO: 55 - DNA, 1500 bp, Aspergillus niger
tgcgcattgatattcggagatcgtagggctcgaatttgagagaccaggtagtccctgattcgttccactgcctacgtagtttgtcagtcggtttgtaaagtgaatctatatcggaagcagtacctttttcttgatatcacccagtaaaggccgcacatcttcgatagacttgctactattggtgcctgaggccttggcctcgatgctagccgtccgagtttcaatctcattcaaggccttgacccagttctcatcaataggcccctccgccactaaccggacggtcttcggagacaaactgatttcttccacggcaggaccaagcagctgttcaacattccgccgattctcgagcatcgcattcagctgtatagaccgagattgcaaactctcaatttctgccgatacagctcccaattcattctgaaagtcattcaaatacatttcaactgacttggagacgtcatcgcagccagtgattgccgagtgcaggtcctggaatttgtccctttctttctcaaattgctggatggtctgggcaccgacatcgctttgccaagctcttcgcggttggtcttcttccgcgataaagtcctccaggctgagtccatgaaaatcaatgtcttccacgagctccgggggtttcagttccgcgacggacgctggagagctgtctcttgttttgccttgtttcccaatgatcccattcaagacttcgagcggatcagcgacatcggatgggcggggtcttgtagcactcggttttggcgaggatccttcgcctcgcgcaggcgcagagagggaggtagacgaatcattcggggtcgacaagagagacaacgctgaaccttggcgactagacgcggggcgattgttctgaggggttggagccaagcgagaagaggtccttcgaggcaagggagagctgctgcgactatcgaattggggaccggagggcgtcgcatggcccgaaatgcggtctagccacatcttgggaggccggcacggagaagcgtgcggatagcggagagctccccgggttcatcgaggggtgaggtagaggggtgagggaagttctcccagaacacagatgggtactgggagatgttacagggcggcgaacattcgaggagatggagatcaagaccgagaccaggctgatattgtcgacaggagataaccagtagataactactgctactataatggttaggagtatacgtggaatagcaggccgagatgctacggcgaactgcagcaaccgcggacgtggtttgtccgagactaggctggactgggactagcgaccacaaaaccccgccacaaccagtgcctttttttcgcagtccatttgtcgatttcttttcgcctgtgtccccctcttcgtccttccttcgcttttttctcttccttttcctctaatctcctcctcaattccaacatcctcctcacctctccctttcactttttacctcataccccctaatatccatcaccatg

(19) United States
(12) Patent Application Publication
(10) Pub. No.: US 2010/0112638 A1
(43) Pub. Date: May 6, 2010

(54) RECOMBINANT HOST CELL FOR THE PRODUCTION OF A COMPOUND OF INTEREST

(76) Inventors: Cornelis Maria Jacobus Sagt, Utrecht (NL); Noël Nicolaas Maria Elisabeth Van Peij, Delfgauw (NL); Herman Abel Bernard Wosten, Zeist (NL); Alexandra Maria Costa Rodrigues Alves, Haren (NL); Ronald Peter De Vries, Utrecht (NL); Ana Marcela Levin Chucrel, Eliana (ES)

Correspondence Address: NIXON & VANDERHYE, PC, 901 NORTH GLEBE ROAD, 11TH FLOOR, ARLINGTON, VA, 22203, US

(21) Appl. No.: 12/526,289

(22) PCT Filed: Feb. 12, 2008
(86) PCT No.: PCT/EP2008/051680

§ 371 Date: Dec. 30, 2009

(30) Foreign Application Priority Data
Feb. 15, 2007 (EP) 07102493.9

(51) Int. Cl. C12P 21/00; C12N 1/15

(52) U.S. Cl. 435/69.1; 435/254.11; 435/254.3; 435/254.5

(57) ABSTRACT

We describe a recombinant host cell for the production of a compound of interest as well as isolated fungal promoter DNA sequences, to DNA constructs, vectors, and fungal host cells comprising these promoters in operative association with coding sequences encoding a compound of interest. We also describe methods for expressing a gene of interest and/or producing compounds of interest using a promoter according to the invention.

### FIELD OF THE INVENTION

The present invention relates to a recombinant host cell for the production of a compound of interest. The present invention also relates to isolated fungal promoter DNA sequences, to DNA constructs, vectors, and host cells comprising these promoters in operative association with coding sequences encoding a compound of interest. The present invention also relates to methods for expressing a gene of interest and/or producing compounds of interest using a promoter according to the invention.

### BACKGROUND OF THE INVENTION

Production of a recombinant polypeptide in a host cell is usually accomplished by constructing an expression cassette in which the DNA coding for the polypeptide is placed under the expression control of a promoter, suitable for the host cell. The expression cassette may be introduced into the host cell, by plasmid- or vector-mediated transformation. Production of the polypeptide may then be achieved by culturing the transformed host cell under inducing conditions necessary for the proper functioning of the promoter contained in the expression cassette.

For each host cell, expression of a coding sequence which has been introduced into the host by transformation and production of recombinant polypeptides encoded by this coding sequence requires the availability of functional promoters. Numerous promoters are already known to be functional in hosts cells, e.g. from fungal host cells. There are examples of cross-species use of promoters: the promoter of the Aspergillus nidulans (A. nidulans gpdA gene is known to be functional in Aspergillus niger (A. niger) (J. Biotechnol. 1991 January; 17(1):19-33. Intracellular and extracellular production of proteins in Aspergillus under the control of expression signals of the highly expressed A. nidulans gpdA gene. Punt P J, Zegers N D, Busscher M, Pouwels P H, van den Hondel C A.) Another example is the A. niger beta-xylosidase xInD promoter used in A. niger and A. nidulans (Transcriptional regulation of the xylanolytic enzyme system of Aspergillus, van Peij, NNME, PhD-thesis Landbouwuniversiteit Wageningen, the Netherlands, ISBN 90-5808-154-0) and the expression of the Escherichia coli beta-glucuronidase gene in A. niger, A. nidulans and Cladosporium fulvum as described in Curr Genet. 1989 March; 15(3):177-80: Roberts I N, Oliver R P, Punt P J, van den Hondel C A. “Expression of the Escherichia coli beta-glucuronidase gene in industrial and phytopathogenic filamentous fungi”.

However, there is still a need for improvement of the recombinant expression and production of compounds of interest. For example, a known problem to be associated with the production of a compound of interest in a fungal host cell is that only part of the mycelium is involved in the production of the compound. This also applies to other host cells in which expression is not constitutive in all phases of the cell-cycle. It is therefore an objective of the invention to provide improved expression and production systems.

### DESCRIPTION OF THE FIGURES

FIG. 1 depicts the plasmid map of pGBTOPGLA, which is an integrative glucoamylase expression vector.

FIG. 2 depicts the plasmid map of pGBTOPGLA-2, which is an integrative glucoamylase expression vector with a multiple cloning site.

FIG. 3 depicts the plasmid map of pGBTOPGLA-16, which is an integrative expression vector containing a promoter according to the invention in operative association with the glucoamylase coding sequence.

FIG. 4 depicts glucoamylase activity in culture broth for A. niger strains expressing glaA constructs, all under control of distinct promoters. Construct are described in Table 2. Glucoamylase activities are depicted in relative units, with the average of the WT1 cultures at day 4 set at 100%. The two transformants per type indicated were independently isolated and cultivated transformants.

FIG. 5 depicts the plasmid map of pGBDEL-PGLAA, which is a replacement vector.

FIG. 6 depicts a schematic representation of a promoter replacement.

FIG. 7 depicts a schematic representation of integration through homologous recombination.

FIG. 8 depicts spatial activity of laccase in 6 day (upper panel) and 10 day (lower panel) old sandwiched colonies of P. cinnabarinus recombinant strains G13 (A), S1 (B) and L12-8 (C) (see Table 4 for explanation of the strains. Laccase activity is detected as a grey or black staining. Black arrow indicates edge of the colony.

FIG. 9 depicts Laccase activity of strain GS8 (A) and the parental G14 strain (B). Laccase activity is detected as a grey or black staining. In G-S8 the largest part of the mycelium is devoted to secretion.

### DETAILED DESCRIPTION OF THE INVENTION

It is an objective of the invention to provide improved expression systems based on recombinant hosts cells comprising multiple distinct promoter sequences. These promoters may e.g. demonstrate distinct activity during different phases of the cell cycle, or in different parts of a fungal cell or fungal mycelium. They may also be inducible by a specific convenient substrate or compound. Several distinct functional promoters are also advantageous when one envisages to simultaneously overexpress various genes in a single host. To prevent squelching (titration of specific transcription factors), it is preferable to use multiple distinct promoters, e.g. one specific promoter for each gene to be expressed.

Accordingly, the present invention relates to a recombinant host cell comprising at least two DNA constructs, each DNA construct comprising a coding sequence in operative association with a promoter DNA sequence, wherein the at least two DNA constructs comprise at least two distinct promoter DNA sequences and wherein the coding sequences comprised in said DNA constructs encode related polypeptides. Said recombinant host cell will herein be referred to as the recombinant host cell according to the invention. The recombinant host cell according to the invention is advantageously used for the recombinant production of at least one compound of interest. Optionally, said two DNA constructs, are comprised in a single construct. According to an aspect of the invention, the recombinant host comprises at least one DNA construct that is introduced into the host cell by recombinant techniques, i.e. the parental host from which the recombinant host is derived may already contain a native DNA construct comprising a coding sequence encoding a compound of interest.

The compound of interest may for instance be RNA, a polypeptide, a metabolite, or may be the entire host cell or a part thereof. (i.e. biomass or processed biomass, e.g. an extract of biomass).

The term “distinct promoter DNA sequences” is herein defined as promoter DNA sequences that are not both obtained from a single gene. The distinct promoter DNA sequences will be referred to as the promoter sequences according to the invention. According to the invention, a promoter DNA sequence may be native or foreign to the coding sequence and a promoter DNA sequence may be native or foreign to the host cell.

The term, “related polypeptides” is defined herein to encompass polypeptides involved in the production of a single compound of interest. One or more polypeptides may be the actual compound(s) of interest; another polypeptide may optionally be a regulator, e.g. a transcriptional activator of the polypeptide of interest. If the compound of interest is a metabolite, the related polypeptides may be enzymes involved in the production of a metabolite.

Otherwise, the related polypeptides may share a substantial match percentage. According to an embodiment of the invention, the related polypeptides share a match percentage of at least 50%. More preferably, the related polypeptides at least 60%, even more preferably at least 70%, even more preferably at least 80%, even more preferably at least 90%, even more preferably at least 99%. Most preferably, the related polypeptides are identical polypeptides.

For purposes of the present invention, the degree of identity, i.e. the match percentage, between two polypeptides, respectively two nucleic acid sequences is preferably determined using ClustalW as defined in: Thompson J D, Higgins D G, and Gibson T J (1994) ClustalW: improving the sensitivity of progressive multiple sequence alignment through sequence weighting, positions-specific gap penalties and weight matrix choice. Nucleic Acids Research 22:4673-4680.

According to a preferred embodiment, the at least two distinct promoter DNA sequences possess distinct expression characteristics.

According to another preferred embodiment, at least one of the at least two promoter DNA sequences is selected from the group consisting of:
(a) a DNA sequence comprising a nucleotide sequence selected from the set consisting of: SEQ ID NO's:1 to 4 and 13 to 55 and the promoter DNA sequences of the genes listed in Table 1,
(b) a DNA sequence capable of hybridizing with the DNA sequence of (a),
(c) a DNA sequence sharing at least 80% homology with the DNA sequence of (a),
(d) a variant of any of the DNA sequences of (a) to (c), and
(e) a subsequence of any of the DNA sequences of (a) to (d),
   wherein preferably at least two distinct promoter DNA sequences possess distinct expression characteristics.

The promoter DNA sequences of the genes depicted in Table 1 are for the purpose according to the invention defined as the 1500 bp nucleotide sequence immediately upstream of the startcodon (ATG) of the respective gene. According to a more preferred embodiment of the invention, at least one of the at least two distinct promoter DNA sequences is selected from the promoter DNA sequences of the genes depicted in Table 1, wherein preferably at least two distinct promoter DNA sequences possess distinct expression characteristics, i.e. are selected from different columns (e.g. a promoter DNA sequence of a gene listed in column I combined with a promoter DNA sequence of gene listed in column II of Table 1).

Preferred promoter combinations are: the promoter DNA sequence of An03g06550 (e.g. glaA promoter with optimized translation initiation site as provided in SEQ ID NO: 16) combined with at least one of the promoter DNA sequences of An12g06930 or An05g02100 (e.g. amyB promoters as mentioned in WO 2006/092396 with optimized translation initiation site (as detailed in WO 2006/077258); SEQ ID NO: 17 represents promoter DNA sequence An12g06930 with optimized translation initiation site).

Other preferred promoter combinations are: at least one of the promoter DNA sequences of the set of genes An03g06550 (e.g. glaA promoter with optimized translation initiation site as provided in SEQ ID NO: 16), An12g06930 and/or An05g02100 (e.g. amyB promoters as mentioned in WO 2006/092396; SEQ ID NO: 17 represents promoter DNA sequence An12g06930 with optimized translation initiation site) combined with at least one of the promoter DNA sequences of An16g01830 (gpdA promoter as provided in SEQ ID NO: 18), promoters as mentioned in WO 2005/100573 (e.g. promoters as provided in SEQ ID NO: 13, 14, 15, 19 or 20).

Other preferred promoter combinations are: at least one of the promoter DNA sequences of the set of genes An03g06550 (e.g. glaA promoter with optimized translation initiation site as provided in SEQ ID NO: 16), An12g06930 and/or An05g02100 (e.g. amyB promoters as mentioned in WO 2006/092396; SEQ ID NO: 17 represents promoter DNA sequence An12g06930 with optimized translation initiation site), An16g01830 (gpdA promoter as provided in SEQ ID NO: 18), promoters as mentioned in WO 2005/100573 (e.g. promoters as provided in SEQ ID NO: 13, 14, 15, 19 or 20), combined with at least one of the promoter DNA sequences of the genes listed in column II of Table 1.

TABLE 1
Aspergillus niger gene sequences. Columns I, II and III represent
differentially expressed genes, i.e. disctinct expression
characteristics of the promoter of the gene, wherein “distinct
expression characteristics” are as defined below.

| I | II | III |
|---|---|---|
| An03g06550 | An15g01590 | An15g07090 |
| An15g07700 | An14g00420 | An04g06510 |
| An11g01630 | An01g03090 | An14g03080 |
| An08g05640 | An02g07020 | An11g10490 |
| An02g10320 | An04g08150 | An08g03490 |
| An01g06860 | An16g09070 | An18g05640 |
| An04g08190 | An07g00070 | An01g03480 |
| An04g06380 | An04g09490 | An08g01960 |
| An15g03940 | An18g03380 | An09g06790 |
| An04g03360 | An15g01580 | An07g03880 |
| An04g00990 | An08g09880 | An14g00010 |
| An11g03340 | An03g02400 | An08g10060 |
| An04g06920 | An03g02360 | An01g05960 |
| An16g03330 | An01g11660 | An12g04870 |
| An16g07110 | An01g01950 | An04g02260 |
| An11g01660 | An17g00230 | An06g01550 |
| An01g07140 | An08g08370 | An08g06960 |
| An16g02930 | An18g05480 | An15g01700 |
| An12g00710 | An02g14590 | An01g02900 |
| An16g05930 | An12g09270 | An16g05920 |
| An16g05920 | An16g01880 | An08g07290 |
| An13g00320 | An11g02540 | An07g09990 |
| An12g06930 | An02g00090 | An17g00550 |
| An05g02100 | An09g00840 | An16g01830 |
| | An01g10930 | An18g04840 |
| | | An18g04220 |

The gene numbers refer to the sequences published by Pel et al., Genome sequencing and analysis of the versatile cell factory Aspergillus niger CBS 513.88, Nature Biotechnology 25, 221-231 (2007), said sequences are herein incorporated by reference. The annotated genome of Aspergillus niger CBS513.88 has been deposited at the EMBL database.

The term “host cell” encompasses all suitable eukaryotic and prokaryotic host cells. The choice of a host cell will to a large extent depend upon the gene encoding the compound of interest and its source. The skilled person knows how to select appropriate host cell.

According to one embodiment, the recombinant host cell according to the invention is used for the production of a polypeptide.

According to another embodiment, the recombinant host cell according to the invention is used for the production of specific primary or secondary metabolites, said metabolites being the compound of interest, such as (beta-lactam) antibiotics, vitamins or carotenoids.

The term “DNA construct” is defined herein as a nucleic acid molecule, either single or double-stranded, which is isolated from a naturally occurring gene or which has been modified to contain segments of nucleic acid combined and juxtaposed in a manner that would not otherwise exist in nature.

The term “coding sequence” is defined herein as a nucleic acid sequence that is transcribed into mRNA, which is translated into a polypeptide when placed under the control of the appropriate control sequences. The boundaries of the coding sequence are generally determined by the ATG start codon, which is normally the start of the open reading frame at the 5′ end of the mRNA and a transcription terminator sequence located just downstream of the open reading frame at the 3′ end of the mRNA. A coding sequence can include, but is not limited to, genomic DNA, cDNA, semisynthetic, synthetic, and recombinant nucleic acid sequences.

In the context of this invention, a promoter DNA sequence is a DNA sequence, which is capable of controlling the expression of a coding sequence, when this promoter DNA sequence is in operative association with this coding sequence. A promoter DNA sequence can include, but is not limited to, genomic DNA, semisynthetic, synthetic, and recombinant nucleic acid sequences.

The term “promoter” or “promoter sequence”, which terms are used interchangeably, is defined herein as a DNA sequence that binds the RNA polymerase and directs the polymerase to the correct transcriptional start site of a coding sequence to initiate transcription. RNA polymerase effectively catalyzes the assembly of messenger RNA complementary to the appropriate DNA strand of the coding region. The term “promoter” or “promoter sequence” will also be understood to include the 5′ non-coding region (between promoter and translation start) for translation after transcription into mRNA, cis-acting transcription control elements such as enhancers, and other nucleotide sequences capable of interacting with transcription factors.

The term “in operative association” is defined herein as a configuration in which a promoter DNA sequence is appropriately placed at a position relative to a coding sequence such that the promoter DNA sequence directs the production of a polypeptide encoded by the coding sequence.

The term “distinct expression characteristics” is herein defined as a difference in expression level between at least two promoters when assayed under similar conditions, which difference occurs in at least one occasion. Examples of such occasions are listed below, but are depicted for illustrative purposes and are not to be construed as an exhaustive listing.

Distinct expression characteristics comprise expression differences which are the result of cellular differentiation. Cellular differentiation is a concept from developmental biology describing the process by which cells acquire a “type”. The genetic material of a cell or organism remains the same, with few exceptions, but for example morphology may change dramatically during differentiation. Differentiation can involve changes in numerous aspects of cell physiology; size, shape, polarity, metabolic activity, responsiveness to signals or gene expression profiles can all change during differentiation. As such, a fungus can be differentiated between different parts of a hypha (such as hyphal tip versus the subapical part of the hypha) or between different parts of the mycelium (such as between the centre and the periphery of a vegetative mycelium, or between the vegetative mycelium and a reproductive structure such as a fruiting body, or a conidiophore).

Differentiation can occur for example as a result of age, growth condition, nutrient composition, stress (environmental condition), temperature, radiation, pressure, morphology, light, growth speed, fermentation type (batch, fed-batch, continuous growth condition, submerged, surface or solid state). An example of nutrient composition mediated differentiation is the induction of the transcription factor XInR in Aspergillus by the nutrient xylose. The transcription factor XInR induces the transcription of various genes encoding extracellular enzymes, whereas transcription of other genes is not induced (de Vries and Visser, Microb. Mol. Biol. Rev. 65: 497-522).

Differentiation, which may involve changes in numerous aspects of cell physiology, may find its basis at the transcription, translation, protein expression or enzyme activity level. The differentiation may start at the DNA, the RNA, mRNA, protein or enzyme activity or metabolite levels. Differentiation and thus distinct expression characteristics can be identified by measuring and comparing the DNA, the RNA, mRNA, protein or enzyme activity or metabolite levels between (potentially) differentiated cells. The (metabolic, protein of RNA) differences identified can be related to the genes involved. These genes are candidate genes possessing distinct expression characteristics. The expression profile and possible distinct expression characteristic can be investigated by measuring the mRNA levels of the genes between (potentially) differentiated cells under investigation. Differentially expressed genes contain a promoter with distinct expression characteristics. Differentially expressed genes differ preferably at least 2-fold in expression level, more preferably at least 3-fold, even more preferably at least 5-fold, even more preferably at least 10-fold, even more preferably at least 20-fold, even more preferably at least 50-fold and most preferably at least 100-fold. Preferably, one promoter results in undetectable expression compared to another promoter resulting in high level expression when assayed under similar conditions.

For application of promoter DNA sequences possessing distinct expression characteristics, the distinct expression characteristics may relate to industrial relevant conditions and processes. These conditions of differentiation relate to but are not limited to spatial, temporal, environmental or nutritional differences between cells occurring during industrial growth and production of biomass and product.

Distinct expression characteristics may be reflected during different phases of the cell cycle, or in different parts of the cell.

### Recombinant Host Cells

According to the invention, the recombinant host cell according to the invention may be any suitable eukaryotic and prokaryotic host cell.

According to an embodiment, the recombinant host cell is a prokaryotic cell. The prokaryotic host cell may be any prokaryotic host cell useful in the methods of the present invention. Preferably, the prokaryotic host cell is bacterial cell. The term “bacterial cell” includes both Gram-negative and Gram-positive microorganisms. Suitable bacteria may be selected from e.g. Escherichia, Anabaena, Caulobacter, Gluconobacter, Rhodobacter, Pseudomonas, Para coccus, Bacillus, Brevibacterium, Corynebacterium, Rhizobium (Sinorhizobium), Flavobacterium, Klebsiella, Enterobacter, Lactobacillus, Lactococcus, Methylobacterium, Propionibacterium, Staphylococcus or Streptomyces. Preferably, the bacterial cell is selected from the group consisting of B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Para coccus denitrificans, E. coli, C. glutamicum, Staphylococcus carnosus, Streptomyces lividans, Sinorhizobium melioti and Rhizobium radiobacter.

According to an embodiment, the recombinant host cell is a eukaryotic cell. The eukaryotic host cell may be any eukaryotic host cell useful in the methods of the present invention. Preferably, the eukaryotic cell is a mammalian, insect, plant, fungal, or algal cell. Preferred mammalian cells include e.g. Chinese hamster ovary (CHO) cells, COS cells, 293 cells, PerC6 cells, and hybridomas. Preferred insect cells include e.g. Sf9 and Sf21 cells and derivatives thereof. More preferably, the recombinant host cell is a fungal host cell. The fungal host cell may be any fungal cell useful in the methods of the present invention. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., supra) and all mitosporic fungi (Hawksworth et al., supra).

According to preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

According to a more preferred embodiment, the yeast host cell is a Candida, Hansenula, Kluyveromyces, Pichia, Saccharomyces, Schizosaccharomyces, or Yarrowia cell.

According to an even more preferred embodiment, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. According to another even more preferred embodiment, the yeast host cell is a Kluyveromyces lactis cell. According to another even more preferred embodiment, the yeast host cell is a Yarrowia lipolytica cell.

According to another preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is mostly obligatory aerobic. In contrast, vegetative growth by yeasts such as Saccharomyces cerevisiae is by budding of a unicellular thallus and carbon catabolism may be fermentative.

According to a more preferred embodiment, the filamentous fungal host cell is a cell of a species of, but not limited to, Acremonium, Agraricus, Aspergillus, Aureobasidium, Chrysosporum, Coprinus, Cryptococcus, Filibasidium, Flammulina, Fusarium, Humicola, Magnaporthe, Mucor, Myceliophthora, Neocallimastix, Neurospora, Paecilomyces, Penicillium, Phanerochaete, Piromyces, Pleurotus, Schizophyllum, Shiitake, Talaromyces, Thermoascus, Thielavia, Tolypocladium, Trametes, or Trichoderma strain.

According to an even more preferred embodiment, the filamentous fungal host cell is an Aspergillus awamori, Aspergillus aculeatus, Aspergillus foetidus, Aspergillus japonicus, A. nidulans, Asprgillus niger, Aspergillus sojae, Aspergillus tubigenis, Aspergillus vadensis or Aspergillus oryzae cell. According to another even more preferred embodiment, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatun, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. According to another even more preferred embodiment, the filamentous fungal host cell is a Agraricus bisprorus, Chrysosporium lucknowense, Humicola insolens, Humicola lanuginosa, Mucor miehei, Myceliophthora thermophila, Neurospora crassa, Penicillium purpurogenum, Penicillium chrysogenum, Pycnoporus cinnabarinus, Thielavia terrestris, Trichoderma harzianum, Trichoderma koningii, Trichoderma longibrachiatum, Trichoderma reesei, or Trichoderma viride cell.

Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), Banque de Resources Fongiques de Marseille, France, and Agricultural Research Service Patent Culture Collection, Northern Regional Research Center (NRRL) Aspergillus niger CBS 513.88, Aspergillus oryzae ATCC 20423, IFO 4177, ATCC 1011, ATCC 9576, ATCC14488-14491, ATCC 11601, ATCC12892, P. chrysogenum CBS 455.95, Penicillium citrinum ATCC 38065, Penicillium chrysogenum P2, Acremonium chrysogenum ATCC 36225 or ATCC 48272, Trichoderma reesei ATCC 26921 or ATCC 56765 or ATCC 26921, Aspergillus sojae ATCC11906, Chrysosporium lucknowense ATCC44006, Pycnoporus cinnabarinus BRFM44, and derivatives thereof.

Optionally, the filamentous fungal host cell comprises an elevated unfolded protein response (UPR) compared to the wild type cell to enhance production abilities of a compound of interest. UPR may be increased by techniques described in US2004/0186070A1 and/or US2001/0034045A1 and/or W001/72783A2. More specifically, the protein level of HAC1 and/or IRE1 and/or PTC2 has been modulated in order to obtain a host cell having an elevated UPR.

Alternatively, or in combination with an elevated UPR, the filamentous fungal host cell may comprise a specific one-way mutation of the sec61 translocation channel between ER and cytoplasm as described in WO2005/123763. Such mutation confers a phenotype wherein de novo synthesised polypeptides can enter the ER through sec61, however, retrograde transport through sec61 is impaired in this one-way mutant.

Alternatively, or in combination with an elevated UPR and/or one-way mutation of the sec61 translocation channel, the filamentous fungal host cell is genetically modified to obtain a phenotype displaying lower protease expression and/or protease secretion compared to the wild type cell in order to enhance production abilities of a compound of interest. Such phenotype may be obtained by deletion and/or modification and/or inactivation of a transcriptional regulator of expression of proteases. Such a transcriptional regulator is e.g. prtT. Lowering expression of proteases by modulation of prtT is preferable performed by techniques described in US2004/0191864A1, WO2006/04312 and WO2007/062936.

Alternatively, or in combination with an elevated UPR and/or and/or one-way mutation of the sec61 translocation channel, a phenotype displaying lower protease expression and/or protease secretion, the filamentous fungal host cell displays an oxalate deficient phenotype in order to enhance the yield of production of a compound of interest. An oxalate deficient phenotype is preferable obtained by techniques described in WO2004/070022, which is herein enclosed by reference.

Alternatively, or in combination with an elevated UPR and/or and/or one-way mutation of the sec61 translocation channel, a phenotype displaying lower protease expression and/or protease secretion and/or oxalate deficiency, the filamentous fungal host cell displays a combination of phenotypic differences compared to the wild type cell to enhance the yield of production of the compound of interest. These differences may include, but are not limited to, lowered expression of glucoamylase and/or neutral alpha-amylase A and/or neutral alpha-amylase B, protease, and oxalic acid hydrolase. Said phenotypic differences displayed by the filamentous fungal host cell may be obtained by genetic modification according to the techniques described in US2004/0191864A1.

### Promoter DNA Sequences

Promoter activity is preferably determined by measuring the concentration of the protein(s) coded by the coding sequence(s), which is (are) in operative association with the promoter. Alternatively the promoter activity is determined by measuring the enzymatic activity of the protein(s) coded by the coding sequence(s), which is (are) in operative association with the promoter. Most preferably, the promoter activity (and its strength) is determined by measuring the expression of the coding sequence of the IacZ reporter gene (Luo (Gene 163 (1995) 127-131). According to another preferred method, the promoter activity is determined by using the green fluorescent protein as coding sequence (Microbiology. 1999 March; 145 (Pt 3):729-34. Santerre Henriksen A L, Even S, Muller C, Punt P J, van den Hondel C A, Nielsen J. Study)

Additionally, promoter activity can be determined by measuring the mRNA levels of the transcript generated under control of the promoter. The mRNA levels can, for example, be measured by Northern blot or real time quantitative PCR (J. Sambrook, 2000, Molecular Cloning, A Laboratory Manual, 3d edition, Cold Spring Harbor, N.Y.).

According to an aspect of the invention, the promoter DNA sequence according to the invention is a DNA sequence capable of hybridizing with a DNA sequence comprising a nucleotide sequence selected from the set consisting of: SEQ ID NO's:1 to 4 and 13 to 55 and the promoter DNA sequences of the genes listed in Table 1. According to a preferred embodiment, the promoter DNA sequence according to the invention is a DNA sequence comprising a nucleotide sequence selected from the set consisting of: SEQ ID NO's:1 to 4 and 13 to 55 and the promoter DNA sequences of the genes listed in Table 1. The present invention encompasses (isolated) promoter DNA sequences that retain promoter activity and hybridize under very low stringency conditions, preferably low stringency conditions, more preferably medium stringency conditions, more preferably medium-high stringency conditions, even more preferably high stringency conditions, and most preferably very high stringency conditions with a nucleic acid probe that corresponds to:
(i) nucleotides 1 to 1500 of: a sequence selected from the set of SEQ ID NO's:1 to 4 and 13 to 55 and the promoter DNA sequences of the genes listed in Table 1, preferably nucleotides 100 to 1490, more preferably 200 to 1480, even more preferably 300 to 1470, even more preferably 350 to 1450 and most preferably 360 to 1400
(ii) is a subsequence of (i), or
(iii) is a complementary strand of (i), (ii), (J. Sambrook et al., supra).

The subsequence of a sequence selected from the set of SEQ ID NO's:1 to 4 and 13 to 55 and the promoter DNA sequences of the genes listed in Table 1, may be at least 100 nucleotides, preferably at least 200 nucleotides, more preferably at least 300 nucleotides, even more preferably at least 400 nucleotides and most preferably at least 500 nucleotides. Hybridization conditions are as defined further in the description.

The nucleic acid sequence of a sequence selected from the set of SEQ ID NO's:1 to 4 and 13 to 55 and the promoter DNA sequences of the genes listed in Table 1, or a subsequence thereof may be used to design a nucleic acid probe to identify and clone DNA promoters from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 15, preferably at least 25, and more preferably at least 35 nucleotides in length. Additionally, such probes can be used to amplify DNA promoters by PCR. Longer probes can also be used. DNA, RNA and Peptide Nucleid Acid (PNA) probes can be used. The probes are typically labelled for detecting the corresponding gene (for example, with 32P, 33P 3H, 35S, biotin, or avidin or a fluorescent marker). Such probes are encompassed by the present invention.

Thus, a genomic DNA or cDNA library prepared from such other organisms may be screened for DNA, which hybridizes with the probes described above and which encodes a polypeptide. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that shares homology with a sequence selected from the set of SEQ ID NO's:1 to 4 and 13 to 55 and the promoter DNA sequences of the genes listed in Table 1, or a subsequence thereof, the carrier material may be used in a Southern blot.

For purposes of the present invention, hybridization indicates that the nucleic acid sequence hybridizes to a labeled nucleic acid probe corresponding to a nucleic acid sequence selected from the set of SEQ ID NO's:1 to 4 and 13 to 55, the complementary strand, or subsequence thereof or corresponding to a promoter DNA sequence of one of the genes listed in Table 1, the complementary strand, or subsequence thereof, under very low to very high stringency conditions. Molecules to which the nucleic acid probe hybridizes under these conditions are detected using for example a X-ray film. Other hybridisation techniques also can be used, such as techniques using fluorescence for detection and glass sides and/or DNA microarrays as support. An example of DNA microarray hybridisation detection is given in FEMS Yeast Res. 2003 December; 4(3):259-69 (Daran-Lapujade P, Daran J M, Kotter P, Petit T, Piper M D, Pronk J T. “Comparative genotyping of the Saccharomyces cerevisiae laboratory strains S288C and CEN.PK113-7D using oligonucleotide microarrays”. Additionally, the use of PNA microarrays for hybridization is described in Nucleic Acids Res. 2003 Oct. 1; 31(19): 119 (Brandt O, Feldner J, Stephan A, Schroder M, Schnolzer M, Arlinghaus H F, Hoheisel J D, Jacob A. PNA microarrays for hybridisation of unlabelled DNA samples.)

Preferably, the nucleic acid probe is a nucleic acid sequence selected from the set of SEQ ID NO's:1 to 4 and 13 to 55 or of a promoter DNA sequence of one of the genes listed in Table 1. More preferably, the nucleic acid probe is the sequence having nucleotides 20 to 1480 of a sequence selected from the set of SEQ ID NO's:1 to 4 and 13 to 55 or of a promoter DNA sequence of one of the genes listed in Table 1, more preferably nucleotides 500 to 1450, even more preferably nucleotides 800 to 1420, and most preferably nucleotides 900 to 1400 of a sequence selected from the set of SEQ ID NO's:1 to 4 and 13 to 55 or of a promoter DNA sequence of one of the genes listed in Table 1. Another preferred probe is the part of the DNA sequence upstream of the transcription start site.

For long probes of at least 100 nucleotides in length, very low to very high stringency conditions are defined as prehybridization and hybridization at 42 DEG C. in 5×SSPE, 0.3% SDS, 200 microgram/ml sheared and denatured salmon sperm DNA, and either 25% formamide for very low and low stringencies, 35% formamide for medium and medium-high stringencies, or 50% formamide for high and very high stringencies, following standard Southern blotting procedures.

For long probes of at least 100 nucleotides in length, the carrier material is finally washed three times each for 15 minutes using 2×SSC, 0.2% SDS preferably at least at 45 DEG C. for very low stringency, more preferably at least at 50 DEG C. for low stringency, more preferably at least at 55 DEG C. for medium stringency, more preferably at least at 60 DEG C. for medium-high stringency, even more preferably at least at 65 DEG C. for high stringency, and most preferably at least at 70 DEG C. for very high stringency.

For short probes which are about 15 nucleotides to about 100 nucleotides in length, stringency conditions are defined as prehybridization, hybridization, and washing post-hybridization at 5 DEG C. to 10 DEG C. below the calculated Tm using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to about 100 nucleotides in length, the carrier material is washed once in 6×SCC plus 0.1% SDS for 15 minutes and twice each for 15 minutes using 6×SSC at 5 DEG C. to 10 DEG C. below the calculated Tm.

According to another embodiment, a sequence selected from the set of SEQ ID NO's:1 to 4 and 13 to 55 or a promoter DNA sequence of one of the genes listed in Table 1 is first used to clone the native gene, coding sequence of said native gene or part of it, which is operatively associated with a sequence selected from the set of SEQ ID NO's:1 to 4 and 13 to 55 or a promoter DNA sequence of one of the genes listed in Table 1. This can be done starting with either a sequence selected from the set of SEQ ID NO's:1 to 4 and 13 to 55, a promoter DNA sequence of one of the genes listed in Table 1, or a subsequence thereof as earlier defined and using this sequence as a probe. The probe is hybridised to a cDNA or a genomic library of a given host, either Aspergillus niger or any other fungal host as defined in this application. Once the native gene or part of it is cloned, it can be subsequently used itself as a probe to clone genes that share homology to the native gene derived from other fungi by hybridisation experiments as described herein. Preferably, the gene shares at least 55% homology with the native gene, more preferably at least 60%, more preferably at least 65%, more preferably at least 70%, even more preferably at least 75% preferably about 80%, more preferably about 90%, even more preferably about 95%, and most preferably about 97% homology with the native gene. The sequence upstream of the coding sequence of the gene sharing homology with the native gene is a promoter encompassed by the present invention.

Alternatively, the sequence of the native gene, coding sequence or part of it, which is operatively associated with a promoter according to the invention can be identified by using a sequence selected from the set of SEQ ID NO's:1 to 4 and 13 to 55, or a subsequence thereof as earlier defined, or a gene sequence listed in Table 1, or a subsequence thereof, to search genomic databases using for example an alignment or BLAST algorithm as described herein. This resulting sequence can subsequently be used to identify orthologues or homologous genes in any other fungal host as defined in this application. The sequence upstream the coding sequence of the identified orthologue or homologous gene is a promoter encompassed by the present invention.

According to yet another embodiment, the promoter DNA sequence according to the invention is a(n) (isolated) DNA sequence, which shared at least 80% homology (identity) to a sequence selected from the set of SEQ ID NO's:1 to 4 and 13 to 55 or to a promoter DNA sequence of one of the genes listed in Table 1. Preferably, the DNA sequence shares at least preferably about 85%, more preferably about 90%, even more preferably about 95%, and most preferably about 97% homology with a sequence selected from the set of SEQ ID NO's:1 to 4 and 13 to 55 or with a promoter DNA sequence of one of the genes listed in Table 1.

For purposes of the invention, the terms “homology” and “identity” are used interchangeably.

The degree of homology (identity) between two nucleic acid sequences is preferably determined by the BLAST program. Software for performing BLAST analyses is publicly available through the National Center for Biotechnology Information (http://www.ncbi.nlm.nih.gov/). The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89: 10915 (1989)) alignments (B) of 50, expectation (E) of 10, M=5, N=−4, and a comparison of both strands.

According to yet another embodiment of the invention, the promoter DNA sequence is a variant of a sequence selected from the set of SEQ ID NO's:1 to 4 and 13 to 55 or of a promoter DNA sequence of one of the genes listed in Table 1.

The term “variant” or “variant promoter” is defined herein as a promoter having a nucleotide sequence comprising a substitution, deletion, and/or insertion of one or more nucleotides of a parent promoter, wherein the variant promoter has more or less promoter activity than the corresponding parent promoter. The term “variant promoter” will encompass natural variants and in vitro generated variants obtained using methods well known in the art such as classical mutagenesis, site-directed mutagenesis, and DNA shuffling. A variant promoter may have one or more mutations. Each mutation is an independent substitution, deletion, and/or insertion of a nucleotide.

According to a preferred embodiment, the variant promoter is a promoter, which has at least a modified regulatory site as compared to the promoter sequence first identified (e.g. a sequence selected from the set of SEQ ID NO's:1 to 4 and 13 to 55 or a promoter DNA sequence of one of the genes listed in Table 1). Such a regulatory site can be removed in its entirety or specifically mutated as explained above. The regulation of such promoter variant is thus modified so that for example it is no longer induced by glucose. Examples of such promoter variants and techniques on how to obtain these are described in EP 673 429 or in WO 94/04673.

The promoter variant can be an allelic variant. An allelic variant denotes any of two or more alternative forms of a gene occupying the same chromosomal locus. Allelic variation arises naturally through mutation, and may result in polymorphism within populations. The variant promoter may be obtained by (a) hybridizing a DNA under very low, low, medium, medium-high, high, or very high stringency conditions with (i) a sequence selected from the set of SEQ ID NO's:1 to 4 and 13 to 55 or a promoter DNA sequence of one of the genes listed in Table 1, (ii) a subsequence of (i) or (iii) a complementary strand of (i), (ii), and (b) isolating the variant promoter from the DNA. Stringency and wash conditions are as defined herein.

According to yet another embodiment, the promoter is a subsequence of a sequence selected from the set of SEQ ID NO's:1 to 4 and 13 to 55 or of a promoter DNA sequence of one of the genes listed in Table 1, said subsequence still having promoter activity. The subsequence preferably contains at least about 100 nucleotides, more preferably at least about 200 nucleotides, and most preferably at least about 300 nucleotides.

According to another preferred embodiment, a subsequence is a nucleic acid sequence encompassed by a sequence selected from the set of SEQ ID NO's:1 to 4 and 13 to 55 or by a promoter DNA sequence of one of the genes listed in Table 1, wherein one or more nucleotides from the 5′ and/or 3′ end are deleted, said nucleic acid sequence still having promoter activity.

According to another preferred embodiment, the promoter subsequence is a ‘trimmed’ promoter sequence, i.e. a sequence fragment which is upstream from translation start and/or from transcription start. An example of trimming a promoter and functionally analysing it is described in Gene. 1994 Aug. 5; 145(2):179-87: the effect of multiple copies of the upstream region on expression of the Aspergillus niger glucoamylase-encoding gene. Verdoes J C, Punt P J, Stouthamer A H, van den Hondel C A).

The promoter according to the invention can be a promoter, whose sequence may be provided with linkers for the purpose of introducing specific restriction sites facilitating ligation of the promoter sequence with the coding region of the nucleic acid sequence encoding a polypeptide.

Unless otherwise indicated, all nucleotide sequences determined by sequencing a DNA molecule herein were determined using an automated DNA sequencer. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art.

The person skilled in the art is capable of identifying such erroneously identified bases and knows how to correct for such errors.

The sequence information as provided herein should therefore not be so narrowly construed as to require inclusion of erroneously identified bases. The specific sequences disclosed herein can readily be used to isolate the original DNA sequence e.g. from a filamentous fungus, in particular Aspergillus niger, and be subjected to further sequence analyses thereby identifying sequencing errors.

The present invention encompasses functional promoter equivalents typically containing mutations that do not alter the biological function of the promoter it concerns. The term “functional equivalents” also encompasses orthologues of the A. niger DNA sequences. Orthologues of the A. niger DNA sequences are DNA sequences that can be isolated from other strains or species and possess a similar or identical biological activity.

The promoter sequences of the present invention may be obtained from microorganisms of any genus. For purposes of the present invention, the term “obtained from” as used herein in connection with a given source shall mean that the polypeptide is produced by the source or by a cell in which a gene from the source has been inserted.

According to an embodiment of the invention, the promoter sequences are obtained from a prokaryotic source, preferably from a species of Escherichia, Anabaena, Caulobactert, Gluconobacter, Rhodobacter, Pseudomonas, Para coccus, Bacillus, Brevibacterium, Corynebacterium, Rhizobium (Sinorhizobium), Flavobacterium, Klebsiella, Enterobacter, Lactobacillus, Lactococcus, Methylobacterium, Propionibacterium, Staphylococcus or Streptomyces. More preferably, promoter sequences are obtained from B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Paracoccus denitrificans, E. coli, C. glutamicum, Staphylococcus carnosus, Streptomyces lividans, Sinorhizobium melioti or Rhizobium radiobacter.

According to another embodiment, the promoter sequences are obtained from a fungal source, preferably from a yeast strain such as a Candida, Hansenula, Kluyveromyces, Pichia, Saccharomyces, Schizosaccharomyces, or Yarrowia strain, more preferably.from a Saccharomyces carisbergensis, Saccharomyces cerevisiae, Saccharomyces diastaticus, Saccharomyces douglasii, Saccharomyces kluyveri, Saccharomyces norbensis or Saccharomyces oviformis strain. According to another more preferred embodiment, the promoter sequences are obtained from a Kluyveromyces lactis strain. According to another more preferred embodiment, the promoter sequences are obtained from a Yarrowia lipolytica strain.

According to yet another embodiment, the promoter sequences are obtained from a filamentous fungal strain such as an Acremonium, Agraricus, Aspergillus, Aureobasidium, Chrysosporum, Coprinus, Cryptococcus, Filibasidium, Fusarium, Humicola, Magnaporthe, Mucor, Myceliophthora, Neocallimastix, Neurospora, Paecilomyces, Penicillium, Piromyces, Panerochaete, Pleurotus, Schizophyllum, Talaromyces, Thermoascus, Thielavia, Tolypocladium, or Trichoderma strain, more preferably from an Agraricus bisporus, Aspergillus aculeatus, Aspergillus awamori, Aspergillus foetidus, Aspergillus japonicus, Aspergillus nidulans, Aspergillus niger, Aspergillus sojae, Aspergillus tubigenis, Aspergillus oryzae, Aspergillus vadensis, Chrysosporum lucknowense, Humicola insolens, Humicola lanuginosa, Mucor miehei, Myceliophthora thermophila, Neurospora crassa, Penicillium purpurogenum, Penicillium chrysogenum, Pycnoporus cinnabarinus, Schizophyllum commune, Thielavia terrestris, Trichoderma harzianum, Trichoderma koningii, Trichoderma longibrachiatum, Trichoderma reesei, or Trichoderma viride strain.

According to yet another preferred embodiment, the promoter sequences are obtained from a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum strain.

It will be understood that for the aforementioned species, the invention encompasses the perfect and imperfect states, and other taxonomic equivalents, e.g., anamorphs, regardless of the species name by which they are known. Those skilled in the art will readily recognize the identity of appropriate equivalents. Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

Furthermore, promoter sequences according to the invention may be identified and obtained from other sources including microorganisms isolated from nature (e.g, soil, composts, water, etc.) using the above-mentioned probes. Techniques for isolating microorganisms from natural habitats are well known in the art. The nucleic acid sequence may then be derived by similarly screening a genomic DNA library of another microorganism. Once a nucleic acid sequence encoding a promoter has been detected with the probe(s), the sequence may be isolated or cloned by utilizing techniques which are known to those of ordinary skill in the art (see, e.g., Sambrook et al., supra).

In the present invention, the promoter DNA sequence may also be a hybrid promoter comprising a portion of one or more promoters of the present invention; a portion of a promoter of the present invention and a portion of another known promoter, e.g., a leader sequence of one promoter and the transcription start site from the other promoter; or a portion of one or more promoters of the present invention and a portion of one or more other promoters. The other promoter may be any promoter sequence, which shows transcriptional activity in the fungal host cell of choice including a variant, truncated, and hybrid promoter, and may be obtained from genes encoding extracellular or intracellular polypeptides either homologous or heterologous to the host cell. The other promoter sequence may be native or foreign to the nucleic acid sequence encoding the polypeptide and native or foreign to the cell.

According to preferred a embodiment, important regulatory subsequences of the promoter identified can be fused to other ‘basic’ promoters to enhance their promoter activity (as for example described in Mol. Microbiol. 1994 May; 12(3):479-90. Regulation of the xylanase-encoding xlnA gene of Aspergillus tubigensis. de Graaff L H, van den Broeck H C, van Ooyen A J, Visser J.).

Other examples of other promoters useful in the construction of hybrid promoters with the promoters of the present invention include the promoters obtained from the genes for A. oryzae TAKA amylase, Rhizomucor miehei aspartic proteinase, A. niger neutral alpha-amylase, A. niger acid stable alpha-amylase, A. niger or Aspergillus awamori glucoamylase (glaA), A. niger gpdA, A. niger glucose oxidase goxC, Rhizomucor miehei lipase, A. oryzae alkaline protease, A. oryzae triose phosphate isomerase, A. nidulans acetamidase, and Fusarium oxysporum trypsin-like protease (WO 96/00787), as well as the NA2-tpi promoter (a hybrid of the promoters from the genes for A. niger neutral alpha-amylase and A. oryzae triose phosphate isomerase), Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase, and mutant, truncated, and hybrid promoters thereof. Other useful promoters for yeast host cells are described by Romanoset al., 1992, Yeast 8: 423-488.

In the present invention, the promoter DNA sequence may or may not be a “tandem promoter”. A “tandem promoter” is defined herein as two or more promoter sequences each of which is in operative association with a coding sequence and mediates the transcription of the coding sequence into mRNA.

The tandem promoter comprises two or more promoters of the present invention or alternatively one or more promoters of the present invention and one or more other known promoters, such as those exemplified above useful for the construction of hybrid promoters. The two or more promoter sequences of the tandem promoter may simultaneously promote the transcription of the nucleic acid sequence. Alternatively, one or more of the promoter sequences of the tandem promoter may promote the transcription of the nucleic acid sequence at different stages of growth of the cell or morphological different parts of the mycelia.

In the present invention, the promoter may be foreign to the coding sequence encoding a compound of interest and/or to the fungal host cell. A variant, hybrid, or tandem promoter of the present invention will be understood to be foreign to a coding sequence encoding a compound of interest, even if the wild-type promoter is native to the coding sequence or to the fungal host cell.

A variant, hybrid, or tandem promoter of the present invention has at least about 20%, preferably at least about 40%, more preferably at least about 60%, more preferably at least about 80%, more preferably at least about 90%, more preferably at least about 100%, even more preferably at least about 200%, most preferably at least about 300%, and most preferably at least about 400% of the promoter activity of a parental promoter, where the variant, hybrid or tandem promoter originates from.

### Coding Sequences

In the present invention, the coding sequence in the DNA construct according to the invention may encode a polypeptide. The polypeptide may be any polypeptide having a biological activity of interest. The polypeptide may be homologous or heterologous to the host cell according to the invention. Preferably, the polypeptide is an enzyme.

The term “polypeptide” is not meant herein to refer to a specific length of the encoded product and, therefore, encompasses peptides, oligopeptides, and proteins.

The term “homologous gene” or “homologous polypeptide” is herein defined as a gene or polypeptide that is obtainable from a strain that belongs to the same species, including variants thereof, as does the strain actually containing the gene or polypeptide. Preferably, the donor and acceptor strain are the same. Fragments and mutants of genes or polypeptides are also considered homologous when the gene or polypeptide from which the mutants or fragments are derived is a homologous gene or polypeptide. Also non-native combinations of regulatory sequences and coding sequences are considered homologous as long as the coding sequence is homologous. It follows that the term heterologous herein refers to genes or polypeptides for which donor and acceptor strains do not belong to the same species or variants thereof.

The term “heterologous polypeptide” is defined herein as a polypeptide, which is not native to the fungal cell, a native polypeptide in which modifications have been made to alter the native sequence, or a native polypeptide whose expression is quantitatively altered as a result of a manipulation of the fungal cell by recombinant DNA techniques. For example, a native polypeptide may be recombinantly produced by, e.g., placing the coding sequence under the control of the promoter of the present invention to enhance expression of the polypeptide, to expedite export of a native polypeptide of interest outside the cell by use of a signal sequence, and to increase the copy number of a gene encoding the polypeptide normally produced by the cell.

According to a preferred aspect of the invention, the polypeptide is a peptide hormone or variant thereof, an enzyme, or an intracellular protein.

The intracellular polypeptide may be a protein involved in secretion process, a protein involved in a folding process, a peptide amino acid transporter, a glycosylation factor, a receptor or portion thereof, an antibody or portion thereof, or a reporter protein. Preferably, the intracellular protein is a chaperone or transcription factor. An example of this is described in Appl Microbiol Biotechnol. 1998 October; 50(4):447-54 (“Analysis of the role of the gene bipA, encoding the major endoplasmic reticulum chaperone protein in the secretion of homologous and heterologous proteins in black Aspergilli. Punt P J, van Gemeren I A, Drint-Kuijvenhoven J, Hessing J G, van Muijlwijk-Harteveld G M, Beijersbergen A, Verrips C T, van den Hondel C A). This can be used for example to improve the efficiency of a host cell as protein producer if this coding sequence, such as a chaperone or transcription factor, was known to be a limiting factor in protein production. Another preferred intracellular polypeptide is an intracellular enzyme, such as amadoriase, catalase, acyl-CoA oxidase, linoleate isomerase, trans-2-enoyl-ACP reductase, trichothecene 3-O-acetyltransferase, alcohol dehydrogenase, carnitine racemase, D-mandelate dehydrogenase, enoyl CoA hydratase, fructosyl amine oxygen oxidoreductase, 2-hydroxyhepta-2,4-diene-1,7-dioate isomerase, NADP-dependent malate dehydrogenase, oxidoreductase, quinone reductase. Other intracellular enzymes are ceramidases, epoxide hydrolases aminopeptidases, acylases, aldolase, hydroxylase, aminopeptidases.

According to another preferred aspect of the invention, the polypeptide is secreted extracellularly. Preferably, the extracellular polypeptide is an enzyme. Examples of extracellular enzymes are cellulases such as endoglucanases, β-glucanases, cellobiohydrolases or β-glucosidases; hemicellulases or pectinolytic enzymes such as xylanases, xylosidases, mannanases, galactanases, galactosidases, pectin methyl esterases, pectin lyases, pectate lyases, endo-polygalacturonases, exopolygalacturonases rhamnogalacturonases, arabanases, arabinofuranosidases, arabinoxylan hydrolases, galacturonases, lyases; amylolytic enzymes; phosphatases such as phytases, esterase such as lipases, proteolytic enzyme, such as proteases, peptidases, oxidoreductases such as oxidases, transferases, or isomerases; peroxidases such as ligninases.

The coding sequence comprised in the DNA construct according to the invention may also encode an enzyme involved in the synthesis of a primary or secondary metabolite, such as organic acids, carotenoids, (beta-lactam) antibiotics, and vitamins.

Such metabolite may be considered as a biological compound according to the present invention.

The coding sequence encoding a polypeptide of interest may be obtained from any prokaryotic, eukaryotic, or other source. Preferably, the coding sequence and promoter associated with it are homologous to the host cell, resulting in a recombinant host cell being a self-clone.

According to an embodiment of the invention, the coding sequence in the DNA construct according to the invention may be a variant, optimized sequence comprising an optimized terminator sequence, such as for example described in WO2006 077258.

The coding sequence may be a partly synthetic nucleic acid sequence or an entirely synthetic nucleic acid sequence. The coding sequence may be optimized in its codon use, preferably according to the methods described in WO2006/077258 and/or WO2008/000632, which are herein incorporated by reference. WO2008/000632 addresses codon-pair optimization. Codon-pair optimisation is a method wherein the nucleotide sequences encoding a polypeptide have been modified with respect to their codon-usage, in particular the codon-pairs that are used, to obtain improved expression of the nucleotide sequence encoding the polypeptide and/or improved production of the encoded polypeptide. Codon pairs are defined as a set of two subsequent triplets (codons) in a coding sequence.

Alternatively, the coding sequence may code for the expression of an antisense RNA and/or an RNAi (RNA interference) construct. An example of expressing an antisense-RNA is shown in Appl Environ Microbiol. 2000 February; 66(2):775-82. (Characterization of a foldase, protein disulfide isomerase A, in the protein secretory pathway of Aspergillus niger. Ngiam C, Jeenes D J, Punt P J, Van Den Hondel C A, Archer D B) or (Zrenner R, Willmitzer L, Sonnewald U. Analysis of the expression of potato uridinediphosphate-glucose pyrophosphorylase and its inhibition by antisense RNA. Planta. (1993);190(2):247-52.) Partial, near complete, or complete inactivation of the expression of a gene is useful for instance for the inactivation of genes controlling undesired side branches of metabolic pathways, for instance to increase the production of specific secondary metabolites such as (beta-lactam) antibiotics or carotenoids. Complete inactivation is also useful to reduce the production of toxic or unwanted compounds (chrysogenin in Penicillium; Aflatoxin in Aspergillus: MacDonald K D et al,: heterokaryon studies and the genetic control of penicillin and chrysogenin production in Penicillium chrysogenum. J Gen Microbial. (1963) 33:375-83). Complete inactivation is also useful to alter the morphology of the organism in such a way that the fermentation process and down stream processing is improved.

Another embodiment of the invention relates to the extensive metabolic reprogramming or engineering of a fungal cell. Introduction of complete new pathways and/or modification of unwanted pathways will provide a cell specifically adapted for the production of a specific compound such as a protein or a metabolite.

In the methods of the present invention, when the coding sequence codes for a polypeptide, said polypeptide may also include a fused or hybrid polypeptide in which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide. Techniques for producing fusion polypeptides are known in the art, and include, ligating the coding sequences encoding the polypeptides so that they are in frame and expression of the fused polypeptide is under control of the same promoter(s) and terminator. The hybrid polypeptide may comprise a combination of partial or complete polypeptide sequences obtained from at least two different polypeptides wherein one or more may be heterologous to the fungal cell.

### Control Sequences

The DNA constructs of the present invention may comprise one or more control sequences, in addition to the promoter DNA sequence, which direct the expression of the coding sequence in a suitable host cell under conditions compatible with the control sequences. Expression will be understood to include any step involved in the production of the polypeptide including, but not limited to, transcription, post-transcriptional modification, translation, post-translational modification, and secretion. One or more control sequences may be native to the coding sequence or to the host. Alternatively, one or more control sequences may be replaced with one or more control sequences foreign to the coding sequence for improving expression of the coding sequence in a host cell.

The term “control sequences” is defined herein to include all components, which are necessary or advantageous for the expression of a coding sequence, including the promoter according to the invention. Each control sequence may be native or foreign to the nucleic acid sequence encoding the polypeptide. Such control sequences include, but are not limited to, a leader, an optimal Kozak or translation initiation sequence (Kozak, 1991, J. Biol. Chem. 266:19867-19870) such as for example described in WO2006/077258, a polyadenylation sequence, a propeptide sequence, a signal peptide sequence, an upstream activating sequence, the promoter according to the invention including variants, fragments, and hybrid and tandem promoters derived thereof and a transcription terminator. At a minimum, the control sequences include transcriptional and translational stop signals and (part of) the promoter according to the invention. The control sequences may be provided with linkers for the purpose of introducing specific restriction sites facilitating ligation of the control sequences with the coding region of the coding sequence.

The control sequence may be a suitable transcription terminator sequence, i.e. a sequence recognized by a host cell to terminate transcription. The terminator sequence is in operative association with the 3′ terminus of the coding sequence. Any terminator, which is functional in the host cell of choice may be used in the present invention.

Preferred terminators for filamentous fungal host cells are obtained from the genes for A. oryzae TAKA amylase, A. niger glucoamylase, A. nidulans anthranilate synthase, A. niger alpha-glucosidase, trpC gene, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al, 1992, supra.

The control sequence may also be a suitable leader sequence, i.e. a 5′ untranslated region of a mRNA which is important for translation by the host cell. The leader sequence is in operative association with the 5′ terminus of the nucleic acid sequence encoding the polypeptide. Any leader sequence that is functional in the host cell of choice may be used in the present invention.

Preferred leaders for filamentous fungal host cells are obtained from the genes for A. oryzae TAKA amylase, A. nidulans triose phosphateisomerase and A. niger glaA.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence in operative association with the 3′ terminus of the nucleic acid sequence and which, when transcribed, is recognized by the host cell as a signal to add polyadenosine residues to transcribed mRNA. Any polyadenylation sequence, which is functional in the host cell of choice may be used in the present invention.

Preferred polyadenylation sequences for filamentous fungal host cells are obtained from the genes for A. oryzae TAKA amylase, A. niger glucoamylase, A. nidulans anthranilate synthase, Fusarium oxysporum trypsin-like protease, and A. niger alpha-glucosidase.

Useful polyadenylation sequences for yeast host cells are described by Guo and Sherman, 1995, Molecular Cellular Biology 15: 5983-5990.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Examples of suitable signal peptide coding regions for filamentous fungal host cells are the signal peptide coding regions obtained from the genes for A. oryzae TAKA amylase, A. niger neutral amylase, A. ficuum phytase, A. niger glucoamylase, A. niger endoxylanase, Rhizomucor miehei aspartic proteinase, Humicola insolens cellulase, and Humicola lanuginosa lipase.

Useful signal peptides for yeast host cells are obtained from the genes for Saccharomyces cerevisiae alpha-factor and Saccharomyces cerevisiae invertase.

Other useful signal peptide coding regions are described by Romanoset al., 1992, supra.

The control sequence may also be a propeptide coding region that codes for an amino acid sequence positioned at the amino terminus of a polypeptide. The resultant polypeptide is known as a proenzyme or propolypeptide (or a zymogen in some cases).

A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, Myceliophthora thermophila laccase (WO 95/33836) and A. niger endoxylanase (endo1).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences, which allow gene amplification. Examples of regulatory sequences are in eukaryotic systems, are the dihydrofolate reductase genes, which are amplified in the presence of methotrexate, and the metallothionein genes, which are amplified with heavy metals.

Important can be removal of creA binding sites (carbon catabolite repression as described earlier in EP 673 429), change of pacC and areA (for pH and nitrogen regulation).

### Expression Vectors

The present invention also relates to recombinant expression vectors comprising a DNA construct (comprising a coding sequence in operative association with a promoter DNA sequence according to the invention). Optionally, the at least two DNA constructs of the present invention (comprising a coding sequence in operative association with a promoter DNA sequence) are comprised in a single DNA construct, which single DNA construct is comprised in a recombinant expression vector.

Preferably at least one DNA construct of the present invention (comprising a coding sequence in operative association with a promoter DNA sequence) is present on a vector. The vector is introduced into a host cell so that it is maintained as a chromosomal integrant and/or as a self-replicating extra-chromosomal vector.

The recombinant expression vector may be any vector (e.g., a plasmid or virus), which can be conveniently subjected to recombinant DNA procedures and can bring about the expression of the coding sequence. The choice of the vector will typically depend on the compatibility of the vector with the host cell into which the vector is to be introduced. The vectors may be linear or closed circular plasmids. The skilled person knows, using general knowledge in the art, how to select a suitable and convenient vector.

The vector may be an autonomously replicating vector, i.e., a vector, which exists as an extrachromosomal entity, the replication of which is independent of chromosomal replication, e.g., a plasmid, an extrachromosomal element, a minichromosome, or an artificial chromosome. For autonomous replication, the vector may comprise an origin of replication enabling the vector to replicate autonomously in the host cell in question. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its functioning temperature-sensitive in the host cell (see, e.g., Ehrlich, 1978, Proceedings of the National Academy of Sciences USA 75:1433). An example of an autonomously maintained cloning vector in a filamentous fungus is a cloning vector comprising the AMA1-sequence. AMA1 is a 6.0-kb genomic DNA fragment isolated from A. nidulans, which is capable of Autonomous Maintenance in Aspergillus (see e.g. Aleksenko and Clutterbuck (1997), Fungal Genet. Biol. 21: 373-397).

Alternatively, the vector may be one which, when introduced into the host cell, is integrated into the genome and replicated together with the chromosome(s) into which it has been integrated. An example of such integrative system is described in EP0357127B1. Furthermore, a single vector or plasmid or two or more vectors or plasmids which together contain the total DNA to be introduced into the genome of the host cell, or a transposon may be used.

The vectors of the present invention preferably contain one or more selectable markers, which permit easy selection of transformed cells. The host may be co-transformed with at least two vectors, one comprising the selection marker. A selectable marker is a gene the product of which provides for biocide or viral resistance, resistance to heavy metals, prototrophy to auxotrophs, and the like. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents thereof. Marker conferring resistance against e.g. phleomycin, hygromycin B or G418 can also be used. Preferred for use in an Aspergillus cell are the amdS and pyrG genes of A. nidulans or A. oryzae and the bar gene of Streptomyces hygroscopicus. The amdS marker gene is preferably used applying the technique described in EP 635 574 or WO 97/0626, which enables the development of selection marker free recombinant hosts cells that can be re-transformed using the same selection marker gene. A preferred selection marker gene is the A. nidulans amdS coding sequence fused to the A. nidulans gpdA promoter (EP635 574). AmdS genes from other filamentous fungus may also be used (WO 97/06261).

For integration into the host cell genome, the vector may rely on the promoter sequence and/or coding sequence encoding the polypeptide or any other element of the vector for stable integration of the vector into the genome by homologous or non-homologous recombination. Alternatively, the vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integration elements should preferably contain a sufficient number of nucleic acids, preferably at least 30 bp, preferably at least 50 bp, preferably at least 0.1 kb, even preferably at least 0.2 kb, more preferably at least 0.5 kb, even more preferably at least 1 kb, most preferably at least 2 kb, which share a high percentage of identity with the corresponding target sequence to enhance the probability of homologous recombination. Preferably, the efficiency of targeted integration into the genome of the host cell, i.e. integration in a predetermined target locus, is increased by augmented homologous recombination abilities of the host cell. Such phenotype of the cell preferably involves a deficient ku70 gene as described in WO2005/095624. WO2005/095624 discloses a preferred method to obtain a filamentous fungal cell comprising increased efficiency of targeted integration. The integration elements may be any sequence that is homologous with the target sequence in the genome of the host cell. Furthermore, the integration elements may be non-encoding or encoding nucleic acid sequences. In order to promote targeted integration, the cloning vector is preferably linearized prior to transformation of the host cell. Linearization is preferably performed such that at least one but preferably either end of the cloning vector is flanked by sequences homologous to the target locus.

Preferably, the integration elements in the cloning vector that are homologous to the target locus are derived from a highly expressed locus, meaning that they are derived from a gene which is capable of high expression level in the fungal host cell. A gene capable of high expression level, i.e. a highly expressed gene, is herein defined as a gene whose mRNA can make up at least 0.5% (w/w) of the total cellular mRNA, e.g. under induced conditions, or alternatively, a gene whose gene product can make up at least 1% (w/w) of the total cellular protein, or, in case of a secreted gene product, can be secreted to a level of at least 0.1 g/l (as described in EP 357 127 B1). A number of preferred highly expressed fungal genes are given by way of example: the amylase, glucoamylase, alcohol dehydrogenase, xylanase, glyceraldehyde-phosphate dehydrogenase or cellobiohydrolase genes from Aspergilli or Trichoderma. Most preferred highly expressed genes for these purposes are a glucoamylase gene, preferably an A. niger glucoamylase gene, an A. oryzae TAKA-amylase gene, an A. nidulans gpdA gene, the locus of a sequence selected from the set of SEQ ID NO's:1 to 4 and 13 to 55 or the locus of a gene listed in Table 1, the A. niger locus of a sequence selected from the set of SEQ ID NO's:13 to 55 or the A. niger locus of a gene listed in Table 1, or a Trichoderma reesei cellobiohydrolase gene.

Alternatively, the vector may be integrated into the genome of the host cell by non-homologous recombination.

More than one copy of a nucleic acid sequence encoding a polypeptide may be inserted into the host cell to increase production of the gene product. This can preferably be performed by integrating into its genome copies of the DNA sequence, more preferably by targeting the integration of the DNA sequence at a highly expressed locus, for example at a glucoamylase locus or at the locus of a sequence selected from the set of SEQ ID NO's:1 to 4 and 13 to 55 or at the locus of a gene listed in Table 1. Alternatively, this can be performed by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent. To increase the number of copies of the DNA sequence to be over expressed even more, the technique of gene conversion as described in WO98/46772 can be used.

The procedures used to ligate the elements described above to construct the recombinant expression vectors of the present invention are well known to one skilled in the art (see, e.g., Sambrook et al., supra).

### Transformation

The introduction of an expression vector or a nucleic acid construct into a cell is performed using commonly known techniques. It may involve a process consisting of protoplast formation, transformation of the protoplasts, and regeneration of the cell wall in a manner known per se. Suitable procedures for transformation of Aspergillus cells are described in EP 238 023 and Yelton et al., 1984, Proceedings of the National Academy of Sciences USA 81: 1470-1474. Suitable procedures for transformation of Aspergillus and other filamentous fungal host cells using Agrobacterium tumefaciens are described in e.g. Nat. Biotechnol. 1998 September; 16(9):839-42. Erratum in: Nat Biotechnol 1998 November; 16(11):1074. Agrobacterium tumefaciens-mediated transformation of filamentous fungi. de Groot M J, Bundock P, Hooykaas P J, Beijersbergen A G. Unilever Research Laboratory Vlaardingen, The Netherlands. A suitable method of transforming Fusarium species is described by Malardier et al., 1989, Gene 78: 147156 or in WO 96/00787. Other methods can be applied such as a method using biolistic transformation as described in: Biolistic transformation of the obligate plant pathogenic fungus, Erysiphe graminis f.sp. hordei. Christiansen S K, Knudsen S, Giese H. Curr Genet. 1995 December; 29(1):100-2. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

The invention further relates to a method to prepare the recombinant host cell according to the invention, said method comprising:
(a) providing at least two DNA constructs, each DNA construct comprising a coding sequence in operative association with a promoter DNA sequence, wherein the at least two DNA constructs comprise at least two distinct promoter DNA sequences and wherein the coding sequences comprised in said DNA constructs encode related polypeptides,
(b) providing a suitable host cell, and
(c) transforming said host cell with said DNA constructs.

Optionally, said two DNA constructs, are comprised in a single construct.

Optionally, at least one DNA construct is present on a vector.

According to a preferred embodiment, the transformation step is performed by at least two separate transformation events. A transformation event is herein defined as the procedure of transformation by introduction of a DNA construct in a parental host cell and isolation of transformed offspring of the parental cell.

### Expression

The invention further relates to a method for expression of a coding sequence in a suitable host cell. The method comprising the following steps:
(a) providing at least two DNA constructs, each DNA construct comprising a coding sequence in operative association with a promoter DNA sequence, wherein the at least two DNA constructs comprise at least two distinct promoter DNA sequences and wherein the coding sequences comprised in said DNA constructs encode related polypeptides,
(b) providing a suitable host cell,
(c) transforming said host cell with said DNA constructs,
(d) culturing said host cell under conditions conducive to expression of the coding sequence.

Optionally, said two DNA constructs, are comprised in a single construct. Optionally, at least one DNA construct is present on a vector.

According to a preferred embodiment, the transformation step is performed by at least two separate transformation events.

The invention further relates to a method for expression of coding sequence by culturing a recombinant host cell according to the invention under conditions conducive to expression of the coding sequence.

### Production

The invention further relates to a method for the production of a polypeptide, comprising:
(a) culturing a recombinant host cell according to the invention under conditions conducive to expression of the polypeptide,
(b) optionally recovering the polypeptide from the culture broth, and
(c) optionally purifying the polypeptide.

The invention also relates to a method for the production of a metabolite, comprising:
(a) culturing a recombinant host cell according to the invention under conditions conducive to production of the metabolite,
(b) optionally recovering the metabolite from the culture broth, and
(c) optionally purifying the metabolite.

In the production methods of the present invention, the cells are cultivated in a nutrient medium suitable for production of the polypeptide or metabolite using methods known in the art. Examples of cultivation methods which are not construed to be limitations of the invention are submerged fermentation, surface fermentation on solid state and surface fermentation on liquid substrate. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the coding sequence to be expressed and/or the polypeptide to be isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide or metabolite is secreted into the nutrient medium, the polypeptide or metabolite can be recovered directly from the medium. If the polypeptide or metabolite is not secreted, it can be recovered from cell lysates.

The polypeptides may be detected using methods known in the art that are specific for the polypeptides. These detection methods may include use of specific antibodies, formation of an enzyme product, or disappearance of an enzyme substrate.

The resulting polypeptide or metabolite may be recovered by methods known in the art. For example, the polypeptide or metabolite may be recovered from the nutrient medium by conventional procedures including, but not limited to, centrifugation, filtration, extraction, spray-drying, evaporation, or precipitation.

Polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

### Modulation of Expression

The present invention also relates to nucleic acid constructs comprising a promoter DNA sequence comprising a nucleotide sequence selected from the set consisting of: SEQ ID NO's:1 to 4 and 13 to 55 and the promoter DNA sequences of the genes listed in Table 1, for altering the expression of a coding sequence encoding a compound of interest, which is endogenous to a fungal host cell. The constructs may contain the minimal number of components necessary for altering expression of the endogenous gene.

According to a preferred embodiment, the nucleic acid constructs contain (a) a targeting sequence, (b) a promoter DNA sequence comprising a nucleotide sequence selected from the set consisting of: a sequence of SEQ ID NO's:1 to 4 and 13 to 55 and the promoter DNA sequences of the genes listed in Table 1, (c) an exon, and (d) a splice-donor site. Upon introduction of the nucleic acid construct into a cell, the construct integrates by homologous recombination into the cellular genome at the endogenous gene site. The targeting sequence directs the integration of elements (a)-(d) into the endogenous gene such that elements (b)-(d) are in operative association with the endogenous gene.

According to another embodiment, the nucleic acid constructs contain (a) a targeting sequence, (b) a promoter DNA sequence comprising a nucleotide sequence selected from the set consisting of: a sequence of SEQ ID NO's:1 to 4 and 13 to 55 and the promoter DNA sequences of the genes listed in Table 1, (c) an exon, (d) a splice-donor site, (e) an intron, and (f) a splice-acceptor site, wherein the targeting sequence directs the integration of elements (a)-(f) such that elements (b)-(f) are in operative association with the endogenous gene. However, the constructs may contain additional components such as a selectable marker. The selectable markers that can be used are those described earlier herein.

In both embodiments, the introduction of these components results in production of a new transcription unit in which expression of the endogenous gene is altered. In essence, the new transcription unit is a fusion product of the sequences introduced by the targeting constructs and the endogenous gene. According to an embodiment in which the endogenous gene is altered, the gene is activated. According to this embodiment, homologous recombination is used to replace, disrupt, or disable the regulatory region normally associated with the endogenous gene of a parent cell through the insertion of a regulatory sequence, which causes the gene to be expressed at higher levels than evident in the corresponding parent cell.

The targeting sequence can be within the endogenous gene, immediately adjacent to the gene, within an upstream gene, or upstream of and at a distance from the endogenous gene. One or more targeting sequences can be used. For example, a circular plasmid or DNA fragment preferably employs a single targeting sequence, while a linear plasmid or DNA fragment preferably employs two targeting sequences.

The constructs further contain one or more exons of the endogenous gene. An exon is defined as a DNA sequence, which is copied into RNA and is present in a mature mRNA molecule such that the exon sequence is in-frame with the coding region of the endogenous gene. The exons can, optionally, contain DNA, which encodes one or more amino acids and/or partially encodes an amino acid. Alternatively, the exon contains DNA which corresponds to a 5′ non-encoding region. Where the exogenous exon or exons encode one or more amino acids and/or a portion of an amino acid, the nucleic acid construct is designed such that, upon transcription and splicing, the reading frame is in-frame with the coding region of the endogenous gene so that the appropriate reading frame of the portion of the mRNA derived from the second exon is unchanged. The splice-donor site of the constructs directs the splicing of one exon to another exon. Typically, the first exon lies 5′ of the second exon, and the splice-donor site overlapping and flanking the first exon on its 3′ side recognizes a splice-acceptor site flanking the second exon on the 5′ side of the second exon. A splice-acceptor site, like a splice-donor site, is a sequence, which directs the splicing of one exon to another exon. Acting in conjunction with a splice-donor site, the splicing apparatus uses a splice-acceptor site to effect the removal of an intron.

A preferred strategy for altering the expression of a given DNA sequence comprises the deletion of the given DNA sequence and/or replacement of the endogenous promoter sequence of the given DNA sequence by a modified promoter DNA sequence, such as a promoter according to the invention. The deletion and the replacement are preferably performed by the gene replacement technique described in EP 0 357 127. The specific deletion of a gene and/or promoter sequence is preferably performed using the amdS gene as selection marker gene as described in EP 635 574. By means of counterselection on fluoracetamide media as described in EP 635 574, the resulting strain is selection marker free and can be used for further gene modifications.

Alternatively or in combination with other mentioned techniques, a technique based on in vivo recombination of cosmids in E. coli can be used, as described in: A rapid method for efficient gene replacement in the filamentous fungus A. nidulans (2000) Chaveroche, M-K., Ghico, J-M. and d'Enfert C; Nucleic acids Research, vol 28, no 22. This technique is applicable to other filamentous fungi like for example A. niger.

The invention described and claimed herein is not to be limited in scope by the specific embodiments herein disclosed, since these embodiments are intended as illustrations of several aspects of the invention. Any equivalent embodiments and/or combinations of preferred aspects of the invention are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### EXAMPLES

### Experimental Information

### Strains

WT1: This A. niger strain is used as a wild-type strain. This strain is deposited at the CBS Institute under the deposit number CBS 513.88.

WT2: This A. niger strain is a WT1 strain comprising a deletion of the gene encoding glucoamylase (g/aA). WT2 was constructed by using the “MARKER-GENE FREE” approach as described in EP 0 635 574 B1. In this patent it is extensively described how to delete g/aA specific DNA sequences in the genome of CBS 513.88. The procedure resulted in a MARKER-GENE FREE Δg/aA recombinant A. niger CBS513.88 strain, possessing finally no foreign DNA sequences at all.

BFRM 44: This Pycnoporus cinnabarinus strain is used in example 9 and is available from the Banque de Resources Fongiques de Marseille, Marseiile, France under deposit number BRFM44.

### Glucoamylase Activity Assay

The glucoamylase activity was determined using p-Nitrophenyl α-D-glucopyranoside (Sigma) as described in WO 98/46772.

### Example 1

### Construction of a DNA Construct Comprising a Promoter According to the Invention in Operative Association with a Coding Sequence

This example describes the construction of an expression construct under control of a promoter according to the invention. The coding sequence or reporter construct used here is the glaA gene encoding the A. niger CBS 513.88 glucoamylase enzyme. Glucoamylase is used as the reporter enzyme to be able to measure the activity of the promoter according to the invention.

1.1 Description of an Integrative Glucoamylase Expression Vector (pGBTOPGLA)

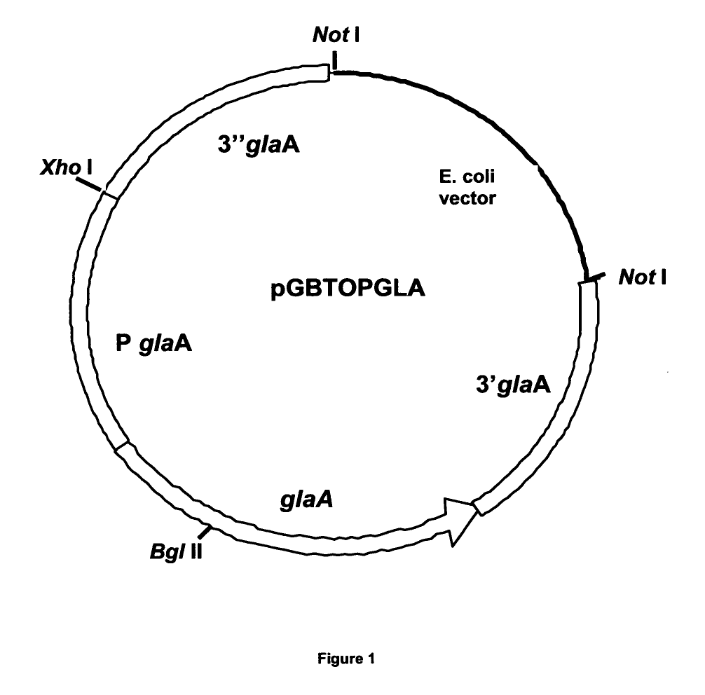

The glucoamylase promoter and the glucoamylase encoding gene glaA from A. niger were cloned into the expression vector pGBTOP-8, which is described in WO99/32617. The cloning was performed according known principles and to routine cloning techniques and yielded plasmid pGBTOPGLA (see FIG. 1). In essence, this expression vector comprises the glucoamylase promoter, coding sequence and terminator region, flanked by the 3′ and 3″ glaA targeting sites in an E. coli vector.

1.2 Construction of an Integrative Glucoamylase Expression Vector with a Multiple Cloning Site MCS (pGBTOPGLA-2)

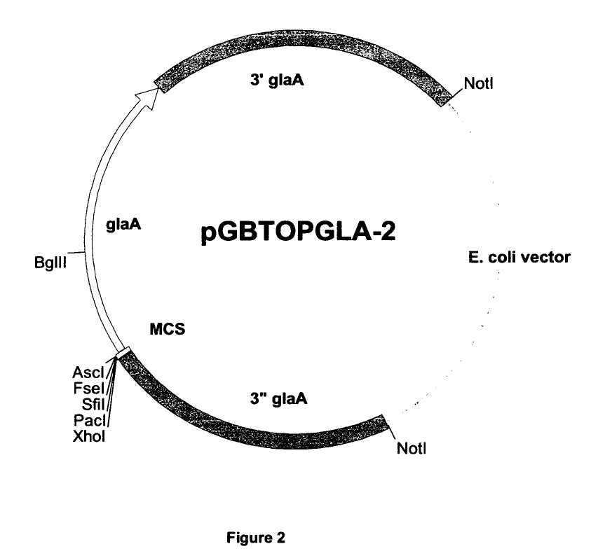

With PCR methods known to the skilled person (Sambrook et al., supra), using 1 ng of pGBTOPGLA as template, a PCR fragment was generated containing part of the g/aA coding sequence and flanked with XhoI and BglII restriction sites. This fragment was digested with XhoI and BglII and introduced in XhoI and BglII digested vector pGBTOPGLA, resulting in vector pGBTOPGLA-2 (see FIG. 2). The sequence of the introduced PCR fragment comprising a MCS and part of the glaA coding sequence was confirmed by sequence analysis.

1.3 Construction of an Integrative Expression Vector with the Promoter According To the Invention in Operative Association with the Glucoamylase Coding Sequence (pGBTOPGLA-3)

Genomic DNA of strain CBS513.88 was sequenced and analysed. With PCR methods known to the skilled person (Sambrook et al., supra) using:
a 5′-PCR primer containing nucleotides 1 to 20 of a sequence selected from the set of: SEQ ID NO's:13 to 55 and the promoter DNA sequences of the genes listed in Table 1, flanked at the 5′-end with a XhoI restriction site, and
a 3′-PCR primer containing nucleotides 1477 to 1497 (in some cases around 2000 nucleotides) of the sequence selected immediately here above, flanked at the 3′-end with an Ascl restriction site, and
genomic DNA of strain CBS513.88 as template,

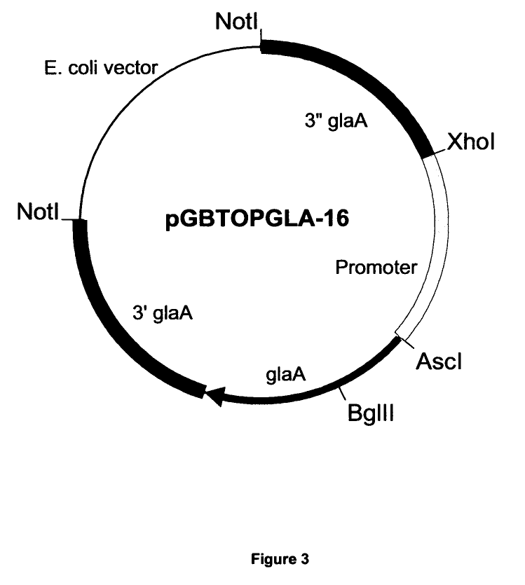

appropriate restriction sites were attached to the promoter according to the invention by PCR amplification. The resulting fragments of approximately 1.5-2 kb, comprising the sequence of one of SEQ ID NO's:13 to 55 or of one of the promoter DNA sequences of the genes listed in Table 1, were digested with Ascl and XhoI and introduced in an Ascl and XhoI digested vector pGBTOPGLA-2, resulting for example in vector pGBTOPGLA-16 (for general layout of vectors see FIG. 3) and other vectors as for example listed in Table 2.

TABLE 2
Expression constructs with a number of different promoters
for glucoamylase glaA expression in A. niger

| | | | Coding |
|---|---|---|---|
| Plasmid name | Promoter | SEQ ID NO: | sequence |
| pGBTOPGLA-1 | glaA | — | GlaA |
| pGBTOPGLA-16 | glaA with modified | 16 | GlaA |
| | translation initiation site | | |
| pGBTOPGLA-17 | amyB with modified | 17 | GlaA |
| | translation initiation site | | |
| pGBTOPGLA-18 | gpdA | 18 | GlaA |
| pGBTOPGLA-19 | An18g04220 | 19 | GlaA |

The sequences of the introduced PCR fragments comprising the promoters according to the invention were confirmed by sequence analysis.

### Example 2

### Fungal Host Cell Transformed with the DNA Construct

In the following example, an expression construct is introduced in a fungal host cell by transformation.

In order to introduce additional pGBTOPGLA-1, and pGBTOPGLA-16 to -19 vectors in WT1, to increase the glaA copy number, a transformation and subsequent transformant selection was carried out as described in WO98/46772 and WO99/32617. In brief, linear DNA of the above mentioned pGBTOPGLA vectors was isolated and co-transformed with an amdS selectable marker-gene containing vector, which is designated pGBAAS-1 (constructed as described in EP 635574B1). Both types of vectors comprise two DNA domains homologous to the glaA locus of A. niger host strain to direct targeting to the 3′-3″ terminator locus of glaA in WT1. Transformants were selected on acetamide media and colony purified according standard procedures. Spores were plated on fluoro-acetamide media to select strains, which lost the amdS marker. Growing colonies were diagnosed for integration at the glaA locus and copy number. Transformants of pGBTOPGLA-1, -16, -17, -18 and -19 with an additional copy of glaA were selected.

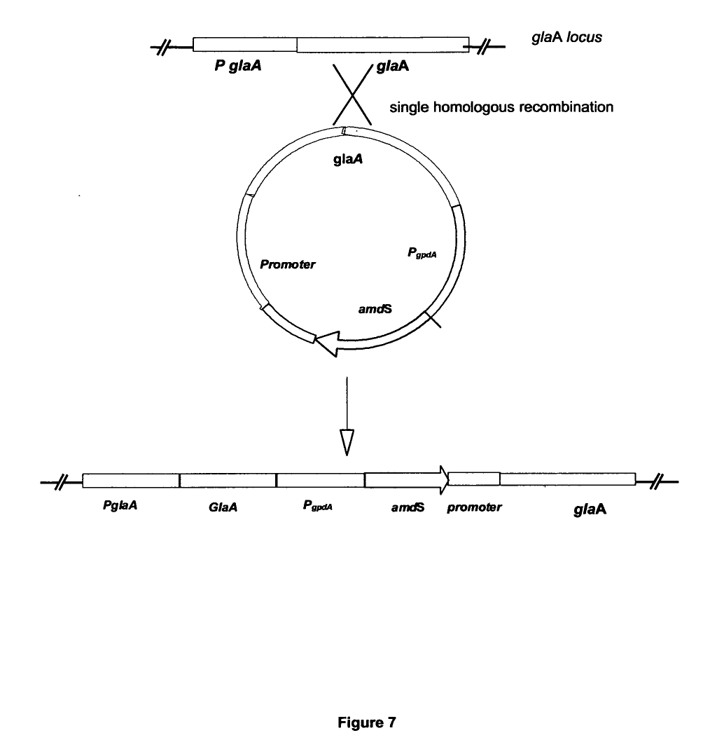

Alternatively, a circular construct as depicted in FIG. 7 can be used to integrate into the genome at the glaA coding sequence of WT1. Additionally, the selectable marker gene and the gene of interest controlled by a promoter according to the invention can be on a single construct. Example of this vector and how to use in transformation can be found in WO99/32617.

### Example 3

### Introduction of an Additional glaA Gene Under Control of a Promoter According to the Invention in the Fungal Host Cell

To alter and increase the expression level of a given gene in a host cell, additional copies of glucoamylase operatively linked to a promoter according to the invention can be introduced in a given host cell. In this example, a promoter of the invention operatively linked with the glaA coding sequence is introduced next to endogenously present glucoamylase encoding glaA gene in a fungal host cell. In the following example, the activity of a promoter of the invention is measured by measuring the activity of the reporter (glucoamylase) in selected transformants. Therefore, the glucoamylase activity is determined in the culture broth.

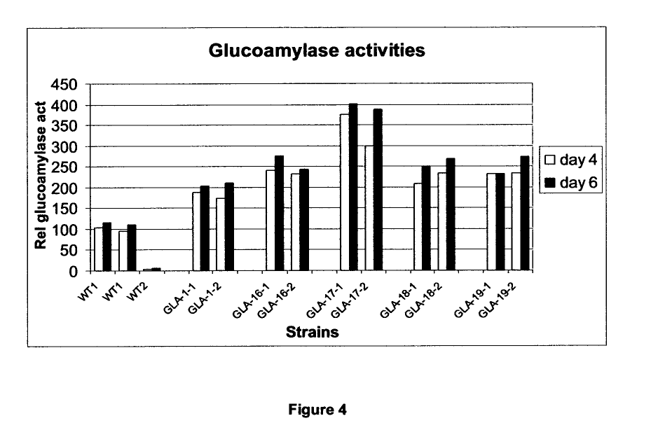

The selected pGBTOPGLA-1, 16, -17, -18, -19 transformants of WT1 and both strains WT1 and WT2 were used to perform shake flask experiments in 100 ml of the medium as described in EP 635 574 B1 at 34° C. and 170 rpm in an incubator shaker using a 500 ml baffled shake flask. After 4 and 6 days of fermentation, samples were taken to determine the glucoamylase activity, as described above. The glucoamylase activity in the selected pGBTOPGLA-1, -16, -17, -18, -19 transformants of WT1 was increased compared to WT1 after either four or six days of culture (FIG. 4).

Surprisingly, it was shown that introducing a second gIaA gene copy under the control of another promoter than glaA increased expression more than could be expected based on the copy number (more than 200%). Also using a modified gIaA or amyB promoter, increases larger than expected based on copy number were identified.

Additionally, strains with multiple copies of the pGBTOPGLA constructs showed that the production per gene copy was constant until at least 5 copies for all vectors (data not shown).

### Example 4

### Construction of a Promoter Replacement Construct pGBGEL-pGLAA Comprising a Promoter According to the Invention

To alter the expression level of a given gene in a host cell, a promoter according to the invention can replace the endogenous promoter of said given gene. In this example, a promoter according to the invention replaces the promoter of the glucoamylase encoding glaA gene in a fungal host cell. Example 4, 5 and 6 describe a number of different steps in this process.

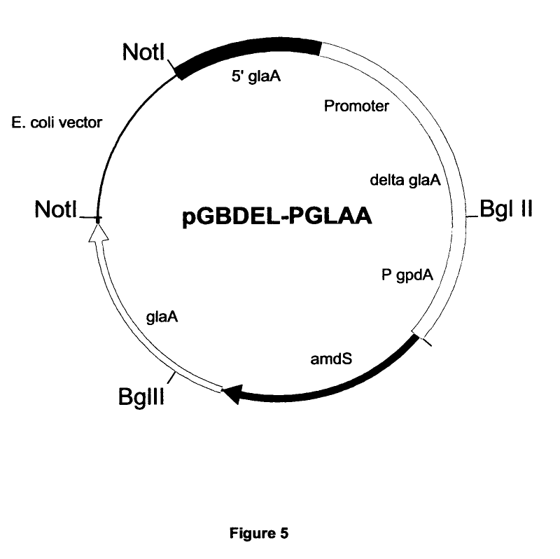

A replacement vector for the glucoamylase promoter was designed according to known principles and constructed according to routine cloning procedures (see FIG. 5). In essence, the glaA promoter replacement vector pGBDEL-PGLAA comprises approximately 1000 by flanking regions of the glaA promoter sequence to be replaced by a promoter according to the invention through homologous recombination at the predestined genomic locus. The flanking regions used here (see FIG. 5) are a 5′ upstream region of the glaA promoter and part of the glaA coding sequence. In addition, the replacement vector contains the A. nidulans bi-directional amdS selection marker, in-between direct repeats. The direct repeats used in this example are part of the glaA coding sequence. The general design of these deletion vectors were previously described in EP635574B1 and WO 98/46772.

### Example 5

### Replacement of the glaA Promoter by a Promoter According to the Invention in the Fungal Host Cell

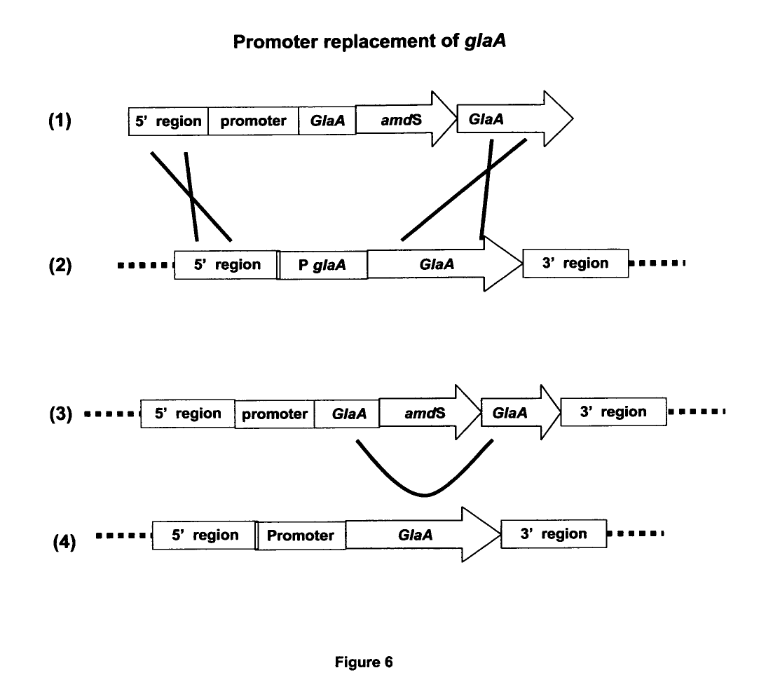

Linear DNA of NotI-digested deletion vector pGBDEL-PGLAA was isolated and used to transform WT 1 (CBS513.88). This linear DNA can integrate into the genome at the glaA locus, thus substituting the glaA promoter region with the construct containing amdS and a promoter according to the invention (see FIG. 6). Transformants were selected on acetamide media and colony purified according to standard procedures. Growing colonies were diagnosed by PCR for integration at the glaA locus. Deletion of the glaA promoter was detectable by amplification of a band, with a size specific for the promoter according to the invention and loss of a band specific for the glaA promoter. Spores were plated on fluoro-acetamide media to select strains, which lost the amdS marker. Candidate strains were tested using Southern analysis for proper deletion of the glucoamylase promoter and replacement by a promoter according to the invention. Strains dPGLAA were selected as representative strains with the glaA promoter replaced by the promoter according to the invention and having a restored functional glaA coding sequence (see FIG. 6).

### Example 6

### Production of the Glucoamylase Polypeptide Encoded by the glaA Coding Sequence Under Control of a Replaced Promoter According to the Invention, in the Fungal Host Cell

The selected dPGLAA strains (proper pGBDEL-PGLAA transformants of WT 1, isolated in example 5) and strain WT 1 were used to perform shake flask experiments in 100 ml of the medium as described in EP 635 574 B1 at 34° C. and 170 rpm in an incubator shaker using a 500 ml baffled shake flask. After 4 and 6 days of fermentation, samples were taken to determine the glucoamylase activity. The glucoamylase activity in the selected dpGLAA transformants of WT1 was altered compared to the one measured for WT 1 after either 4 or six days of fermentation.

### Example 7

### Integration of glaA Genes Under Control of Multiple Promoters According to the Invention in the Fungal Host Cell

To alter and increase the expression level of a given gene in a host cell, multiple additional copies of a given gene operatively linked to various promoters according to the invention can be introduced in a given host cell. In this example, various promoters according to the invention operatively linked with the glaA coding sequence are introduced in a fungal host cell WT2. Example 7 and 8 describe a number of different steps in this process.

In order to introduce combinations of pGBTOPGLA-1, and pGBTOPGLA-16, -17 and -18 vectors in WT2, a cotransformation and subsequent transformant selection was carried out as described in WO98/46772 and WO99/32617. In principle, linear DNA of two of the above mentioned pGBTOPGLA vectors was isolated and co-transformed with an amdS selectable marker-gene containing vector, which is designated pGBAAS-1 (constructed as described in EP 635574B1). Both types of vectors comprise two DNA domains homologous to the glaA locus of A. niger host strain to direct targeting to the 3′-3″ terminator locus of glaA in WT2. Transformants were selected on acetamide media and colony purified according standard procedures. Cotransformants were identified using PCR techniques and colonies were diagnosed for glaA copy number and integration of two different pGBTOPGLA-contructs at the glaA locus. Transformants with 2 glaA copies and a combination of pGBTOPGLA-1/16, pGBTOPGLA-16/17 and pGBTOPGLA-17/18 were selected.

### Example 8

### Production of the Glucoamylase Polypeptide Encoded by the glaA Coding Sequences Under Control of Multiple Promoters of the Invention in a Fungal Host Cell

The selected pGBTOPGLA-1/16, pGBTOPGLA-16/17 and pGBTOPGLA-17/18 strains, isolated in example 7, and strain WT 1 were used to perform shake flask experiments in 100 ml of the medium as described in EP 635 574 B1 at 34° C. and 170 rpm in an incubator shaker using a 500 ml baffeled shake flask. After 4 and 6 days of fermentation, samples were taken to determine the glucoamylase activity. The glucoamylase activity in the selected pGBTOPGLA-16/17 and pGBTOPGLA-17/18 transformants of WT2 were increased compared to the pGBTOPGLA-1/16 transformants and WT 1, measured after either four or six days of fermentation (data not shown). This clearly shows that expression of the glaA gene under control of multiple and non-native promoters can provide increased expression of glaA.

### Example 9

### Differential Production of the Laccase Polypeptide Encoded by the Pycnoporus cinnabarinus Icc3-1 Gene in Pycnoporus cinnabarinus

As depicted in detail below, it was demonstrated that SC3 promoter driven expression of Icc3-1 results in the release of laccase at the periphery of colonies of P. cinnabarinus. In contrast, GPD promoter and laccase promoter driven expression result in the release of laccase in the centre and the middle of the colony, respectively.

By transforming strain BRFM 44 with two expression constructs, laccase was released at the periphery and the middle (SC3 and GPD driven expression of Icc3-1), the periphery and the centre (SC3 and GPD driven expression) and the middle and the centre (Laccase promoter and GPD promoter driven expression) of the colony.

Accordingly, by using combinations of expression constructs multiple parts of the mycelium can be involved in secretion of laccase. This phenomenom is accompanied with an increase in laccase activity in liquid shaken cultures.

### 9.1 Materials and Methods

Cultivation of P. cinnabarinus

The monokaryotic laccase deficient Pycnoporus cinnabarinus strain BRFM 44 (Banque de Resources Fongiques de Marseille, Marseille, France) was routinely grown at 30 DEG C in liquid or solid (1.5% agar) yeast malt medium (YM) containing per liter 10 g glucose, 5 g peptone, 3 g yeast extract, and 3 g malt extract. For laccase production, conditions were used that are optimal for laccase production in wild-type P. cinnabarinus (Lomascolo et al., 2003). Strains were grown in 250 ml minimal medium (MM) either or not containing filter sterilized ethanol in 1 L Erlenmeyer flasks at 250 rpm at 30 DEG C. MM contained per liter: 20 g maltose, 1 g yeast extract, 2.3 g C₄H₄O₆Na₂.2H₂O, 1.84 g (NH₄)2C₄H₄O₆, 1.33 g KH₂PO₄, 0.1 g CaCl₂.2H₂O, 0.5 g MgSO₄, 0.07 g FeSO₄.7H₂O, 0.048 g ZnSO₄.7H₂O, 0.036 g MnSO₄.H₂O, 0.1 g CuSO₄ and 1 ml of a vitamin solution (Tatum et al., 1950).

Transformation of P. cinnabarinus

P. cinnabarinus was transformed as described by Alves A M, Record E, Lomascolo A, Scholtmeijer K, Asther M, Wessels J G, and Wosten H A. Highly efficient production of laccase by the basidiomycete Pycnoporus cinnabarinus, Appl Environ Microbiol. 2004 November; 70(11):6379-84. All steps in the transformation procedure were carried out at 30 DEG C unless stated otherwise. A 15 days-old colony (6-8 cm in diameter) was homogenized in 50 ml YM medium for 1 min in a Waring blendor. After adding the same volume of medium, the homogenate was grown for 24 h at 200 rpm. This culture was again homogenized, diluted twice in YM, and grown for 24 h at 200 rpm. The mycelium was protoplasted in 0.5 M MgSO₄ or 0.5 M sucrose with gentle shaking using 1 mg ml⁻¹ glucanex (Sigma-Aldrich). 1E+7 protoplasts and 5 μg of plasmid DNA were incubated for 15 min on ice. After adding 1 volume of polyethyleneglycol 4000 the mixture was incubated for 5 minutes at room temperature. Protoplasts were regenerated overnight in 2.5 ml regeneration medium (Specht et al., 1988, Exp. Mycol. 12: 357-366). After adding three volumes of YM medium containing 5 μg ml⁻¹ phleomycin and 1% low melting point agarose the mixture was spread on YM agar medium containing 5 μg ml⁻¹ of the antibiotic. Transformants in which a phleomycin resistance cassette was already introduced were retransformed with the same selection cassette by adding 500 μg ml⁻¹ caffeine to the medium in addition to the antibiotic.

### Construction of Laccase Expression Vectors

To express the laccase Icc3-1 gene from P. cinnabarinus behind the SC3 and GPD promoters of S. commune, its coding sequence was amplified by PCR using primers NcoIPyc and BcllPyc (Table 3). This resulted in a fragment with an introduced NcoI site in the start codon and a Bc/I restriction site directly following the stop codon. To express the Icc3-1 gene behind the laccase promoter, the coding sequence was amplified using primers PromoNCOforward and PromoLACreverse (Table 3), resulting in a fragment with an introduced NcoI site a the 5′ end, and a SmaI site immediately following the stop codon. The amplified coding sequences of Icc3-1 were cloned in the expression vector pESC and its derivatives pEGP and pELP, resulting in plasmids pESCL1, pEGPL1, pELPL1, respectively. Plasmid pESC contains a phleomycin resistance cassette (Schuren and Wessels, 1994), in which the internal NcoI site has been deleted. Moreover, it contains the regulatory sequences of the SC3 gene in between which coding sequences can be cloned using NcoI and BamHI sites. The SC3 promoter is contained on a 1.2 kb HinDIII/NcoI fragment, while its terminator consists of a 434 by BamHI/EcoRI fragment. Plasmids pEGP and pELP are derivatives of pESC, in which the SC3 promoter is replaced for HinDIII/NcoI promoter fragments of GPD (700 bp) (Harmsen et al. 1992) and laccase (2.5 kb), respectively. The laccase promoter was isolated as follows. Bg/II digested genomic DNA of P. cinnabarinus was circularized by self-ligation and used as a template for inverse PCR using the primers INVSE and INVASE (Table 3). The resulting 3.5 kb fragment was cloned in XL-TOPO (Invitrogen) resulting in plasmid pPL100. Sequencing confirmed that pPL100 contained a 2.5 kb promoter region. This region was amplified by PCR using primers promol.ACforward and promoNCOrev (Table 3) introducing a HinDIII and a NcoI site at the 5′ and 3′ ends, respectively.

TABLE 3

Primers used for construction of the laccase expression
vectors, the respective primer sequences and the respective
SEQ ID NO:'s.

| | | |
|---|---|---|
| Primer | Sequence | SEQ ID NO: |

| | | |
|---|---|---|
| NcoI Pyc | ttctgaccatggcgaggttccagtc | 5 |
| BclI Pyc | acagtaactgattcagctcagaggtcgctg | 6 |
| PromoNCOforward | accccctctttctgaccatggcgaggttccagtc | 7 |
| PromoLACreverse | taacccgggcgctcagaggtcgctggggtcaagtgc | 8 |
| INVSE | tctgatcatgtcgaggttccagtcc | 9 |
| INVASE | gtcttcaaggacctgcggacagacatc | 10 |
| Promolacforward | accaagcttagatctccgaaccagaaatgc | 11 |
| PromoNCOrev | gactggaacctcgccatggtcagaaagaggggt | 12 |

### Laccase Activity Determination

Laccase activity of P. cinnabarinus strains was monitored on solid YM medium supplemented with 0.2 mM ABTS (2,2′-Azino-bis(3-ethylbenzothiazoline-6-sulfonic acid), Sigma-Aldrich) and 0.1 mM CuSO₄. Laccase activity in the culture medium was determined quantitatively by following the oxidation of 5 mM ABTS at 420 nm (extinction coefficient 36 000 mM⁻¹ cm⁻¹) in the presence of 50 mM Na—K-tartrate pH 4.0. Activity was expressed as nkat ml⁻¹. 1 nkat is defined as the amount of enzyme catalyzing the oxidation of 1 nmol of ABTS per second. Assays were carried out at 30 DEG C in triplicate. Standard deviation did not exceed 10% of the average values.

9.2 Laccase Production in Pycnoporus cinnabarinus

Previously, laccase production in Pycnoporus cinnabarinus was demonstrated according to Alves et al., supra.

The laccase deficient monokaryotic strain BRFM 44 (Banque de Resources Fongiques de Marseille, Marseille, France) of Pycnoporous cinnabarinus was transformed with the native Icc3-1 laccase gene (SEQ ID NO: 4) placed under regulation of the laccase promoter (SEQ ID NO: 3) or that of the SC3 hydrophobin gene (SEQ ID NO: 2) or the glyceraldehyde-3-phosphate dehydrogenase (GPD) gene of Schizophyllum commune (SEQ ID NO: 1). SC3 driven expression resulted in a laccase activity of maximally 107 nkat ml-1 in liquid shaken cultures. This value was about 1.4 and 1.6 times higher in the case of the GPD and laccase promoters, respectively (Table 4). Icc3-1 mRNA and laccase activity were strongly increased in the presence of 25 g L⁻¹ ethanol when Icc3-1 was expressed behind either promoter (Table 3). Laccase production was further increased in transformants expressing the Icc3-1 gene behind the laccase promoter or that of GPD by growing in the presence of 40 g L⁻¹ ethanol. In this case maximal activities were 3900 and 4660 nkat ml⁻¹, respectively, corresponding to 1 and 1.2 g of laccase per liter.

TABLE 4

| | promoter | | |
|---|---|---|---|
| Strain | used | Activity −EtOH | Activity +EtOH |
| BRFM 44 | — | n.d | n.d. |
| S1 | SC3 | 107 | 431 |
| S2 | SC3 | 49 | 138 |
| L12-7 | Icc3-1 | 175 | 1223 |
| L12-8 | Icc3-1 | 20 | 666 |
| G11 | GPD | 60 | 700 |
| G14 | GPD | 145 | 1393 |

Laccase activity (nkat ml⁻¹) in media of 14-day old cultures of recombinant strains of P. cinnabarinus BRFM 44 expressing the laccase gene Icc3-1 behind the laccase promoter (L12-7 & L12-8), or that of the SC3 (S1 & S2) or GPD (G11 and G14) promoter of S. commune. Strains were grown in the presence or absence of 25 g L⁻¹ ethanol. n.d.: not detectable. Experiments were performed in triplicate. The standard deviation was less than 10%.

9.3 Differential Laccase Production in Pycnoporus cinnabarinus

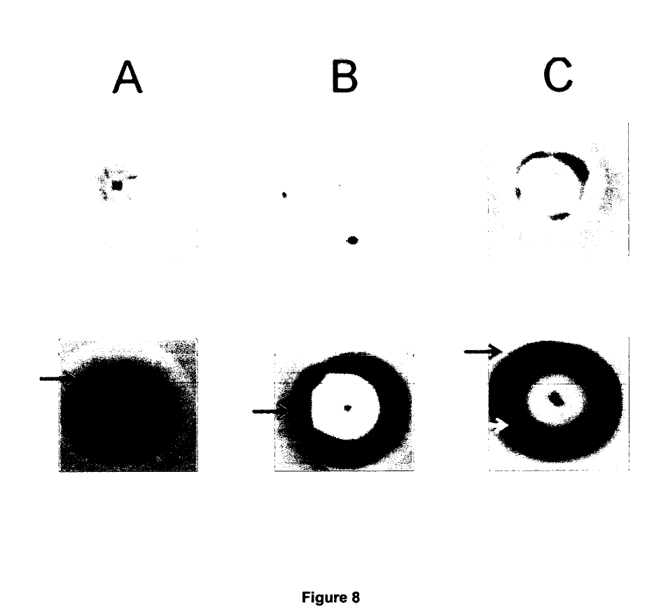

Constructs pESCL1, pEGPL1, pELPL1 expressing the laccase gene Icc3-1 from the SC3, GPD and laccase promoter, respectively, were transformed to the laccase negative monokaryon BRFM 44 of P. cinnabarinus Alves et al., 2004, supra). Laccase producing transformants were selected by adding the substrate ABTS to the medium, which is converted in a green product by the enzyme. Spatial and temporal laccase activity was monitored, i.e. the laccase activity was monitored at various time intervals, and the locatlisation of the laccase activity in the colony was assessed. Two independent transformants of each construct were selected to exclude positioning effects. The presence of a PC membrane in between the agar medium and the colony did not affect the spatial and temporal activity of laccase on media containing ABTS. Colonies with introduced laccase gene(s) under regulation of the GPD promoter showed laccase activity exclusively in the centre of the colony starting at day 6 (FIG. 8). After 10 days of growth activity had extended to the periphery and degradation of the substrate had increased. SC3 driven expression resulted in activity at the periphery of the colony from day 5 on. After 10 days of growth activity was still observed at the periphery and the intensity of the break down product had increased. Strains transformed with the laccase gene regulated by its own promoter showed activity in the middle of the colony. This activity was observed at day 4. After 10 days of growth the activity zone had extended outwards to a degree similar to that of the extension of the periphery of the colony. From these results we conclude that spatial and temporal activity of laccase in the medium depends on the promoter used.

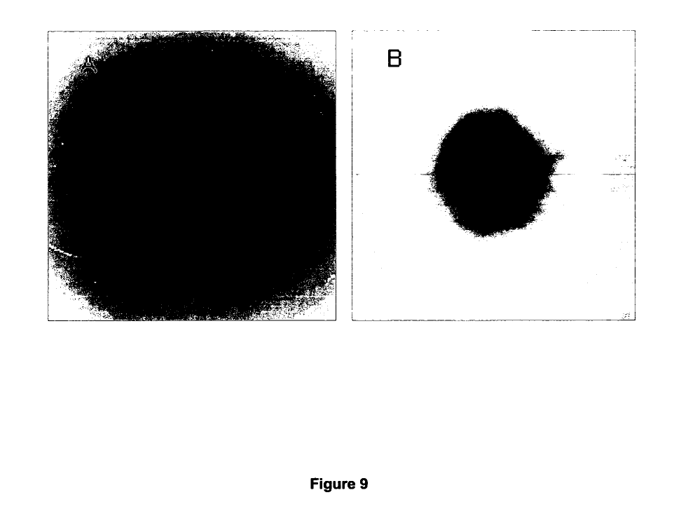

Recombinant strain G14 (transformed with pEGPL1) was re-transformed with pESCL1 using a selection with phleomycine in combination with 500 μg ml⁻¹ caffeine. This resulted in strains secreting laccase in two zones of the colony, corresponding to the zones of the single transformants. For instance strain G-S8 releases laccase both in the centre and at the peripheral part of the colony (FIG. 9), the largest part of the mycelium being devoted to secretion.

Laccase activity was determined in liquid shaken cultures in the presence of 40 g L⁻¹ of ethanol. Strain G-S8 produced more laccase than its parent G14 (Table 5).

TABLE 5
Laccase production in recombinant strains of P. cinnabarinus
BMRF 44 (laccase negative background).

| Transformant | Activity (nkat/ml) | Production (g/L) |
|---|---|---|
| S1 | 500 | 0.125 |
| L12-7 | 3600 | 0.9 |
| G14 | 4660 | 1.12 |
| G-S8 | 5310 | 1.3 |

S means that the strain has been transformed with a construct expressing Icc3-1 behind the SC3 promoter. Similarly, G and L mean that strains were transformed with a construct expressing the Icc3-1 gene from the GPD and laccase promoter, respectively. Strains are presented showing the highest activity out of 12 transformants that were selected on plate. Cultures were grown in the presence of 40 g L⁻¹ of ethanol.

1. A recombinant host cell comprising at least two DNA constructs, each DNA construct comprising a coding sequence in operative association with a promoter DNA sequence, wherein the at least two DNA constructs comprise at least two distinct promoter DNA sequences and wherein the coding sequences comprised in said DNA constructs encode related polypeptides.

2. The recombinant host cell according to claim 1, wherein the at least two distinct promoter DNA sequences possess distinct expression characteristics.

3. The recombinant host cell according to claim 1, wherein at least one of the at least two promoter DNA sequences is selected from the group consisting of:
(a) a DNA sequence comprising a nucleotide sequence selected from the set consisting of: SEQ ID NO's:1 to 4 and 13 to 55 and the promoter DNA sequences of the genes listed in Table 1,
(b) a DNA sequence capable of hybridizing with the DNA sequence of (a),
(c) a DNA sequence sharing at least 80% homology with the DNA sequence of (a),
(d) a variant of any of the DNA sequences of (a) to (c), and
(e) a subsequence of any of the DNA sequences of (a) to (d).

4. The recombinant host cell of claim 1, wherein said host cell is a fungal cell.

5. The recombinant host cell according to claim 4, wherein the host cell is an Agaricus, Aspergillus, Penicillium, Pycnoporus or Trichoderma species.

6. The recombinant host cell according to claim 5, wherein the Aspergillus is an Aspergillus niger, Aspergillus sojae, Aspergillus oryzae species.

7. The recombinant host cell according to claim 1, wherein the coding sequence encodes an enzyme.

8. The recombinant host cell according to claim 1, wherein the coding sequence encodes an enzyme involved in the production of a metabolite.

9. A method to prepare the recombinant cell of claim 1, comprising:
(a) providing at least two DNA constructs, each DNA construct comprising a coding sequence in operative association with a promoter DNA sequence, wherein the at least two DNA constructs comprise at least two distinct promoter DNA sequences and wherein the coding sequences comprised in said DNA constructs encode related polypeptides,
(b) providing a suitable host cell, and
(c) transforming said host cell with said DNA constructs.

10. The method according to claim 9, wherein step (c) is performed by at least two separate transformation events.

11. A method for the expression of a coding sequence, comprising:
(a) providing at least two DNA constructs, each DNA construct comprising a coding sequence in operative association with a promoter DNA sequence, wherein the at least two DNA constructs comprise at least two distinct promoter DNA sequences and wherein the coding sequences comprised in said DNA constructs encode related polypeptides,
(b) providing a suitable host cell,
(c) transforming said host cell with said DNA constructs,
(d) culturing said host cell under conditions conducive to expression of the coding sequence.

12. The method according to claim 11, wherein step (c) is performed by at least two separate transformation events.

13. A method for the expression of a coding sequence, comprising culturing the recombinant host according to claim 1, under conditions conducive to expression of the coding sequence.

14. A method for the production of a polypeptide, comprising:
(a) culturing a recombinant host cell comprising at least two DNA constructs, each DNA construct comprising a coding sequence in operative association with a promoter DNA sequence, wherein the at least two DNA constructs comprise at least two distinct promoter DNA sequences and wherein the coding sequences comprised in said DNA constructs encode related polypeptides, under conditions conducive to expression of the polypeptide,
(b) optionally recovering the polypeptide from the culture broth, and
(c) optionally purifying the polypeptide.

15. A method for the production of a polypeptide, comprising:
(a) culturing the recombinant host cell according to claim 1, under conditions conducive to expression of the polypeptide,
(b) optionally recovering the polypeptide from the culture broth, and
(c) optionally purifying the polypeptide.

16. A method for the production of a metabolite, comprising:
(a) culturing the recombinant host cell according to claim 8 under conditions conducive to production of the metabolite,
(b) optionally recovering the metabolite from the culture broth, and
(c) optionally purifying the metabolite.
